SEQUENCE LISTING (9 sequences)

SEQ ID NO: 1 - DNA, 1071 bp, Homo sapiens
atgaccaaatcgtacagcgagagtgggctgatgggcgagcctcagccccaaggtcctccaagctggacagacgagtgtctcagttctcaggacgaggagcacgaggcagacaagaaggaggacgacctcgaagccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatcctgcgctcaggcaaaagcccagacctggtctccttcgttcagacgctttgcaagggcttatcccaacccaccaccaacctggttgcgggctgcctgcaactcaatcctcggacttttctgcctgagcagaaccaggacatgcccccccacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctcaatgccatatttcatgattag

SEQ ID NO: 2 - protein, 356 aa, Homo sapiens
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIMSFDSHSHHERVMSAQLNAIFHD

SEQ ID NO: 3 - DNA, 1074 bp, Mus musculus
atgaccaaatcatacagcgagagcgggctgatgggcgagcctcagccccaaggtcccccaagctggacagatgagtgtctcagttctcaggacgaggaacacgaggcagacaagaaagaggacgagcttgaagccatgaatgcagaggaggactctctgagaaacgggggagaggaggaggaggaagatgaggatctagaggaagaggaggaagaagaagaggaggaggaggatcaaaagcccaagagacggggtcccaaaaagaaaaagatgaccaaggcgcgcctagaacgttttaaattaaggcgcatgaaggccaacgcccgcgagcggaaccgcatgcacgggctgaacgcggcgctggacaacctgcgcaaggtggtaccttgctactccaagacccagaaactgtctaaaatagagacactgcgcttggccaagaactacatctgggctctgtcagagatcctgcgctcaggcaaaagccctgatctggtctccttcgtacagacgctctgcaaaggtttgtcccagcccactaccaatttggtcgccggctgcctgcagctcaaccctcggactttcttgcctgagcagaacccggacatgcccccgcatctgccaaccgccagcgcttccttcccggtgcatccctactcctaccagtcccctggactgcccagcccgccctacggcaccatggacagctcccacgtcttccacgtcaagccgccgccacacgcctacagcgcagctctggagcccttctttgaaagccccctaactgactgcaccagcccttcctttgacggacccctcagcccgccgctcagcatcaatggcaacttctctttcaaacacgaaccatccgccgagtttgaaaaaaattatgcctttaccatgcactaccctgcagcgacgctggcagggccccaaagccacggatcaatcttctcttccggtgccgctgcccctcgctgcgagatccccatagacaacattatgtctttcgatagccattcgcatcatgagcgagtcatgagtgcccagcttaatgccatctttcacgattag

SEQ ID NO: 4 - protein, 357 aa, Mus musculus
MTKSYSESGLMGEPQPQGPPSWTDECLSSQDEEHEADKKEDELEAMNAEEDSLRNGGEEEEEDEDLEEEEEEEEEEEDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNPDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGPQSHGSIFSSGAAAPRCEIPIDNIMSFDSHSHHERVMSAQLNAIFHD

SEQ ID NO: 5 - DNA, 1053 bp, Mus musculus
gcagtgtggagacacacccactttccccaagggctcctgctcccccaagtgatccccttatcctccgtgctaagatgacaccgaggttgcagtccttacctttgaaagcagccacaagggcgtgggggtgcacacctttaatcccagcactcgggaggcagaggcaggcagatttctgagttcgagaccagcctggtctacaaagtgaattccaggacagccagggctatacagagaaaccctgtcttgaaaaaaaaagagaaagaaaaaagaaaaaaaaaaatgaaagcagccacatctaaggactacgtggcacaggagagggtgagtccctgagagttcagctgctgccctgtctgttcctgtaaatggcagtggggtcatgggaaagtgaaggggctcaaggtattggacacttccaggataatcttttggacgcctcaccctgtgccaggaccaaggctgagcttggcaggctcagaacagggtgtcctgttcttccctgtctaaaacattcactctcagcttgctcacccttccccagacaaggaagctgcacagggtctggtgttcagatggctttggcttacagcaggtgtgggtgtggggtaggaggcagggggtaggggtgggggaagcctgtactatactcactatcctgtttctgaccctctaggactcctacagggttatgggagtggacaggcagtccagatctgagctgctgacccacaagcagtgccctgtgcctgccagaatccaaagccctgggaatgtccctctggtccccctctgtcccctgcagcccttcctgttgctcaaccttgcacagttccgacctgggggagagagggacagaaatcttgccaagtatttcaacagaatgtactggcaattacttcatggcttcctggacttggtaaaggatggactaccccgcccaacaggggggctggcagccaggtaggcccataaaaagcccgctggggagtcctcctcactctctgctcttcctcctccagcacacatcagacctagtagctgtggaaacca

SEQ ID NO: 6 - DNA, 1672 bp, Homo sapiens
gtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagtagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgcgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgattcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcat

SEQ ID NO: 7 - DNA, 1610 bp, Mus musculus
aactgagagtggaggggcacagaagagcccaagaggctccttaggttgtgtggagggtacaatatgtttgggctgagcaacccagagccagactttgtctggctggtaagagacagaggtgcctgctatcacaatccaagggtctgcttgaggcagagccagtgcaaaggatgtggttagagccagcctggtgtactgaagaggggcgaagagcttgagtaaggagtctcagcggtggtttgagaggcagggtggttaatggagtagctgcaggggagaatccttgggagggagcctgcaggacagagctttggtcaggaagtgatgggcatgtcactggaccctgtattgtctctgacttttctcaagtaggacaatgactctgcccagggagggggtctgtgacaaggtggaagggccagaggagaacttctgagaagaaaaccagaggccgtgaagaggtgggaagggcatgggattcagaacctcaggcccaccaggacacaaccccaggtccacagcagatgggtgaccttgcatgtctcagtcaccagcattgtgctccttgcttatcacgcttgggtgaaggaaatgacccaaatagcataaagcctgaaggccgggactaggccagctagggcttgcccttcccttcccagctgcactttccataggtcccaccttcagcagattagacccgcctcctgcttcctgcctccttgcctcctcactcatgggtctatgcccacctccagtctcgggactgaggctcactgaagtcccatcgaggtctggtctggtgaatcagcggctggctctgggccctgggcgaccagttaggttccgggcatgctaggcaatgaactctacccggaattgggggtgcggggaggcggggaggtctccaacccagccttttgaggacgtgcctgtcgctgcacggtgctttttatagacgatggtggcccattttgcagaagggaaagccggagccctctggggagcaaggtccccgcaaatggacggatgacctgagcttggttctgccagtccacttcccaaatccctcaccccattctagggactagggaaagatctcctgattggtcatatctgggggcctggccggagggcctcctatgattggagagatctaggctgggcgggccctagagcccgcctcttctctgcctggaggaggagcactgaccctaaccctctctgcacaagacccgagcttgtgcgcccttctgggagcttgctgcccctgtgctgactgctgacagctgactgacgctcgcagctagcaggtacttctgggttgctagcccagagccctgggccggtgaccctgttttccctacttcccgtctttgaccttgggtaagtttctttttcttttgtttttgagagaggcacccagatcctctccactacaggcagccgctgaaccttggatcctcagctcctgccctgggaactacagttcctgccctttttttcccaccttgagggaggttttccctgagtagcttcgactatcctggaacaagctttgtagaccagcctgggtctccggagagttgggattaaaggcgtgcaccaccacc

SEQ ID NO: 8 - DNA, 1387 bp, Homo sapiens
ctctggtttcaagaccaatactcataacccccacatggaccaggcaccatcacacctgagcactgcacttagggtcaaagacctggccccacatctcagcagctatgtagactagctccagtcccttaatctctctcagcctcagtttcttcatctgcaaaacaggtctcagtttcgttgcaaagtatgaagtgctgggctgttactggtcaaagggaagagctgggaagagggtgcaaggtggggttgggctggagatgggctggagcagatagatggagggacctgaatggaggaagtaaaccaaggcccggtaacattgggactggacagagaacacgcagatcctctaggcaccggaagctaagtaacattgccctttctcctcctgtttgggactaggctgatgttgctgcctggaagggagccagcagaagggccccagcctgaagctgttaggtagaagccaaatccagggccagatttccaggaggcagcctcgggaagttgaaacacccggattcaggggtcaggaggcctgggcttctggcaccaaacggccagggacctactttccacctggagtcttgtaagagccactttcagcttgagctgcactttcgtcctccatgaaatgggggaggggatgctcctcacccaccttgcaaggttattttgaggcaaatgtcatggcgggactgagaattcttctgccctgcgaggaaatccagacatctctcccttacagacagggagactgaggtgaggcccttccaggcagagaaggtcactgttgcagccatgggcagtgccccacaggacctcgggtggtgcctctggagtctggagaagttcctaggggacctccgaggcaaagcagcccaaaagccgcctgtgagggtggctggtgtctgtccttcctcctaaggctggagtgtgcctgtggaggggtctcctgaactcccgcaaaggcagaaaggagggaagtaggggctgggacagttcatgcctcctccctgagggggtctcccgggctcggctcttggggccagagttcagggtgtctgggcctctctatgactttgttctaagtctttagggtggggctggggtctggcccagctgcaagggccccctcacccctgccccagagaggaacagccccgcacgggccctttaagaaggttgagggtgggggcaggtgggggagtccaagcctgaaacccgagcgggcgcgcgggtctgcgcctgccccgcccccggagttaagtgcgcggacacccggagccggcccgcgcccaggagcagagccgcgctcgctccactcagctcccagctcccaggactccgctggctcctcgcaagtcctgccgcccagcccgccggg

SEQ ID NO: 9 - DNA, 9232 bp, Artificial Sequence
  Construct including CAG promoter and encoding NeuroD1 and green fluoresecent protein separated by an IRES.
gatccggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcatgtacggtgggaggtctatataagcagagctcaataaaagagcccacaacccctcactcggggcgccagtcctccgattgactgagtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcgatgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagagcctctgctaaccatgttcatgccttcttctttttcctacagctcctgggcaacgtgctggttgttgtgctgtctcatcattttggcaaagaattcgctagcggatccggccgcctcggccaccggtcgccaccatcgccaccatgaccaaatcatacagcgagagcgggctgatgggcgagcctcagccccaaggtcccccaagctggacagatgagtgtctcagttctcaggacgaggaacacgaggcagacaagaaagaggacgagcttgaagccatgaatgcagaggaggactctctgagaaacgggggagaggaggaggaggaagatgaggatctagaggaagaggaggaagaagaagaggaggaggaggatcaaaagcccaagagacggggtcccaaaaagaaaaagatgaccaaggcgcgcctagaacgttttaaattaaggcgcatgaaggccaacgcccgcgagcggaaccgcatgcacgggctgaacgcggcgctggacaacctgcgcaaggtggtaccttgctactccaagacccagaaactgtctaaaatagagacactgcgcttggccaagaactacatctgggctctgtcagagatcctgcgctcaggcaaaagccctgatctggtctccttcgtacagacgctctgcaaaggtttgtcccagcccactaccaatttggtcgccggctgcctgcagctcaaccctcggactttcttgcctgagcagaacccggacatgcccccgcatctgccaaccgccagcgcttccttcccggtgcatccctactcctaccagtcccctggactgcccagcccgccctacggcaccatggacagctcccacgtcttccacgtcaagccgccgccacacgcctacagcgcagctctggagcccttctttgaaagccccctaactgactgcaccagcccttcctttgacggacccctcagcccgccgctcagcatcaatggcaacttctctttcaaacacgaaccatccgccgagtttgaaaaaaattatgcctttaccatgcactaccctgcagcgacgctggcagggccccaaagccacggatcaatcttctcttccggtgccgctgcccctcgctgcgagatccccatagacaacattatgtctttcgatagccattcgcatcatgagcgagtcatgagtgcccagcttaatgccatctttcacgattaggtttaaacgcggccgcgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcgagcttgttaacatcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttccgacttgtggtctcgctgccttgggagggtctcctctgagtgattgactacccgtcagcgggggtcttcacatgcagcatgtatcaaaattaatttggttttttttcttaagtatttacattaaatggccatagttgcattaatgaatcggccaacgcgcggggagaggcggtttgcgtattggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagtttgcggccggccgcaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaat

(19) United States
(12) Patent Application Publication
(10) Pub. No.: US 2022/0160825 A1
(43) Pub. Date: May 26, 2022

(54) BRAIN REPAIR AFTER TRAUMATIC BRAIN INJURY THROUGH NEUROD1-MEDIATED ASTROCYTE-TO-NEURON CONVERSION

(71) Applicant: The Penn State Research Foundation, University Park, PA (US)

(72) Inventors: Gong Chen, State College, PA (US); Zhuo-Fan Lei, Lutherville Timonium, MD (US)

(73) Assignee: The Penn State Research Foundation, University Park, PA (US)

(21) Appl. No.: 17/105,022

(22) Filed: Nov. 25, 2020

(51) Int. Cl. A61K 38/17; A61P 25/00; C12N 15/86

(52) U.S. Cl. CPC A61K 38/1709; A61P 25/00; C12N 2750/14171; C12N 2750/14143; C12N 15/86

(57) ABSTRACT

Methods of treating traumatic brain injury (TBI) are provided according to aspects of the present disclosure including: converting reactive astrocytes to functional neurons by providing exogenous neurogenic differentiation 1 (NeuroD1, also called ND1 herein) to at least one reactive astrocyte in a damaged region of a subject's brain, such as the brain of a human subject with a TBI. According to aspects, presence of non-functional neurons and reactive astrocytes in the damaged region of the subject's brain are not primarily due to bleeding and/or ischemia in the damaged region. According to aspects of the present disclosure, the traumatic brain injury causes a period of astrogliosis in the damaged region of the subject's brain, and the exogenous NeuroD1 is provided to reactive astrocytes in the damaged region of the subject's brain during the period of astrogliosis or within four weeks after the period of astrogliosis.

### CROSS-REFERENCE TO RELATED APPLICATION

This application claims priority to U.S. provisional application No. 62/939,978, filed Nov. 25, 2019, which is incorporated by reference herein in its entirety.

### SEQUENCE LISTING

A sequence listing contained in the file name 36PST97602PA_ST25.txt which is 29,536 bytes in size (measured in MS-Windows®) and created on Nov. 25, 2020, is filed electronically herewith and incorporated by reference in its entirety.

### BACKGROUND OF THE INVENTION

Traumatic brain injury (TBI) is one of the leading causes of death and disability all over the world. The CDC has reported that about 1.7 million people needed medical care for TBI each year in the US, at a cost of more than 77 billion dollars yearly. Worldwide, 50 million people are affected by TBI at a cost of 400 billion dollars annually (Maas et al., Lancet Neurol., 16(12):987-1048, 2017).

TBI causes acute damage to the brain tissue, and also results in secondary injuries to the nervous system, leading to the consequences of chronic physical and/or mental deficits. TBI results in blood brain barrier breakdown, microgliosis, astrogliosis, and neuronal degeneration. The adult mammalian brain lacks the ability to regenerate neurons after injury and there is a lack of treatments capable of promoting neuronal regeneration following TBI. There is a continuing need for treatments promoting repair of the damaged brain after TBI.

### SUMMARY OF THE INVENTION

Methods of treating traumatic brain injury (TBI) are provided according to aspects of the present disclosure including: converting reactive astrocytes to functional neurons by providing exogenous neurogenic differentiation 1 (NeuroD1, also called ND1 herein) to at least one reactive astrocyte in a damaged region of a subject's brain. According to aspects, the TBI is a closed head injury. According to aspects of the present disclosure, the damaged region of the brain includes non-functional neurons and reactive astrocytes due to the TBI. According to aspects of the present disclosure, the non-functional neurons are selected from the group consisting of dead neurons, dying neurons, and a combination thereof. According to aspects of the present disclosure, non-functional neurons present in the damaged region of the brain are detected by a functional MRI (fMRI). According to aspects of the present disclosure, the subject is human.

Methods of treating TBI are provided according to aspects of the present disclosure including: converting reactive astrocytes to functional neurons by providing exogenous NeuroD1 to at least one reactive astrocyte in a damaged region of a subject's brain wherein the damaged region of the brain includes non-functional neurons and reactive astrocytes due to the TBI. According to aspects of the present disclosure the presence of non-functional neurons and reactive astrocytes in the damaged region are not primarily due to bleeding in the damaged region. According to aspects of the present disclosure the presence of non-functional neurons and reactive astrocytes are not primarily due to ischemia in the damaged region. According to aspects, the TBI is a closed head injury. According to aspects of the present disclosure, the non-functional neurons are dead neurons. According to aspects of the present disclosure the non-functional neurons are dying neurons. According to aspects of the present disclosure, non-functional neurons present in the damaged region of the brain are detected by a functional MM (fMRI). According to aspects of the present disclosure, the subject is human.

According to aspects of the present disclosure, providing the exogenous NeuroD1 includes providing exogenous NeuroD1 to the at least one reactive astrocyte at a first treatment time in the range of about two days to about ten days after the traumatic brain injury.

According to aspects of the present disclosure, the traumatic brain injury causes a period of astrogliosis in the damaged region, and wherein providing the exogenous NeuroD1 includes providing exogenous NeuroD1 to the at least one reactive astrocyte at a first treatment time during the period of astrogliosis or within four weeks after the period of astrogliosis.

According to aspects of the present disclosure, providing the exogenous NeuroD1 includes providing exogenous NeuroD1 to the at least one reactive astrocyte at a second treatment time after the first treatment time and during the period of astrogliosis or within four weeks after the period of astrogliosis.

According to aspects of the present disclosure, providing the exogenous NeuroD1 includes providing exogenous NeuroD1 to the at least one reactive astrocyte at a third treatment time after the second treatment time and during the period of astrogliosis or within four weeks after the period of astrogliosis.

According to aspects of the present disclosure, providing the exogenous NeuroD1 includes administering a recombinant expression vector to the subject, wherein the recombinant expression vector includes a nucleic acid sequence encoding NeuroD1.

According to aspects of the present disclosure, providing the exogenous NeuroD1 includes administering a recombinant expression vector to the subject, wherein the recombinant expression vector is a viral expression vector including a nucleic acid sequence encoding NeuroD1.

According to aspects of the present disclosure, providing the exogenous NeuroD1 includes administering a recombinant expression vector to the subject, wherein the recombinant expression vector is a recombinant adeno-associated virus expression vector, and wherein the recombinant adeno-associated virus vector includes a nucleic acid sequence encoding NeuroD1.

According to aspects of the present disclosure, the nucleic acid sequence encoding NeuroD1 is operably linked to a promoter.

According to aspects of the present disclosure, the promoter is a glial-cell specific promoter.

According to aspects of the present disclosure, the glial-cell specific promoter is a glial fibrillary acidic protein (GFAP) promoter.

According to aspects of the present disclosure, the GFAP promoter is a human GFAP (hGFP) promoter.

According to aspects of the present disclosure, no exogenous transcription factor other than NeuroD1 is provided to the at least one reactive astrocyte.

According to aspects of the present disclosure, the NeuroD1 includes an amino acid sequence selected from the group consisting of: SEQ ID NO: 2, SEQ ID NO: 4, a functional fragment of SEQ ID NO: 2, a functional fragment of SEQ ID NO: 4, an amino acid sequence having at least 85% identity to SEQ ID NO: 2, and an amino acid sequence having at least 85% identity to SEQ ID NO: 4.

According to aspects of the present disclosure, the NeuroD1 is encoded by a nucleic acid sequence including SEQ ID NO: 1, a nucleic acid sequence having at least 85% identity to SEQ ID NO: 1, a nucleic acid sequence including SEQ ID NO: 3, or a nucleic acid sequence having at least 85% identity to SEQ ID NO: 3.

According to aspects of the present disclosure, providing the exogenous NeuroD1 includes injection into the damaged region of the brain.

According to aspects of the present disclosure, the nucleic acid sequence encoding NeuroD1 is present in a virus particle.

According to aspects of the present disclosure, providing the exogenous NeuroD1 includes administering about 10⁷ to about 10¹⁴ virus particles to the damaged brain region of the subject.

Uses of a composition including NeuroD1 are provided in the manufacture of a medicament for converting reactive astrocytes to functional neurons in a damaged region of a subject's brain, wherein the damaged region of the brain includes non-functional neurons and reactive astrocytes, due to a TBI. According to aspects of the present disclosure, the non-functional neurons are dead neurons. According to aspects of the present disclosure, the non-functional neurons are dying neurons. According to aspects of the present disclosure, the traumatic brain injury is a closed head injury. According to aspects of the present disclosure, the NeuroD1 is encoded by a nucleic acid sequence includes a nucleic acid sequence having at least 85% identity to SEQ ID NO: 1. According to aspects of the present disclosure, the nucleic acid encoding NeuroD1 includes a nucleic acid sequence having at least 85% identity to SEQ ID NO: 3. According to aspects of the present disclosure, the NeuroD1 includes an amino acid sequence selected from the group consisting of: SEQ ID NO: 2, SEQ ID NO: 4, a functional fragment of SEQ ID NO: 2, a functional fragment of SEQ ID NO: 4, an amino acid sequence having at least 85% identity to SEQ ID NO: 2, and an amino acid sequence having at least 85% identity to SEQ ID NO: 4.

According to aspects of the present disclosure, the NeuroD1 is encoded by a nucleic acid sequence included in a recombinant expression vector. According to aspects of the present disclosure, the nucleic acid sequence encoding NeuroD1 is operably linked to a promoter. According to aspects of the present disclosure, the promoter is a glial-cell specific promoter. According to aspects of the present disclosure, the glial-cell specific promoter is a GFAP promoter. According to aspects of the present disclosure, the GFAP promoter is an hGFP promoter. According to aspects of the present disclosure, the NeuroD1 is encoded by a nucleic acid sequence included a viral expression vector. According to aspects of the present disclosure, the NeuroD1 is encoded by a nucleic acid sequence included a recombinant adeno-associated virus expression vector.

### BRIEF DESCRIPTION OF THE DRAWINGS

FIG. 1A shows aspects of establishment of a focal closed head injury model for study of treatment of traumatic brain injury; the model includes used of an electric-magnet controlled device to induce a focal closed head injury to the motor cortex shown diagrammatically on a mouse head;

FIG. 1B is a schematic illustration of the timeline for injury induction and pathology investigation;

FIG. 1C is a set of images showing results of immunostaining for a neuronal marker (NeuN) and an astrocytic marker (GFAP) which reflected the cell density of surviving neurons and reactive astrocytes in mouse brain from sham-traumatic brain injury mice (Sham-TBI group) or in mouse brain from traumatic brain injury mice (TBI group) at the indicated time points following traumatic brain injury;

FIG. 1D is a graph showing NeuN density significantly decreased in the injury core;

FIG. 1E is a graph showing NeuN density significantly decreased in the peri-injury area;

FIG. 1F is a graph showing that reactive astrocyte density significantly increased in the total injury area;

FIG. 1G is a set of images showing results of co-immunostaining for microglia marker (Iba1), astrocytic marker (GFAP), and cell proliferation marker (Ki67) in mouse brain from sham-traumatic brain injury mice (Sham-TBI group) or in mouse brain from traumatic brain injury mice (TBI group) at the indicated time points following traumatic brain injury, showing the neuroinflammation process at these early time points after TBI;

FIG. 1H is a graph showing that proliferation rate of microglia cells reached a peak around 1 day after TBI and proliferation rate of astrocytes reached a peak around 4 days after TBI;

FIG. 2A illustrates the definition of injury core and peri-injury area of mouse motor cortex in a CHI model;

FIG. 2B is a set of images showing results of immunostaining of damaged brain tissue at early time points, 6 hours and 4 days, after CHI. The immunostaining results showed that a cell apoptosis marker, TUNEL, colocalized with a neuronal marker, NeuN, which suggested that there would be death and loss of many neurons, especially in the superficial layer of the damaged motor cortex;

FIG. 2C is a set of images showing results of immunostaining of damaged brain tissue for myelin basic protein (MBP) and neurofilament protein (NF200) at 7 days after CHI. The immunostaining results demonstrated that neuronal processes were damaged at the injury site after CHI;

FIG. 3A shows diagrammatic Illustrations of the closed head injury in mouse motor cortex and administration of ND1 at or near the impact site after the CHI.

FIG. 3B diagrammatically shows an experimental scheme of CHI induction, NeuroD1-encoding virus injection and immunofluorescence experiments described in detail in Examples herein;

FIG. 3C is a set of representative images showing the injured cortex 7 days after injection of AAV-GFAP::GFP virus (control group, left panel) or injection of AAV-GFAP::ND1-GFP virus (ND1 group, right panel);

FIG. 3D is a set of images showing GFP fluorescence and immunofluorescence of the indicated marker; as shown, under GFAP promotor control, GFP was mainly expressed in GFAP+ astrocytes, whereas very low GFP expression was found in other cortical cells of different subtypes at 7 days after AAV-GFAP::GFP virus injection in control group;

FIG. 3E is a set of “zoomed-in” images from FIG. 2C illustrating that NeuroD1 was highly expressed in GFP+ astrocytes in the ND1 group 7 days after AAV-GFAP::ND1-GFP virus injection (lower panels) compared to control group (upper panels);

FIG. 3F is a set of images showing results of co-staining for GFAP, NeuN and ND1 which showed the astrocyte-to-neuron conversion process at different time points after AAV-GFAP::ND1-GFP virus injection;

FIG. 3G is a graph showing quantification of the percentage of different types among the total GFP-expressing cortical cells were shown in FIG. 3D;

FIG. 3H is a graph showing quantification of the percentage of cells expressing a neuronal marker, NeuN, with GFP at different time points after AAV-GFAP::ND1-GFP virus injection;

FIG. 4A is a set of images from damaged brain at 4 days after CHI, illustrating that some GFP+ cells showed both GFAP and NeuN signal at the same time, which indicated that they were in the transitional stage from reactive astrocyte to neuron;

FIG. 4B is a set of images showing that, among the converted neurons, the variation trend of immature neuron marker (Tuj1) and mature neuron marker (MAP2) implied that converted neurons became mature gradually;

FIG. 4C is a set of images showing GFP fluorescence, NeuN immunofluorescence, and GFAP immunofluorescence and showing that “astrocyte to neuron” (AtN) conversion by NeuroD1 was confirmed using (retrovirus) CAG::ND1-GFP or (retrovirus) CAG::GFP expression constructs

FIG. 4D is a graph showing that “astrocyte to neuron” (AtN) conversion by NeuroD1 was confirmed using (retrovirus) CAG::ND1-GFP or (retrovirus) CAG::GFP expression constructs and that retrovirus carrying ND1 converted about half of GFP-expressing cells to NeuN+, while there was no conversion of astrocytes to neurons in the control group;

FIG. 5A is a set of images showing that most converted neurons showed FoxG1 signal and many converted neurons showed Tbr1 signal;

FIG. 5B is an image showing that, after ND1 treatment, immunostaining with the superficial cortical marker (Cux1) and deep layer marker (Ctip2) suggested that cortical layers were still well organized.

FIG. 5C is a set of images showing that some converted neurons were Cux1+ or Ctip2+ in superficial layer or deep layer in mouse cortex;

FIG. 5D is a graph showing results of quantification of the percentage of converted neurons expressing cortical markers FoxG1, and/or Tbr1, or layer markers Cux1, and/or Ctip2, with GFP and NeuN at 28 days after GFAP::ND1-GFP virus injection;

FIG. 6A is a set of images showing that, at 28 days after ND1 treatment, some converted neurons had both GABA and GAD67 signal inside cell soma, which indicated that they were GABAergic neurons;

FIG. 6B is a set of images showing that some converted neurons could be positive for markers of different subtypes of GABAergic neurons in mouse cortex, like Pavabulmin, Calretinin, Neuropeptide Y, Somatostatin;

FIG. 6C is a graph showing quantification of the percentage of cells expressing neuron subtype markers 28 days after AAV-GFAP::ND1-GFP virus injection;

FIG. 7A is a set of images showing morphology of converted neurons at a, b, and c, along with GFP fluorescence and NeuN immunofluorescence;

FIG. 7B is a set of three traces of action potential firing patterns obtained by whole cell patch recording representative of three different action potential firing patterns, I, II, and III;

FIG. 7C is a pie chart graph showing results of quantitation of converted neurons having either action potential firing pattern I, II, or III;

FIG. 7D is a trace showing that converted neurons fired sEPSCs of which the frequency and amplitude was higher than those from wild type control;

FIG. 7E is a trace showing that converted neurons fired sIPSCs of which the frequency and amplitude was higher than those from wild type control;

FIG. 7F is a set of graphs showing that converted neurons fired sEPSCs of which the frequency and amplitude was higher than those from wild type control;

FIG. 7G is a set of graphs showing that converted neurons fired sIPSCs of which the frequency and amplitude was higher than those from wild type control;

FIG. 8A is a graph demonstrating that the frequency of sEPSCs showed a trend of increase at early time points, and then decreased at later time points to the control level;

FIG. 8B is a graph demonstrating that the amplitude of sEPSCs increased significantly after the first week post-NeuroD1 administration, then went down to the control level two months later;

FIG. 8C is a diagram showing an experimental scheme for showing neural innervation on converted neurons at an early time point (day 7) post-NeuroD1 administration;

FIG. 8D is a set of images illustrating colocalization of a synaptic marker (VGAT) with GFP and NeuN in the cell soma of converted neurons at 7 days after NeuroD1 virus injection and CTB-647 injection on the contralateral side; CTB signal from contralateral side was also observed on the cell soma;

FIG. 8E is a set of images illustrating colocalization of a synaptic vesicle marker (SV2) with GFP and NeuN in the cell soma of converted neurons at 7 days after NeuroD1 virus injection and CTB-647 injection on the contralateral side; CTB signal from contralateral side was also observed on the cell soma;

FIG. 9A is a set of images showing that a glutamatergic synaptic marker (vGlut1), or a GABAergic synaptic marker (vGAT), colocalize with GPF on the cell soma of ND1 converted neurons;

FIG. 9B is a set of images showing that a synaptic terminal marker (synaptophysin, SP1), or a synaptic vesicle marker (SV2), colocalize with GPF around the cell boundary of ND1 converted neurons;

FIG. 9C is a set of images showing that ND1 converted neurons demonstrated comparable cFos expression with endogenous neurons in mouse motor cortex;

FIGS. 9D-9F show that thalamus neurons were labeled by (AAV)Synapsin::Cre+CAG::Flex-mCherry in the NeuroD1 group for anterograde tracing;

FIG. 9D is an image illustrating that, for anterograde tracing in mice to which the ND1-GFP expressing virus was administered, viruses AAV-synapsin::Cre+AAV-CAG::FlexmCherry (which express a red fluorescent protein, mCherry) were further injected into mouse thalamus, thereby labeling neurons to visualize their axon projections onto ND1 converted neurons expressing GFP;

FIG. 9E is a set of images showing an ND1 converted neuron which had GFP-containing synaptic boutons on the soma illustrating local innervation from other converted neurons;

FIG. 9F is a set of images showing an ND1 converted neuron which had mCherry-containing synaptic boutons on the soma illustrating innervation from remote thalamus neurons;

FIG. 9G is a set of images showing that CTB-467 was injected for retrograde tracing in the contralateral side to the NeuroD1-expressing virus injection site and CTB signal was found in some converted neurons; and

FIG. 9H is a set of graphs showing that the average CTB signal inside converted neurons increased over time after the NeuroD1-expressing virus was injected as the conversion process proceeded; CTB was injected 7 days before the brain samples were acquired for all the indicated time points.

### DETAILED DESCRIPTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Herdewijn, P. (Ed.), Oligonucleotide Synthesis: Methods and Applications, Methods in Molecular Biology, Humana Press, 2004; Remington: The Science and Practice of Pharmacy, Lippincott Williams & Wilkins, 21st Ed., 2005; L. V. Allen, Jr. et al., Ansel's Pharmaceutical Dosage Forms and Drug Delivery Systems, 8th Ed., Philadelphia, Pa.: Lippincott, Williams & Wilkins, 2004; and L. Brunton et al., Goodman & Gilman's The Pharmacological Basis of Therapeutics, McGraw-Hill Professional, 12th Ed., 2011.

The singular terms “a,” “an,” and “the” are not intended to be limiting and include plural referents unless explicitly stated otherwise or the context clearly indicates otherwise.

When a grouping of alternatives is presented, any and all combinations of the members that make up that grouping of alternatives is specifically envisioned. For example, if an item is selected from a group consisting of A, B, C, and D, each alternative individually (e.g., A alone, B alone, etc.), as well as combinations such as A, B, and D; A and C; B and C; etc., is envisioned. The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

When a range of numbers is provided herein, the range is understood to be inclusive of the edges of the range as well as any number between the defined edges of the range. For example, “between 1 and 10” includes any number between 1 and 10, as well as the number 1 and the number 10.

When the term “about” is used in reference to a number, it is understood to mean plus or minus 10%. For example, “about 100” would include from 90 to 110.

Compositions and methods for treating traumatic brain injury (TBI) in a subject are provided according to aspects of the present disclosure.

Methods effective to reverse the neuronal loss resulting from TBI are provided according to aspects of the present disclosure. Unexpectedly, expression of exogenous neurogenic differentiation 1 (NeuroD1) in glial cells, particularly astrocytes and/or reactive astrocytes, treats TBI in a subject in need thereof. Thus, provided by the present disclosure are methods of treatment of TBI in a subject, including administration of a therapeutically effective amount of NeuroD1 to the subject.

Methods of treating traumatic brain injury (TBI) are provided according to aspects of the present disclosure which include converting reactive astrocytes to functional neurons by providing exogenous NeuroD1 to at least one reactive astrocyte in a damaged region of a subject's brain.

The term “NeuroD1” refers to a bHLH proneural transcription factor, neurogenic differentiation 1, involved in embryonic brain development and in adult neurogenesis, see Cho, J. H. et al., Mol, Neurobiol., 30:35-47, 2004; Kuwabara, T. et al., Nature Neurosci., 12: 1097-1105, 2009; and Gao, Z. et al., Nature Neurosci., 12:1090-1092, 2009. NeuroD1 is expressed late in development, mainly in the nervous system and is involved in neuronal differentiation, maturation and survival.

The term “exogenous” used herein to refer to NeuroD1 refers to NeuroD1 present in a glial cell, particularly an astrocyte and/or reactive astrocyte, to be converted to a functional neuron by operation of a method of the present disclosure and is not naturally present in the glial cell.

The term “functional” with respect to a neuron, as used herein, refers to a neuron exhibiting and/or a maintaining a capability to perform an action and/or task for which the neuron is specially fitted or exists to perform.

The terms “treat,” “treatment,” “treating” and “NeuroD1 treatment” or grammatical equivalents as used herein refer to alleviating, inhibiting or ameliorating a TBI, symptoms or signs of a TBI, and preventing symptoms or signs of TBI, and include, but are not limited to therapeutic and/or prophylactic treatments.

The term “therapeutically effective amount” as used herein is intended to mean an amount of an inventive composition which is effective to alleviate, ameliorate or prevent a symptom or sign of a TBI to be treated. According to aspects of the present disclosure, a therapeutically effective amount is an amount which has a beneficial effect in a subject having signs and/or symptoms of TBI. According to aspects of the present disclosure, administration of a therapeutically effective amount of NeuroD1 to a subject affected by a TBI provides the generation of new functional neurons by conversion of reactive astrocytes to functional neurons; reduction of the number of reactive astrocytes; the generation of new non-reactive astrocytes; and integration of the new functional neurons into the neuronal network both in the injured region and in non-injured regions of the brain of the subject.

The term “traumatic brain injury,” abbreviated “TBI” herein, refers to a sudden injury to the brain which can be either a closed head injury (CHI) due to an impact to the head, or a penetrating head injury. A non-limiting example of a penetrating head injury is an object piercing the skull and entering the brain. According to aspects of the present disclosure, a TBI is a CHI. According to aspects of the present disclosure, a TBI is a penetrating head injury. TBI can result from direct impact to the head from any of various sources such as, but not limited to, a fall, car accidents, sports accidents, being struck with an object, or an indirect impact such as shock waves from an explosion. A non-limiting example of an explosion is a battlefield explosion. According to aspects of the present disclosure a TBI results from a fall. According to aspects of the present disclosure a TBI results from a car accident. According to aspects of the present disclosure a TBI results from a sports accident. According to aspects of the present disclosure a TBI results from being struck by an object. According to aspects of the present disclosure a TBI results from an indirect impact such as shock waves from an explosion.

According to aspect of the present disclosure, a non-limiting example of a TBI is a brain injury resulting from an impact to the head of a subject which is alleviated, ameliorated or prevented by additional functional neurons.

The term “closed head injury,” abbreviated “CHI” herein, refers to a TBI due to a non-penetrating injury to the head of a subject or an injury to the head that did not fracture and/or compromise the integrity of the skull.

According to aspects of the present disclosure, the TBI is “focal” such that primary damage to the brain is localized to an area of the brain adjacent to the impact site. Secondary damage to the brain may be present in other regions of the brain resulting from the primary damage.

The term “primary damage” refers to presence of non-functional neurons, such as dead and/or dying neurons, and reactive astrocytes, in the area adjacent the impact site, wherein presence of the non-functional neurons, such as dead and/or dying neurons, and reactive astrocytes in the damaged region are not primarily due to bleeding and/or ischemia in the damaged region. According to aspects of the present disclosure, dead or dying neurons are measured by apoptotic assays and functional assay. Non-limiting examples of apoptotic assays include electron microscopy, TUNEL assay, flow cytometry, the DNA ladder assay, detection of cytochrome c, detection of annexin V, and caspase activity assays. Non-limiting examples of functional assays include functional magnetic resonance imaging (fMRI). According to aspects of the present disclosure, TBI may result from two or more impacts and that each of the two or more impacts is associated with an impact site such that primary damage to the brain is associated to each of the two or more impact sites.

The therapeutically effective amount of NeuroD1 in the glial cells treats at least one sign and/or symptom of TBI in the subject, whereby the TBI is treated.

Signs and symptoms of TBI are well-known in the art along with methods of detection and assessment of such signs and symptoms. Signs and symptoms of TBI in a subject include loss of consciousness, confusion, disorientation, headache, fatigue, speech problems, sleep problems, dizziness, balance problems, sensory problems, sensitivity to light, loss of sight or changes in vision, loss or alterations in the sense of smell, loss or alterations in the sense of taste, tinnitus, loss or alterations in the sense of hearing, memory problems, concentration problems, depression, anxiety, agitation, mood swings, seizures, loss or diminishment of coordination, motor issues, cognitive issues including difficulty learning, negative changes in reasoning ability, negative changes in judgement, and negative changes in attention or concentration.

Signs and/or symptoms of TBI in a subject include presence of non-functional neurons, such as dead and/or dying neurons, in the region of the brain damaged due to the TBI. The number of dead and/or dying neurons in the region of the brain damaged due to the TBI is reduced by a method of treating a TBI in a subject in need thereof according to aspects of the present disclosure which includes delivering a therapeutically effective amount of NeuroD1 to glial cells of the subject.

Signs and/or symptoms of TBI in a subject include presence of reactive astrocytes in the region of the brain damaged due to the TBI. The number of reactive astrocytes in the region of the brain damaged due to the TBI is reduced by a method of treating a TBI in a subject in need thereof according to aspects of the present disclosure which includes delivering a therapeutically effective amount of NeuroD1 to glial cells of the subject.

The therapeutically effective amount of NeuroD1 in the glial cells results in a greater number of functional neurons in the subject having a TBI, compared to an untreated subject having a TBI, whereby the TBI is treated. According to aspects of the present disclosure, a therapeutically effective amount of NeuroD1 in the glial cells results in a greater number of functional neurons in an area of the brain of the subject affected by TBI, compared to an untreated subject having a TBI, whereby the TBI is treated.

The subject in need of treatment may be human or non-human mammalian, but can be non-mammalian as well. Thus, the term “subject” refers to humans, and also to non-human mammals such as, but not limited to, non-human primates, cats, dogs, sheep, goats, horses, cows, pigs and rodents, such as but not limited to, mice and rats; as well as non-mammalian animals such as, but not limited to, birds, poultry, reptiles, amphibians. According to aspects of the present disclosure, the subject is human.

According to aspects of the present disclosure, the subject is a male. According to aspects of the present disclosure, the subject is a female. According to aspects of the present disclosure, the subject is gender neutral. According to aspects of the present disclosure, the subject is a premature newborn. According to aspects of the present disclosure, a premature newborn is born before 36 weeks gestation. According to aspects of the present disclosure, the subject is a term newborn. According to aspects of the present disclosure, a term newborn is below about 2 months old. According to aspects of the present disclosure, the subject is a neonate. According to aspects of the present disclosure, the subject is a neonate is below about 1 month old. According to aspects of the present disclosure, the subject is an infant. According to aspects of the present disclosure, an infant is between 2 months and 24 months old. According to aspects of the present disclosure, an infant is between 2 months and 3 months, between 2 months and 4 months, between 2 months and 5 months, between 3 months and 4 months, between 3 months and 5 months, between 3 months and 6 months, between 4 months and 5 months, between 4 months and 6 months, between 4 months and 7 months, between 5 months and 6 months, between 5 months and 7 months, between 5 months and 8 months, between 6 months and 7 months, between 6 months and 8 months, between 6 months and 9 months, between 7 months and 8 months, between 7 months and 9 months, between 7 months and 10 months, between 8 months and 9 months, between 8 months and 10 months, between 8 months and 11 months, between 9 months and 10 months, between 9 months and 11 months, between 9 months and 12 months, between 10 months and 11 months, between 10 months and 12 months, between 10 months and 13 months, between 11 months and 12 months, between 11 months and 13 months, between 11 months and 14 months, between 12 months and 13 months, between 12 months and 14 months, between 12 months and 15 months, between 13 months and 14 months, between 13 months and 15 months, between 13 months and 16 months, between 14 months and 15 months, between 14 months and 16 months, between 14 months and 17 months, between 15 months and 16 months, between 15 months and 17 months, between 15 months and 18 months, between 16 months and 17 months, between 16 months and 18 months, between 16 months and 19 months, between 17 months and 18 months, between 17 months and 19 months, between 17 months and 20 months, between 18 months and 19 months, between 18 months and 20 months, between 18 months and 21 months, between 19 months and 20 months, between 19 months and 21 months, between 19 months and 22 months, between 20 months and 21 months, between 20 months and 22 months, between 20 months and 23 months, between 21 months and 22 months, between 21 months and 23 months, between 21 months and 24 months, between 22 months and 23 months, between 22 months and 24 months, and between 23 months and 24 months old. According to aspects of the present disclosure, the subject is a toddler. According to aspects of the present disclosure, a toddler is between 1 year and 4 years old. According to aspects of the present disclosure, a toddler is between 1 year and 2 years, between 1 year and 3 years, between 1 year and 4 years, between 2 years and 3 years, between 2 years and 4 years, and between 3 years and 4 years old. According to aspects of the present disclosure, the subject is a young child. According to aspects of the present disclosure, a young child is between 2 years and 5 years old. According to aspects of the present disclosure, a young child is between 2 years and 3 years, between 2 years and 4 years, between 2 years and 5 years, between 3 years and 4 years, between 3 years and 5 years, and between 4 years and 5 years old. According to aspects of the present disclosure, the subject is a child. According to aspects of the present disclosure, a child is between 6 years and 12 years old. According to aspects of the present disclosure, a child is between 6 years and 7 years, between 6 years and 8 years, between 6 years and 9 years, between 7 years and 8 years, between 7 years and 9 years, between 7 years and 10 years, between 8 years and 9 years, between 8 years and 10 years, between 8 years and 11 years, between 9 years and 10 years, between 9 years and 11 years, between 9 years and 12 years, between 10 years and 11 years, between 10 years and 12 years, and between 11 years and 12 years old. According to aspects of the present disclosure, the subject is an adolescent. According to aspects of the present disclosure, an adolescent is between 13 years and 19 years old. According to aspects of the present disclosure, an adolescent is between 13 years and 14 years, between 13 years and 15 years, between 13 years and 16 years, between 14 years and 15 years, between 14 years and 16 years, between 14 years and 17 years, between 15 years and 16 years, between 15 years and 17 years, between 15 years and 18 years, between 16 years and 17 years, between 16 years and 18 years, between 16 years and 19 years, between 17 years and 18 years, between 17 years and 19 years, and between 18 years and 19 years old. According to aspects of the present disclosure, the subject is a pediatric subject. According to aspects of the present disclosure, a pediatric subject between 1 day and 18 years old. According to aspects of the present disclosure, a pediatric subject is between 1 day and 1 year, between 1 day and 2 years, between 1 day and 3 years, between 1 year and 2 years, between 1 year and 3 years, between 1 year and 4 years, between 2 years and 3 years, between 2 years and 4 years, between 2 years and 5 years, between 3 years and 4 years, between 3 years and 5 years, between 3 years and 6 years, between 4 years and 5 years, between 4 years and 6 years, between 4 years and 7 years, between 5 years and 6 years, between 5 years and 7 years, between 5 years and 8 years, between 6 years and 7 years, between 6 years and 8 years, between 6 years and 9 years, between 7 years and 8 years, between 7 years and 9 years, between 7 years and 10 years, between 8 years and 9 years, between 8 years and 10 years, between 8 years and 11 years, between 9 years and 10 years, between 9 years and 11 years, between 9 years and 12 years, between 10 years and 11 years, between 10 years and 12 years, between 10 years and 13 years, between 11 years and 12 years, between 11 years and 13 years, between 11 years and 14 years, between 12 years and 13 years, between 12 years and 14 years, between 12 years and 15 years, between 13 years and 14 years, between 13 years and 15 years, between 13 years and 16 years, between 14 years and 15 years, between 14 years and 16 years, between 14 years and 17 years, between 15 years and 16 years, between 15 years and 17 years, between 15 years and 18 years, between 16 years and 17 years, between 16 years and 18 years, and between 17 years and 18 years old. According to aspects of the present disclosure, the subject is a geriatric subject. According to aspects of the present disclosure, a geriatric subject is between 65 years and 95 or more years old. According to aspects of the present disclosure, a geriatric subject is between 65 years and 70 years, between 65 years and 75 years, between 65 years and 80 years, between 70 years and 75 years, between 70 years and 80 years, between 70 years and 85 years, between 75 years and 80 years, between 75 years and 85 years, between 75 years and 90 years, between 80 years and 85 years, between 80 years and 90 years, between 80 years and 95 years, between 85 years and 90 years, and between 85 years and 95 years old. In one aspect, a subject in need thereof is an adult. According to aspects of the present disclosure, an adult subject is between 20 years and 95 or more years old. According to aspects of the present disclosure, an adult subject is between 20 years and 25 years, between 20 years and 30 years, between 20 years and 35 years, between 25 years and 30 years, between 25 years and 35 years, between 25 years and 40 years, between 30 years and 35 years, between 30 years and 40 years, between 30 years and 45 years, between 35 years and 40 years, between 35 years and 45 years, between 35 years and 50 years, between 40 years and 45 years, between 40 years and 50 years, between 40 years and 55 years, between 45 years and 50 years, between 45 years and 55 years, between 45 years and 60 years, between 50 years and 55 years, between 50 years and 60 years, between 50 years and 65 years, between 55 years and 60 years, between 55 years and 65 years, between 55 years and 70 years, between 60 years and 65 years, between 60 years and 70 years, between 60 years and 75 years, between 65 years and 70 years, between 65 years and 75 years, between 65 years and 80 years, between 70 years and 75 years, between 70 years and 80 years, between 70 years and 85 years, between 75 years and 80 years, between 75 years and 85 years, between 75 years and 90 years, between 80 years and 85 years, between 80 years and 90 years, between 80 years and 95 years, between 85 years and 90 years, and between 85 years and 95 years old. According to aspects of the present disclosure, a subject is between 1 year and 5 years, between 2 years and 10 years, between 3 years and 18 years, between 21 years and 50 years, between 21 years and 40 years, between 21 years and 30 years, between 50 years and 90 years, between 60 years and 90 years, between 70 years and 90 years, between 60 years and 80 years, or between 65 years and 75 years old. According to aspects of the present disclosure, a subject is a young old subject (65 to 74 years old). According to aspects of the present disclosure, a subject is a middle old subject (75 to 84 years old). According to aspects of the present disclosure, a subject is an old subject (>85 years old).

Methods of treatment of TBI in a subject include administration of a therapeutically effective amount of NeuroD1 to the subject in the local region of the TBI, at or near the location the brain injury site, according to aspects of the present disclosure.

Methods of treatment of TBI in a subject include administration of a therapeutically effective amount of NeuroD1 to the subject in the local region of the TBI, in or near a glial scar caused by the TBI, according to aspects of the present disclosure.

Methods of treatment of TBI in a subject include administration of a therapeutically effective amount of NeuroD1 to the subject in the local region of the TBI, in or near a region of gliosis, particularly astrogliosis and/or microgliosis, according to aspects of the present disclosure.

The term “gliosis” includes “astrogliosis” and “microgliosis” and refers to an increase in astrocytes and reactive astrocytes, i.e. astrogiosis, and an increase in microglia and hypertrophic microglia, i.e. microgliosis, due to brain damage. Without being limited by any scientific theory, gliosis is believed to be a protective reaction of glial cells in response to brain damage, providing beneficial effects such as insulating the injury area, removing debris of dead cells, and protecting the remaining healthy cells. However, gliosis can impede neural regeneration and produce negative effects on the local microenvironment, leading to further neurodegeneration. Thus, conversion of glial cells into functional neurons, where the glial cells are involved in gliosis by expressing exogenous NeuroD1 in the glial cells provides beneficial outcomes in the treatment of TBI. According to aspects of the present disclosure, non-limiting examples of beneficial outcomes include regeneration of functional neurons to replace, or at least partially replace, the neurons lost due to TBI, reduction in the number of reactive astrocytes by conversion of the reactive astrocytes to functional neurons thereby modulating the negative effects of gliosis, repair of the damaged neural network caused by the TBI, and rebalancing the microenvironment disrupted by the TBI.

According to aspects of the present disclosure, administration of a therapeutically effective amount of NeuroD1 ameliorates the effects of TBI in a subject in need thereof. According to aspects of the present disclosures, administration of a therapeutically effective amount of NeuroD1 has enhanced effects when administered to reactive astrocytes compared to quiescent astrocytes. According to aspects of the present disclosure, administration of a therapeutically effective amount of NeuroD1 can be between 3 days to 60 days, between 5 days to 45 days, between 8 days to 30 days following the TBI in the subject. According to aspects of the present disclosure, administration can be 2 days to 1 year or later following the TBI in the subject. According to aspects of the present disclosure, administration of a therapeutically effective amount of NeuroD1 can be between 3 days and 5 days, between 3 days and 10 days, between 3 days and 15 days, between 5 days and 10 days, between 5 days and 15 days, between 5 days and 20 days, between 10 days and 15 days, between 10 days and 20 days, between 10 days and 25 days, between 15 days and 20 days, between 15 days and 25 days, between 15 days and 30 days, between 20 days and 25 days, between 20 days and 30 days, between 20 days and 35 days, between 25 days and 30 days, between 25 days and 35 days, between 25 days and 40 days, between 30 days and 35 days, between 30 days and 40 days, between 30 days and 45 days, between 35 days and 40 days, between 35 days and 45 days, between 35 days and 50 days, between 40 days and 45 days, between 40 days and 50 days, between 40 days and 55 days, between 45 days and 50 days, between 45 days and 55 days, between 45 days and 60 days, between 50 days and 55 days, between 50 days and 60 days, or between 55 days and 60 days. According to aspects of the present disclosure, administration of a therapeutically effective amount of NeuroD1 can be between 5 days and 10 days, between 5 days and 15 days, between 5 days and 20 days, 10 days and 15 days, between 10 days and 20 days, between 10 days and 25 days, between 15 days and 20 days, between 15 days and 25 days, between 15 days and 30 days, between 20 days and 25 days, between 20 days and 30 days, between 20 days and 35 days, between 25 days and 30 days, between 25 days and 35 days, between 25 days and 40 days, between 30 days and 35 days, between 30 days and 40 days, between 30 days and 45 days, between 35 days and 40 days, between 35 days and 45 days, or between 40 days and 45 days. According to aspects of the present disclosure, administration of a therapeutically effective amount of NeuroD1 can be between 8 days and 10 days, between 8 days and 15 days, between 8 days and 20 days, 10 days and 15 days, between 10 days and 20 days, between 10 days and 25 days, between 15 days and 20 days, between 15 days and 25 days, between 15 days and 30 days, between 20 days and 25 days, between 20 days and 30 days, or between 25 days and 30 days.

According to aspects of the present disclosure, providing the exogenous NeuroD1 includes providing exogenous NeuroD1 to the at least one reactive astrocyte at a first treatment time in the range of about 1 day to about 10 days after the TBI. According to aspects of the present disclosure, exogenous NeuroD1 is provided to the at least one reactive astrocyte between 1 day and 2 days, between 1 day and 3 days, between 1 day and 4 days, between 2 days and 3 days, between 2 days and 4 days, between 2 days and 5 days, between 3 days and 4 days, between 3 days and 5 days, between 3 days and 6 days, between 4 days and 5 days, between 4 days and 6 days, between 4 days and 7 days, between 5 days and 6 days, between 5 days and 7 days, between 5 days and 8 days, between 6 days and 7 days, between 6 days and 8 days, between 6 days and 9 days, between 7 days and 8 days, between 7 days and 9 days, between 7 days and 10 days, between 8 days and 9 days, between 8 days and 10 days, or between 9 days and 10 days. According to aspects of the present disclosure, exogenous NeuroD1 is provided to the at least one reactive astrocyte at a treatment time of 1 day after the TBI. According to aspects of the present disclosure, exogenous NeuroD1 is provided to the at least one reactive astrocyte at a treatment time of 2 days after the TBI. According to aspects of the present disclosure, exogenous NeuroD1 is provided to the at least one reactive astrocyte at a treatment time of 3 days after the TBI. According to aspects of the present disclosure, exogenous NeuroD1 is provided to the at least one reactive astrocyte at a treatment time of 4 days after the TBI. According to aspects of the present disclosure, exogenous NeuroD1 is provided to the at least one reactive astrocyte at a treatment time of 5 days after the TBI. According to aspects of the present disclosure, exogenous NeuroD1 is provided to the at least one reactive astrocyte at a treatment time of 6 days after the TBI. According to aspects of the present disclosure, exogenous NeuroD1 is provided to the at least one reactive astrocyte at a treatment time of 7 days after the TBI. According to aspects of the present disclosure, exogenous NeuroD1 is provided to the at least one reactive astrocyte at a treatment time of 8 days after the TBI. According to aspects of the present disclosure, exogenous NeuroD1 is provided to the at least one reactive astrocyte at a treatment time of 9 days after the TBI. According to aspects of the present disclosure, exogenous NeuroD1 is provided to the at least one reactive astrocyte at a treatment time of 10 days after the TBI.

According to aspects of the present disclosure, the TBI causes a period of astrogliosis in the damaged region, and providing the exogenous NeuroD1 includes providing exogenous NeuroD1 to the at least one reactive astrocyte at a first treatment time during the period of astrogliosis or within 4 weeks after the period of astrogliosis. According to aspects of the present disclosure, the exogenous NeuroD1 is provided to the at least one reactive astrocyte at a second treatment time after the first treatment time and during the period of astrogliosis or within 4 weeks after the period of astrogliosis. According to aspects of the present disclosure, the exogenous NeuroD1 is provided to the at least one reactive astrocyte at a third treatment time after the second treatment time and during the period of astrogliosis or within 4 weeks after the period of astrogliosis. More than three treatments are optionally provided, such as a fourth treatment at a fourth treatment time after the third treatment, a fifth treatment at a fifth treatment time after the fourth treatment, and so on relating to sixth, seventh, eighth, ninth, and tenth, or more, treatments including administration of exogenous NeuroD1, during the period of astrogliosis or within 4 weeks after the period of astrogliosis.

Combinations of therapies treating TBI in a subject are administered according to aspects of the present disclosure.

According to particular aspects an additional pharmaceutical agent or therapeutic treatment administered to a subject to treat TBI in an individual subject in need thereof include treatments such as, but not limited to, repairing a skull fracture, removing a blood clot, relieving pressure inside the skull, administration of one or more anti-inflammation agents, administration of one or more anti-anxiety agents, and administration of one or more anti-coagulant agents, administration of one or more anticonvulsants, administration of one or more antidepressants, administration of one or more muscle relaxants, physical therapy, speech therapy, and cognitive therapy.

According to aspects of the present disclosure, NeuroD1 treatment is administered to a subject having a TBI as diagnosed and/or assessed by a medical examination. The term “medical examination” as used herein refers to any examination of a subject effective to diagnose or assess the subject for putative TBI, including neurological examination and physical examination.

According to aspects of the present disclosure, the medical examination includes an imaging technique and/or an electrophysiological technique and NeuroD1 treatment is administered to a subject having a TBI as diagnosed and/or assessed by an imaging technique and/or an electrophysiological technique.

Electrophysiology techniques, such as electroencephalography (EEG), can be used to assess functional changes in neural firing caused by neuronal cell death or injury due to TBI.

Imaging techniques such as magnetic resonance imaging (MM), fMRI, Near Infrared Spectroscopy, position emission tomography (PET) scan, computerized axial tomography (CAT) scan, and ultrasound, can be used to assess structural and/or functional changes caused by neuronal cell death or injury due to TBI.

According to aspects of the present disclosure, presence of non-functional neurons due to TBI are detected by a functional assay, such as fMRI.

The term “fMRI” refers to functional magnetic resonance imaging, an imaging procedure that detects and measure brain activity by detecting associated changes in blood flow.

Methods of medical examination may be used singularly, or in any combination, to diagnose and/or assess a TBI in the subject.

Moreover, methods of medical examination may be used singularly, or in any combination, to assess efficacy of NeuroD1 treatment of a TBI in the subject.

According to aspects of the present disclosure, NeuroD1 treatment of a subject is monitored during or after treatment to monitor progress and/or final outcome of the treatment. Post-treatment assay for successful functional neuron integration and restoration of tissue microenvironment is diagnosed by restoration or near-restoration of normal electrophysiology, brain tissue structure, and neuronal function. Non-invasive methods to assay neuronal function include EEG. Neuronal function may be non-invasively assayed via Near Infrared Spectroscopy and fMRI.

Non-invasive methods to assay brain tissue structure include MRI, CAT scan, PET scan, or ultrasound.

Behavioral assays may be used to non-invasively assay for restoration of brain function following TBI. The behavioral assay should be matched to the loss of function caused by the TBI. For example, if the TBI caused paralysis, the patient's mobility and limb dexterity should be tested. If the TBI caused loss or slowing of speech, patient's ability to communicate via spoken word should be assayed. Restoration of normal behavior post-NeuroD1 treatment indicates successful creation and integration of effective neuronal circuits. These methods may be used singularly or in any combination to assay for neuronal function and brain tissue health. Assays to evaluate treatment with NeuroD1 may be performed at any point, such as 1 day, 2 days, 3 days, one week, 2 weeks, 3 weeks, one month, or later, after NeuroD1 treatment. Such assays may be performed prior to NeuroD1 treatment in order to establish a baseline comparison if desired.

In particular aspects according to the present disclosure, NeuroD1 is administered at the periphery of the injury site where a glial scar will develop if the subject is untreated or where a glial scar is already present. Glial scar location may be determined by assaying tissue structure or function. As described above, non-invasive methods to assay structural and/or functional changes caused by TBI including MRI, fMRT, CAT scan, or ultrasound. Functional assay may include EEG recording and/or fMRT.

In particular aspects according to the present disclosure, NeuroD1 is administered as an expression vector containing a nucleic acid sequence encoding NeuroD1. According to aspects of the present disclosure, an expression vector containing a nucleic acid sequence encoding NeuroD1 is delivered by injection, into the brain of a subject. According to aspects of the present disclosure, an expression vector containing a nucleic acid sequence encoding NeuroD1 is delivered by stereotactic injection, into the brain of a subject.

According to aspects of the present disclosure, a viral vector including a nucleic acid encoding NeuroD1 is delivered by injection into the central or peripheral nerve tissue of a subject. According to aspects of the present disclosure injection into the central or peripheral nerve tissue is selected from the group consisting of intracerebral injection, spinal cord injection, injection into the cerebrospinal fluid, and injection into the peripheral nerve ganglia. Alternative viral delivery methods include but not limited to intravenous injection, intranasal infusion, intramuscle injection, intrathecal injection, and intraperitoneal injection.

According to aspects of the present disclosure, a viral vector including a nucleic acid encoding NeuroD1 is delivered by injection into the brain of a subject. According to aspects of the present disclosure, a viral vector including a nucleic acid encoding NeuroD1 is delivered by stereotactic injection into the brain of a subject.

Method and compositions for treating a neurological condition in a subject in need thereof are provided according to aspects of the present disclosure which include providing a viral vector comprising a nucleic acid encoding NeuroD1; and delivering the viral vector to the brain of the subject, whereby the viral vector infects glial cells of the brain producing infected glial cells and whereby exogenous NeuroD1 is expressed in the infected glial cells at a therapeutically effective level, wherein the expression of NeuroD1 in the infected cells results in a greater number of functional neurons in the subject with a TBI compared to an untreated subject having a TBI, whereby the TBI is treated. In addition to the generation of new functional neurons, the number of reactive glial cells is reduced, resulting in fewer neuroinhibitory factors released, less neuroinflammation, more blood vessels that are also evenly distributed, thereby making local environment more permissive to neuronal growth or axon penetration, hence alleviating at least one sign and/or symptom of TBI.

Adeno-associated virus (AAV) vectors are particularly useful in methods according to aspects of the present disclosure and will infect both dividing and non-dividing cells, at an injection site. AAV are ubiquitous, noncytopathic, replication-incompetent members of ssDNA animal virus of parvoviridae family. As used herein, an “AAV vector” refers to an AAV packaged with a DNA vector construct. According to aspects of the present disclosure, an AAV vector is selected from the group consisting of AAV vector serotype 1, AAV vector serotype 2, AAV vector serotype 3, AAV vector serotype 4, AAV vector serotype 5, AAV vector serotype 6, AAV vector serotype 7, AAV vector serotype 8, AAV vector serotype 9, AAV vector serotype 10, AAV vector serotype 11, and AAV vector serotype 12. According to aspects of the present disclosure, an AAV vector is selected from the group consisting AAV serotype 2, AAV serotype 5, and AAV serotype 9. According to aspects of the present disclosure, an AAV vector is AAV serotype 1. According to aspects of the present disclosure, an AAV vector is AAV serotype 2. According to aspects of the present disclosure, an AAV vector is AAV serotype 3. According to aspects of the present disclosure, an AAV vector is AAV serotype 4. According to aspects of the present disclosure, an AAV vector is AAV serotype 5. In one aspect, According to aspects of the present disclosure, an AAV vector is AAV serotype 6. According to aspects of the present disclosure, an AAV vector is AAV serotype 7. According to aspects of the present disclosure, an AAV vector is AAV serotype 8. According to aspects of the present disclosure, an AAV vector is AAV serotype 9. According to aspects of the present disclosure, an AAV vector is AAV serotype 10. According to aspects of the present disclosure, an AAV vector is AAV serotype 11. According to aspects of the present disclosure, an AAV vector is AAV serotype 12.

A “FLEX” switch approach is used to express NeuroD1 in infected cells according to aspects of the present disclosure. The terms “FLEX” and “flip-excision” are used interchangeably to indicate a method in which two pairs of heterotypic, antiparallel loxP-type recombination sites are disposed on either side of an inverted NeuroD1 coding sequence which first undergo an inversion of the coding sequence followed by excision of two sites, leading to one of each orthogonal recombination site oppositely oriented and incapable of further recombination, achieving stable inversion, see for example Schnutgen et al., Nature Biotechnology 21:562-565, 2003; and Atasoy et al, J. Neurosci., 28:7025-7030, 2008. Since the site-specific recombinase under control of a glial cell-specific promoter will be strongly expressed in glial cells, including reactive astrocytes, NeuroD1 will also be expressed in glial cells, including reactive astrocytes. Then, when the stop codon in front of NeuroD1 is removed from recombination, the constitutive or neuron-specific promoter will drive high expression of NeuroD1, allowing reactive astrocytes to be converted into functional neurons.

According to particular aspects of the present disclosure, NeuroD1 is administered to a subject in need thereof by administration of 1) an adeno-associated virus expression vector including a DNA sequence encoding a site-specific recombinase under transcriptional control of an astrocyte-specific promoter such as GFAP or S100b or Aldh1L1; and 2) an adeno-associated virus expression vector including a DNA sequence encoding NeuroD1 under transcriptional control of a ubiquitous (constitutive) promoter or a neuron-specific promoter wherein the DNA sequence encoding NeuroD1 is inverted and in the wrong orientation for expression of NeuroD1 until the site-specific recombinase inverts the inverted DNA sequence encoding NeuroD1, thereby allowing expression of NeuroD1.

Site-specific recombinases and their recognition sites include, for example, Cre recombinase along with recognition sites loxP and lox2272 sites, or FLP-FRT recombination, or their combinations.

As used herein, the term “AAV particle” refers to packaged capsid forms of the AAV virus that transmits its nucleic acid genome to cells.

In order to achieve optimal infection, a concentration of 10¹⁰-10¹⁴ AAV particles/ml, 1-1000 μl of volume, is injected at a controlled flow rate of 0.1-5.0 μl/minute. According to aspects of the present disclosure, a concentration between 10¹⁰ AAV particles/mL and 10¹¹ AAV particles/mL, between 10¹⁰ AAV particles/mL and 10¹² AAV particles/mL, between 10¹⁰ AAV particles/mL and 10¹³ AAV particles/mL, between 10¹¹ AAV particles/mL and 10¹² AAV particles/mL, between 10¹¹ AAV particles/mL and 10¹³ AAV particles/mL, between 10¹¹ AAV particles/mL and 10¹⁴ AAV particles/mL, between 10¹² AAV particles/mL and 10¹³ AAV particles/mL, between 10¹² AAV particles/mL and 10¹⁴ AAV particles/mL, or between 10¹³ AAV particles/mL and 10¹⁴ AAV particles/mL is injected. According to aspects of the present disclosure, an AAV particle is injected at a volume between 1 μL and 100 between 1 μL and 200 μL, between 1 μL and 300 μL, between 100 μL and 200 μL, between 100 μL and 300 μL, between 100 μL and 400 μL, between 200 μL and 300 μL, between 200 μL and 400 μL, between 200 μL and 500 μL, between 300 μL and 400 μL, between 300 μL and 500 μL, between 300 μL and 600 μL, between 400 μL and 500 μL, between 400 μL and 600 μL, between 400 uL and 700 μL, between 500 μL and 600 μL, between 500 μL and 700 μL, between 500 μL and 800 μL, between 600 μL and 700 μL, between 600 μL and 800 μL, between 600 μL and 900 μL, between 700 μL and 800 μL, between 700 μL and 900 μL, between 700 μL and 1000 μL, between 800 μL and 900 μL, between 800 μL and 1000 μL, or between 900 μL and 1000 According to aspects of the present disclosure, the flow rate is between 0.1 μL/minute and 0.2 μL/minute, between 0.1 μL/minute and 0.3 μL/minute, between 0.1 μL/minute and 0.4 μL/minute, between 0.2 μL/minute and 0.3 μL/minute, between 0.2 μL/minute and 0.4 μL/minute, between 0.2 μL/minute and 0.5 μL/minute, between 0.3 μL/minute and 0.4 μL/minute, between 0.3 μL/minute and 0.5 μL/minute, between 0.3 μL/minute and 0.6 μL/minute, between 0.4 μL/minute and 0.5 μL/minute, between 0.4 μL/minute and 0.6 μL/minute, between 0.4 μL/minute and 0.7 μL/minute, between 0.5 μL/minute and 0.6 μL/minute, between 0.5 μL/minute and 0.7 μL/minute, between 0.5 μL/minute and 0.8 μL/minute, between 0.6 μL/minute and 0.7 μL/minute, between 0.6 μL/minute and 0.8 μL/minute, between 0.6 μL/minute and 0.9 μL/minute, between 0.7 μL/minute and 0.8 μL/minute, between 0.7 μL/minute and 0.9 μL/minute, between 0.7 μL/minute and 1.0 μL/minute, between 0.8 μL/minute and 0.9 μL/minute, between 0.8 μL/minute and 1.0 μL/minute, between 0.8 μL/minute and 1.1 μL/minute, between 0.9 μL/minute and 1.0 μL/minute, between 0.9 μL/minute and 1.1 μL/minute, between 0.9 μL/minute and 1.2 μL/minute, between 1.0 μL/minute and 1.1 μL/minute, between 1.0 μL/minute and 1.2 μL/minute, between 1.0 μL/minute and 1.3 μL/minute, between 1.1 μL/minute and 1.2 μL/minute, between 1.1 μL/minute and 1.3 μL/minute, between 1.1 μL/minute and 1.4 μL/minute, between 1.2 μL/minute and 1.3 μL/minute, between 1.2 μL/minute and 1.4 μL/minute, between 1.2 μL/minute and 1.5 μL/minute, between 1.3 μL/minute and 1.4 μL/minute, between 1.3 μL/minute and 1.5 μL/minute, between 1.3 μL/minute and 1.6 μL/minute, between 1.4 μL/minute and 1.5 μL/minute, between 1.4 μL/minute and 1.6 μL/minute, between 1.4 μL/minute and 1.7 μL/minute, between 1.5 μL/minute and 1.6 μL/minute, between 1.5 μL/minute and 1.7 μL/minute, between 1.5 μL/minute and 1.8 μL/minute, between 1.6 μL/minute and 1.7 μL/minute, between 1.6 μL/minute and 1.8 μL/minute, between 1.6 μL/minute and 1.9 μL/minute, between 1.7 μL/minute and 1.8 μL/minute, between 1.7 μL/minute and 1.9 μL/minute, between 1.7 μL/minute and 2.0 μL/minute, between 1.8 μL/minute and 1.9 μL/minute, between 1.8 μL/minute and 2.0 μL/minute, between 1.8 μL/minute and 2.1 μL/minute, between 1.9 μL/minute and 2.0 μL/minute, between 1.9 μL/minute and 2.1 μL/minute, between 1.9 μL/minute and 2.2 μL/minute, between 2.0 μL/minute and 2.1 μL/minute, between 2.0 μL/minute and 2.2 μL/minute, between 2.0 μL/minute and 2.3 μL/minute, between 2.1 μL/minute and 2.2 μL/minute, between 2.1 μL/minute and 2.3 μL/minute, between 2.1 μL/minute and 2.4 μL/minute, between 2.2 μL/minute and 2.3 μL/minute, between 2.2 μL/minute and 2.4 μL/minute, between 2.2 μL/minute and 2.5 μL/minute, between 2.3 μL/minute and 2.4 μL/minute, between 2.3 μL/minute and 2.5 μL/minute, between 2.3 μL/minute and 2.6 μL/minute, between 2.4 μL/minute and 2.5 μL/minute, between 2.4 μL/minute and 2.6 μL/minute, between 2.4 μL/minute and 2.7 μL/minute, between 2.5 μL/minute and 2.6 μL/minute, between 2.5 μL/minute and 2.7 μL/minute, between 2.5 μL/minute and 2.8 μL/minute, between 2.6 μL/minute and 2.7 μL/minute, between 2.6 μL/minute and 2.8 μL/minute, between 2.6 μL/minute and 2.9 μL/minute, between 2.7 μL/minute and 2.8 μL/minute, between 2.7 μL/minute and 2.9 μL/minute, between 2.7 μL/minute and 3.0 μL/minute, between 2.8 μL/minute and 2.9 μL/minute, between 2.8 μL/minute and 3.0 μL/minute, between 2.8 μL/minute and 3.1 μL/minute, between 2.9 μL/minute and 3.0 μL/minute, between 2.9 μL/minute and 3.1 μL/minute, between 2.9 μL/minute and 3.2 μL/minute, between 3.0 μL/minute and 3.1 μL/minute, between 3.0 μL/minute and 3.2 μL/minute, between 3.0 μL/minute and 3.3 μL/minute, between 3.1 μL/minute and 3.2 μL/minute, between 3.1 μL/minute and 3.3 μL/minute, between 3.1 μL/minute and 3.4 μL/minute, between 3.2 μL/minute and 3.3 μL/minute, between 3.2 μL/minute and 3.4 μL/minute, between 3.2 μL/minute and 3.5 μL/minute, between 3.3 μL/minute and 3.4 μL/minute, between 3.3 μL/minute and 3.5 μL/minute, between 3.3 μL/minute and 3.6 μL/minute, between 3.4 μL/minute and 3.5 μL/minute, between 3.4 μL/minute and 3.6 μL/minute, between 3.4 μL/minute and 3.7 μL/minute, between 3.5 μL/minute and 3.6 μL/minute, between 3.5 μL/minute and 3.7 μL/minute, between 3.5 μL/minute and 3.8 μL/minute, between 3.6 μL/minute and 3.7 μL/minute, between 3.6 μL/minute and 3.8 μL/minute, between 3.6 μL/minute and 3.9 μL/minute, between 3.7 μL/minute and 3.8 μL/minute, between 3.7 μL/minute and 3.9 μL/minute, between 3.7 μL/minute and 4.0 μL/minute, between 3.8 μL/minute and 3.9 μL/minute, between 3.8 μL/minute and 4.0 μL/minute, between 3.8 μL/minute and 4.1 μL/minute, between 3.9 μL/minute and 4.0 μL/minute, between 3.9 μL/minute and 4.1 μL/minute, between 3.9 μL/minute and 4.2 μL/minute, between 4.0 μL/minute and 4.1 μL/minute, between 4.0 μL/minute and 4.2 μL/minute, between 4.0 μL/minute and 4.3 μL/minute, between 4.1 μL/minute and 4.2 μL/minute, between 4.1 μL/minute and 4.3 μL/minute, between 4.1 μL/minute and 4.4 μL/minute, between 4.2 μL/minute and 4.3 μL/minute, between 4.2 μL/minute and 4.4 μL/minute, between 4.2 μL/minute and 4.5 μL/minute, between 4.3 μL/minute and 4.4 μL/minute, between 4.3 μL/minute and 4.5 μL/minute, between 4.3 μL/minute and 4.6 μL/minute, between 4.4 μL/minute and 4.5 μL/minute, between 4.4 μL/minute and 4.6 μL/minute, between 4.4 μL/minute and 4.7 μL/minute, between 4.5 μL/minute and 4.6 μL/minute, between 4.5 μL/minute and 4.7 μL/minute, between 4.5 μL/minute and 4.8 μL/minute, between 4.6 μL/minute and 4.7 μL/minute, between 4.6 μL/minute and 4.8 μL/minute, between 4.6 μL/minute and 4.9 μL/minute, between 4.7 μL/minute and 4.8 μL/minute, between 4.7 μL/minute and 4.9 μL/minute, between 4.7 μL/minute and 5.0 μL/minute, 4.8 μL/minute and 4.9 μL/minute, between 4.8 μL/minute and 5.0 μL/minute, or between 4.9 μL/minute and 5.0 μL/minute.

According to aspects of the present disclosure, an AAV vector including a nucleic acid encoding NeuroD1 under transcriptional control of a ubiquitous (constitutive) promoter or a neuron-specific promoter wherein the DNA sequence encoding NeuroD1 is inverted and in the wrong orientation for expression of NeuroD1 and further includes sites for recombinase activity by a site specific recombinase, until the site-specific recombinase inverts the inverted DNA sequence encoding NeuroD1, thereby allowing expression of NeuroD1, is delivered by stereotactic injection into the brain of a subject along with an AAV encoding a site specific recombinase.

According to aspects of the present disclosure, an AAV vector including a nucleic acid encoding NeuroD1 under transcriptional control of a ubiquitous (constitutive) promoter or a neuron-specific promoter wherein the DNA sequence encoding NeuroD1 is inverted and in the wrong orientation for expression of NeuroD1 and further includes sites for recombinase activity by a site specific recombinase, until the site-specific recombinase inverts the inverted DNA sequence encoding NeuroD1, thereby allowing expression of NeuroD1, is delivered by stereotactic injection into the brain of a subject along with an adeno-associated virus encoding a site specific recombinase in the region of or at the site of disruption of normal blood flow in the central nervous system (CNS) according to aspects of the present disclosure. Optionally, the site of stereotactic injection is in or near a glial scar caused by disruption of normal blood flow in the CNS.

According to aspects of the present disclosure, a composition comprises a first recombinant expression vector comprising a glial cell specific promoter operably linked to a nucleic acid encoding a site specific recombinase and a second recombinant expression vector comprising a promoter operably linked to a nucleic acid sequence encoding NeuroD1, a nucleic acid sequence encoding a reporter gene, an enhancer, and a regulatory element.

According to aspects of the present disclosure, a composition comprises a first recombinant AAV expression vector comprising a glial cell specific promoter operably linked to a nucleic acid encoding a site specific recombinase and a second recombinant AAV expression vector comprising a promoter operably linked to a nucleic acid sequence encoding NeuroD1, a nucleic acid sequence encoding a reporter gene, an enhancer, and a regulatory element.

According to aspects of the present disclosure, the site-specific recombinase is Cre recombinase and the sites for recombinase activity are recognition sites loxP and lox2272 sites.

The term “NeuroD1” encompasses human NeuroD1 protein, identified here as SEQ ID NO: 2 and mouse NeuroD1 protein, identified here as SEQ ID NO: 4. In addition to the NeuroD1 protein of SEQ ID NO: 2 and SEQ ID NO: 4, the term “NeuroD1” encompasses variants of NeuroD1 protein, such as variants of SEQ ID NO: 2 and SEQ ID NO: 4, which may be included in methods and compositions of the present disclosure. As used herein, the term “variant” refers to naturally occurring genetic variations and recombinantly prepared variations, each of which contain one or more changes in its amino acid sequence compared to a reference NeuroD1 protein, such as SEQ ID NO: 2 or SEQ ID NO: 4, wherein the variant retains the functional properties of the reference protein. Such changes include those in which one or more amino acid residues have been modified by amino acid substitution, addition or deletion. The term “variant” encompasses orthologs of human NeuroD1, including for example mammalian and bird NeuroD1, such as, but not limited to NeuroD1 orthologs from a non-human primate, cat, dog, sheep, goat, horse, cow, pig, bird, poultry animal and rodent such as but not limited to mouse and rat. In a non-limiting example, mouse NeuroD1, exemplified herein as amino acid sequence SEQ ID NO: 4 is an ortholog of human NeuroD1.

Preferred variants have at least 75%, 80%, 85%, 90%, 91%, 92%, 93%, 94%, 95%, 96%, 97%, 98%, or 99% identity to SEQ ID NO: 2 or SEQ ID NO: 4, wherein the variant retains the functional properties of the reference protein.

Mutations can be introduced using standard molecular biology techniques, such as site-directed mutagenesis and PCR-mediated mutagenesis. One of skill in the art will recognize that one or more amino acid mutations can be introduced without altering the functional properties of the NeuroD1 protein. For example, one or more amino acid substitutions, additions, or deletions can be made without altering the functional properties of the NeuroD1 protein of SEQ ID NO: 2 or 4.

Conservative amino acid substitutions can be made in a NeuroD1 protein to produce a NeuroD1 protein variant, wherein the variant retains the functional properties of the reference protein. Conservative amino acid substitutions are art recognized substitutions of one amino acid for another amino acid having similar characteristics. For example, each amino acid may be described as having one or more of the following characteristics: electropositive, electronegative, aliphatic, aromatic, polar, hydrophobic and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine and valine; aromatic amino acids include phenylalanine, glycine, tyrosine and tryptophan; polar amino acids include aspartate, glutamate, histidine, lysine, asparagine, glutamine, arginine, serine, threonine and tyrosine; and hydrophobic amino acids include alanine, cysteine, phenylalanine, glycine, isoleucine, leucine, methionine, proline, valine and tryptophan; and conservative substitutions include substitution among amino acids within each group. Amino acids may also be described in terms of relative size, alanine, cysteine, aspartate, glycine, asparagine, proline, threonine, serine, valine, all typically considered to be small.

NeuroD1 variants can include synthetic amino acid analogs, amino acid derivatives and/or non-standard amino acids, illustratively including, without limitation, alpha-aminobutyric acid, citrulline, canavanine, cyanoalanine, diaminobutyric acid, diaminopimelic acid, dihydroxy-phenylalanine, djenkolic acid, homoarginine, hydroxyproline, norleucine, norvaline, 3-phosphoserine, homoserine, 5-hydroxytryptophan, 1-methylhistidine, 3-methylhistidine, and ornithine.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, PNAS 87:2264 2268, modified as in Karlin and Altschul, 1993, PNAS. 90:5873 5877. Such an algorithm is incorporated into the NBLAST and)(BLAST programs of Altschul et al., 1990, J. Mol. Biol. 215:403. BLAST nucleotide searches are performed with the NBLAST nucleotide program parameters set, e.g., for score=100, wordlength=12 to obtain nucleotide sequences homologous to a nucleic acid molecules of the present disclosure.

BLAST protein searches are performed with the)(BLAST program parameters set, e.g., to score 50, wordlength=3 to obtain amino acid sequences homologous to a protein molecule of the present disclosure. To obtain gapped alignments for comparison purposes, Gapped BLAST are utilized as described in Altschul et al., 1997, Nucleic Acids Res. 25:3389 3402. Alternatively, PSI BLAST is used to perform an iterated search which detects distant relationships between molecules. When utilizing BLAST, Gapped BLAST, and PSI Blast programs, the default parameters of the respective programs (e.g., of XBLAST and NBLAST) are used (see, e.g., the NCBI website).

Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 is used.

The percent identity between two sequences is determined using techniques similar to those described above, with or without allowing gaps. In calculating percent identity, typically only exact matches are counted.

The term “NeuroD1 protein” encompasses fragments of the NeuroD1 protein, such as fragments of SEQ ID NOs. 2 and 4 and variants thereof, operable in methods and compositions of the present disclosure.

NeuroD1 proteins and nucleic acids may be isolated from natural sources, such as the brain of an organism or cells of a cell line which expresses NeuroD1. Alternatively, NeuroD1 protein or nucleic acid may be generated recombinantly, such as by expression using an expression construct, in vitro or in vivo. NeuroD1 proteins and nucleic acids may also be synthesized by well-known methods.

NeuroD1 included in methods and compositions of the present disclosure is preferably produced using recombinant nucleic acid technology. Recombinant NeuroD1 production includes introducing a recombinant expression vector encompassing a nucleic acid sequence, such as a DNA sequence or RNA sequence, encoding NeuroD1 into a host cell in vitro or in vivo.

A nucleic acid sequence encoding NeuroD1 is introduced into a host cell to produce NeuroD1 according to embodiments of the disclosure encodes SEQ ID NO: 2, SEQ ID NO: 4, or a variant thereof.

According to aspects of the present disclosure, the nucleic acid sequence identified herein as SEQ ID NO: 1 encodes SEQ ID NO: 2 and is included in an expression vector and expressed to produce NeuroD1. According to aspects of the present disclosure, the nucleic acid sequence identified herein as SEQ ID NO: 3 encodes SEQ ID NO: 4 and is included in an expression vector and expressed to produce NeuroD1.

It is appreciated that due to the degenerate nature of the genetic code, nucleic acid sequences substantially identical to SEQ ID NOs: 1 and 3 encode NeuroD1 and variants of NeuroD1, and that such alternate nucleic acids may be included in an expression vector and expressed to produce NeuroD1 and variants of NeuroD1. One of skill in the art will appreciate that a fragment of a nucleic acid encoding NeuroD1 protein can be used to produce a fragment of a NeuroD1 protein.

The term “expression vector” refers to a recombinant vehicle for introducing a nucleic acid encoding NeuroD1 into a host cell in vitro or in vivo where the nucleic acid is expressed to produce NeuroD1.

According to aspects of the present disclosure, an expression vector including SEQ ID NO: 1 or 3 or a substantially identical nucleic acid sequence encoding SEQ ID NO: 2 or SEQ ID NO: 4, or a variant thereof, is expressed to produce NeuroD1 in cells, in vitro or in vivo, containing the expression vector. The term “recombinant” is used to indicate a nucleic acid construct in which two or more nucleic acids are linked and which are not found linked in nature. Expression vectors include, but are not limited to plasmids, viruses, BACs and YACs. Particular viral expression vectors illustratively include those derived from adenovirus, adeno-associated virus, retrovirus, and lentivirus.

An expression vector contains a nucleic acid that includes segment encoding a polypeptide of interest operably linked to one or more regulatory elements that provide for transcription of the segment encoding the polypeptide of interest. The term “operably linked” as used herein refers to a nucleic acid in functional relationship with a second nucleic acid. The term “operably linked” encompasses functional connection of two or more nucleic acid molecules, such as a nucleic acid to be transcribed and a regulatory element. The term “regulatory element” as used herein refers to a nucleotide sequence which controls some aspect of the expression of an operably linked nucleic acid. Exemplary regulatory elements include an enhancer, such as, but not limited to: woodchuck hepatitis virus posttranscriptional regulatory element (WPRE); an internal ribosome entry site (IRES) or a 2A domain; an intron; an origin of replication; a polyadenylation signal (pA); a promoter; a transcription termination sequence; and an upstream regulatory domain, which contribute to the replication, transcription, post-transcriptional processing of an operably linked nucleic acid sequence. Those of ordinary skill in the art are capable of selecting and using these and other regulatory elements in an expression vector with no more than routine experimentation.

The term “promoter” as used herein refers to a DNA sequence operably linked to a nucleic acid sequence to be transcribed such as a nucleic acid sequence encoding NeuroD1. A promoter is generally positioned upstream of a nucleic acid sequence to be transcribed and provides a site for specific binding by RNA polymerase and other transcription factors. In specific embodiments, a promoter is generally positioned upstream of the nucleic acid sequence transcribed to produce the desired molecule, and provides a site for specific binding by RNA polymerase and other transcription factors.

As will be recognized by the skilled artisan, the 5′ non-coding region of a gene can be isolated and used in its entirety as a promoter to drive expression of an operably linked nucleic acid. Alternatively, a portion of the 5′ non-coding region can be isolated and used to drive expression of an operably linked nucleic acid. In general, about 500-6000 bp of the 5′ non-coding region of a gene is used to drive expression of the operably linked nucleic acid. Optionally, a portion of the 5′ non-coding region of a gene containing a minimal amount of the 5′ non-coding region needed to drive expression of the operably linked nucleic acid is used. Assays to determine the ability of a designated portion of the 5′ non-coding region of a gene to drive expression of the operably linked nucleic acid are well-known in the art.

Particular promoters used to drive expression of NeuroD1 according to methods of the present disclosure are “ubiquitous” or “constitutive” promoters, that drive expression in many, most, or all cell types into which the expression vector is transferred, in vitro or in vivo. Non-limiting examples of ubiquitous promoters that can be used in expression of NeuroD1 are cytomegalovirus promoter; simian virus 40 (SV40) early promoter; rous sarcoma virus promoter; adenovirus major late promoter; beta actin promoter; glyceraldehyde 3-phosphate dehydrogenase; glucose-regulated protein 78 promoter; glucose-regulated protein 94 promoter; heat shock protein 70 promoter; beta-kinesin promoter; ROSA promoter; ubiquitin B promoter; eukaryotic initiation factor 4A1 promoter and elongation Factor I promoter; all of which are well-known in the art and which can be isolated from primary sources using routine methodology or obtained from commercial sources. Promoters can be derived entirely from a single gene or can be chimeric, having portions derived from more than one gene.

Combinations of regulatory sequences may be included in an expression vector and used to drive expression of NeuroD1. A non-limiting example included in an expression vector to drive expression of NeuroD1 is the CAG promoter which combines the cytomegalovirus CMV early enhancer element, chicken beta-actin promoter, and the splice acceptor of the rabbit beta-globin gene.

Particular promoters used to drive expression of NeuroD1 according to methods described herein are those that drive expression preferentially in glial cells, particularly astrocytes and/or NG2 cells. Such promoters are termed “astrocyte-specific” and/or “NG2 cell-specific” promoters.

Non-limiting examples of astrocyte-specific promoters are glial fibrillary acidic protein (GFAP) promoter and aldehyde dehydrogenase 1 family, member L1 (Aldh1L1) promoter. Human GFAP promoter is shown herein as SEQ ID NO: 6. Mouse Aldh1L1 promoter is shown herein as SEQ ID NO: 7.

A non-limiting example of an NG2 cell-specific promoter is the promoter of the chondroitin sulfate proteoglycan 4 gene, also known as neuron-glial antigen 2 (NG2). Human NG2 promoter is shown herein as SEQ ID NO: 8.

Particular promoters used to drive expression of NeuroD1 according to methods described herein are those that drive expression preferentially in reactive glial cells. Non-limiting examples of reactive glial cells include reactive astrocytes and reactive NG2 cells. According to aspects of this disclosure, a reactive glial cell is a reactive astrocyte. According to aspects of the present disclosure, a reactive glial cell is a reactive NG2 cell. According to aspects of the present disclosure, promoters used to drive expression of NeuroD1 are termed “reactive astrocyte-specific” promoters. According to aspects of the present disclosure, promoters used to drive expression of NeuroD1 are termed “reactive NG2 cell-specific” promoters. A non-limiting example of a “reactive astrocyte-specific” promoter is the promoter of the lipocalin 2 (lcn2) gene. Mouse lcn2 promoter is shown herein as SEQ ID NO: 5.

Homologues and variants of ubiquitous and cell type-specific promoters may be used in expressing NeuroD1.

Promoter homologues and promoter variants can be included in an expression vector for expressing NeuroD1 according to the present disclosure. The terms “promoter homologue” and “promoter variant” refer to a promoter which has substantially similar functional properties to confer the desired type of expression, such as cell type-specific expression of NeuroD1 or ubiquitous expression of NeuroD1, on an operably linked nucleic acid encoding NeuroD1 compared to those disclosed herein. For example, a promoter homologue or variant has substantially similar functional properties to confer cell type-specific expression on an operably linked nucleic acid encoding NeuroD1 compared to GFAP, S100b, Aldh1L1, NG2, lcn2 and CAG promoters.

One of skill in the art will recognize that one or more nucleic acid mutations can be introduced without altering the functional properties of a given promoter. Mutations can be introduced using standard molecular biology techniques, such as site-directed mutagenesis and PCR-mediated mutagenesis, to produce promoter variants. As used herein, the term “promoter variant” refers to either an isolated naturally occurring or a recombinantly prepared variation of a reference promoter, such as, but not limited to, GFAP, S100b, Aldh1L1, NG2, lcn2 and pCAG promoters.

It is known in the art that promoters from other species are functional, e.g. the mouse Aldh1L1 promoter is functional in human cells. Homologues and homologous promoters from other species can be identified using bioinformatics tools known in the art, see for example, Xuan et al., 2005, Genome Biol 6:R72; Zhao et al., 2005, Nucl Acid Res 33:D103-107; and Halees et al. 2003, Nucl. Acids. Res. 2003 31: 3554-3559.

Structurally, homologues and variants of cell type-specific promoters of NeuroD1 and/or ubiquitous promoters have at least 80%, 81%, 82%, 83%, 84%, 85%, 86%, 87%, 88%, 89, 90%, 91%, 92%, 93%, 94%, 95%, 96%, 97%, 98%, 99%, or greater, nucleic acid sequence identity to the reference developmentally regulated and/or ubiquitous promoter and include a site for binding of RNA polymerase and, optionally, one or more binding sites for transcription factors.

A nucleic acid sequence which is substantially identical to SEQ ID NO: 1 or SEQ ID NO: 3 is characterized as having a complementary nucleic acid sequence capable of hybridizing to SEQ ID NO: 1 or SEQ ID NO: 3 under high stringency hybridization conditions.

In addition to one or more nucleic acids encoding NeuroD1, one or more nucleic acid sequences encoding additional proteins can be included in an expression vector. For example, such additional proteins include non-NeuroD1 proteins such as reporters, including, but not limited to, beta-galactosidase, green fluorescent protein and antibiotic resistance reporters.

According to aspects of the present disclosure, the recombinant expression vector encodes at least NeuroD1 of SEQ ID NO: 2, a protein having at least 95% identity to SEQ ID NO: 2, or a protein encoded by a nucleic acid sequence substantially identical to SEQ ID NO: 1.

According to aspects of the present disclosure, the recombinant expression vector encodes at least NeuroD1 of SEQ ID NO: 4, a protein having at least 95% identity to SEQ ID NO: 4, or a protein encoded by a nucleic acid sequence substantially identical to SEQ ID NO: 2.

Optionally, a reporter gene is included in a recombinant expression vector encoding NeuroD1. A reporter gene may be included to produce a peptide or protein that serves as a surrogate marker for expression of NeuroD1 from the recombinant expression vector. The term “reporter gene” as used herein refers to gene that is easily detectable when expressed, for example by chemiluminescence, fluorescence, colorimetric reactions, antibody binding, inducible markers and/or ligand binding assays. Exemplary reporter genes include, but are not limited to, green fluorescent protein (GFP), enhanced green fluorescent protein (EGFP), yellow fluorescent protein (YFP), enhanced yellow fluorescent protein (EYFP), cyan fluorescent protein (CFP), enhanced cyan fluorescent protein (ECFP), blue fluorescent protein (BFP), enhanced blue fluorescent protein (EBFP), red fluorescent protein (RFP), MmGFP (Zernicka-Goetz et al., Development, 124:1133-1137, 1997, dsRed, luciferase and beta-galactosidase (lacZ). mCherry is a monomeric red fluorescent protein derived from dsRed used as a reporter according to aspects of the present disclosure.

According to aspects of the present disclosure, SEQ ID NO: 9 is an example of a nucleic acid comprising a CAG promoter operably linked to a nucleic acid encoding NeuroD1, a nucleic acid sequence encoding enhanced green fluorescent protein (EGFP), an enhancer, the woodchuck hepatitis post-transcriptional regulatory element (WPRE) and a. IRES separating the nucleic acid encoding NeuroD1 and the nucleic acid encoding EGFP.

According to aspects of the present disclosure, SEQ ID NO: 9 is inserted into an expression vector for expression of NeuroD1 and the reporter gene EGFP.

Optionally, according to aspects of the present disclosure, the IRES and nucleic acid encoding EGFP are removed from SEQ ID NO: 9 and the remaining nucleic acid sequence including CAG promoter and operably linked nucleic acid encoding NeuroD1 is inserted into an expression vector for expression of NeuroD1. The WPRE or another enhancer is optionally included.

The process of introducing genetic material into a recipient host cell, such as for transient or stable expression of a desired protein encoded by the genetic material in the host cell is referred to as “transfection,” or “transduction.” Transfection techniques are well-known in the art and include, but are not limited to, electroporation, particle accelerated transformation also known as “gene gun” technology, liposome-mediated transfection, calcium phosphate or calcium chloride co-precipitation-mediated transfection, DEAE-dextran-mediated transfection, microinjection, polyethylene glycol mediated transfection, and heat shock mediated transfection. Transduction refers to virus-mediated introduction of genetic material into a recipient host cell.

Virus-mediated transduction may be accomplished using a viral vector such as those derived from adenovirus, AAV and lentivirus.

Optionally, a host cell is transfected or transduced ex-vivo and then re-introduced into a host organism. For example, cells or tissues may be removed from a subject, transfected or transduced with an expression vector encoding NeuroD1 and then returned to the subject.

Introduction of a recombinant expression vector including a nucleic acid encoding NeuroD1, or a functional fragment thereof, into a host glial cell in vitro or in vivo for expression of exogenous NeuroD1 in the host glial cell to convert the glial cell to a functional neuron is accomplished by any of various transfection or transduction methodologies.

Expression of exogenous NeuroD1 in the host glial cell to convert the glial cell to a functional neuron is achieved by introduction of mRNA encoding NeuroD1, or a functional fragment thereof, to the host glial cell in vitro or in vivo according to aspects of the present disclosure.

Expression of exogenous NeuroD1 in the host glial cell to convert the glial cell to a functional neuron is achieved by introduction of DNA encoding NeuroD1, or a functional fragment thereof, to the host glial cell in vitro or in vivo according to aspects of the present disclosure.

Expression of exogenous NeuroD1 in the host glial cell to convert the glial cell to a functional neuron is achieved by introduction of NeuroD1 protein to the host glial cell in vitro or in vivo according to aspects of the present disclosure.

Details of these and other techniques are known in the art, for example, as described in J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; and Engelke, D. R., RNA Interference (RNAi): Nuts and Bolts of RNAi Technology, DNA Press LLC, Eagleville, P A, 2003.

Expression of NeuroD1 using a recombinant expression vector is accomplished by introduction of the expression vector into a eukaryotic or prokaryotic host cell expression system such as an insect cell, mammalian cell, yeast cell, bacterial cell or any other single or multicellular organism recognized in the art. Host cells are optionally primary cells or immortalized derivative cells. Immortalized cells are those which can be maintained in-vitro for at least 5 replication passages.

Host cells containing the recombinant expression vector are maintained under conditions wherein NeuroD1 is produced. Host cells may be cultured and maintained using known cell culture techniques such as described in Celis, Julio, ed., 1994, Cell Biology Laboratory Handbook, Academic Press, N.Y. Various culturing conditions for these cells, including media formulations with regard to specific nutrients, oxygen, tension, carbon dioxide and reduced serum levels, can be selected and optimized by one of skill in the art.

According to aspects of the present disclosure, a recombinant expression vector including a nucleic acid encoding NeuroD1 is introduced into glial cells of a subject. Expression of exogenous NeuroD1 in the glial cells “converts” the glial cells into functional neurons.

The terms “converts” and “converted” are used herein to describe the effect of expression of NeuroD1, a variant thereof, or a functional fragment thereof, in a glial cell resulting in a change of a glial cell, and in particular cases an astrocyte, or reactive astrocyte phenotype to a functional neuronal phenotype. Similarly, the phrases “NeuroD1 converted neurons” and “converted neurons” are used herein to designate a cell including exogenous NeuroD1 protein or a functional fragment thereof which has consequent functional neuronal phenotype.

The term “phenotype” refers to well-known detectable characteristics of the cells referred to herein. The functional neuronal phenotype can be, but is not limited to, one or more of: neuronal morphology, expression of one or more neuronal markers, electrophysiological characteristics of neurons, synapse formation and release of neurotransmitter. For example, neuronal phenotype encompasses but is not limited to: characteristic morphological aspects of a neuron such as presence of dendrites, an axon and dendritic spines; characteristic neuronal protein expression and distribution, such as presence of synaptic proteins in synaptic puncta, presence of MAP2 in dendrites, presence of one or more of: neuronal nuclear protein (NeuN), GABA, glutamate decarboxylase (GAD) such as GAD67, Forkhead-box-G1 (FoxG1), T-brain-1 (Tbr1), Cux1, Ctip2, parvalbumin (PV), calretinin (CR), neuropeptide Y (NPY), and somatostatin (SST); and characteristic electrophysiological signs such as spontaneous and evoked synaptic events.

In a further example, glial phenotype such as astrocyte phenotype and reactive astrocyte phenotypes encompasses but is not limited to: characteristic morphological aspects of astrocytes and reactive astrocytes such as a generally “star-shaped” morphology; and characteristic astrocyte and reactive astrocyte protein expression, such as presence of glial fibrillary acidic protein (GFAP).

According to aspects of the present disclosure, a recombinant expression vector including a nucleic acid encoding NeuroD1, a variant thereof, or a functional fragment thereof, is introduced into astrocytes of a subject. Expression of exogenous NeuroD1, a variant thereof, or a functional fragment thereof, in the astrocytes cells “converts” the astrocytes into functional neurons.

According to aspects of the present disclosure, a recombinant expression vector including a nucleic acid encoding NeuroD1, a variant thereof, or a functional fragment thereof, thereof is introduced into reactive astrocytes of a subject. Expression of exogenous NeuroD1, a variant thereof, or a functional fragment thereof, in the reactive astrocytes “converts” the reactive astrocytes into functional neurons.

According to aspects of the present disclosure, a recombinant expression vector including a nucleic acid encoding NeuroD1, a variant thereof, or a functional fragment thereof, is introduced into NG2 cells of a subject. Expression of exogenous NeuroD1, a variant thereof, or a functional fragment thereof, in the NG2 cells “converts” the NG2 cells into functional neurons.

An expression vector including a nucleic acid encoding NeuroD1, a variant thereof, or a functional fragment thereof, DNA encoding NeuroD1, a variant thereof, or a functional fragment thereof, mRNA encoding NeuroD1, a variant thereof, or a functional fragment thereof, and/or NeuroD1 protein, a variant thereof, full-length or a functional fragment thereof, is optionally associated with a carrier for introduction into a host cell in vitro or in vivo.

In particular aspects, the carrier is a particulate carrier such as lipid particles including liposomes, micelles, unilamellar or mulitlamellar vesicles; polymer particles such as hydrogel particles, polyglycolic acid particles or polylactic acid particles; inorganic particles such as calcium phosphate particles such as described in for example U.S. Pat. No. 5,648,097; and inorganic/organic particulate carriers such as described for example in U.S. Pat. No. 6,630,486.

A particulate carrier can be selected from among a lipid particle; a polymer particle; an inorganic particle; an organic particle; and a hybrid inorganic/organic particle. A mixture of particle types can also be included as a particulate pharmaceutically acceptable carrier.

A particulate carrier is typically formulated such that particles have an average particle size in the range of about 1 nm-10 microns. In particular aspects, a particulate carrier is formulated such that particles have an average particle size in the range of about 1 nm-100 nm.

Further description of liposomes and methods relating to their preparation and use may be found in Liposomes: A Practical Approach (The Practical Approach Series, 264), V. P. Torchilin and V. Weissig (Eds.), Oxford University Press; 2nd ed., 2003. Further aspects of nanoparticles are described in S. M. Moghimi et al., FASEB J. 2005, 19, 311-30.

Detection of expression of exogenous NeuroD1 following introduction of a recombinant expression vector including a nucleic acid encoding the exogenous NeuroD1 or a functional fragment thereof is accomplished using any of various standard methodologies including, but not limited to, immunoassays to detect NeuroD1, nucleic acid assays to detect NeuroD1 nucleic acids and detection of a reporter gene co-expressed with the exogenous NeuroD1.

The term “nucleic acid” refers to RNA or DNA molecules having more than one nucleotide in any form including single-stranded, double-stranded, oligonucleotide or polynucleotide. The term “nucleotide sequence” refers to the ordering of nucleotides in an oligonucleotide or polynucleotide in a single-stranded form of nucleic acid.

The term “NeuroD1 nucleic acid” refers to an isolated NeuroD1 nucleic acid molecule and encompasses isolated NeuroD1 nucleic acids having a sequence that has at least 70%, 75%, 80%, 85%, 90%, 91%, 92%, 93%, 94%, 95%, 96%, 97%, 98%, 99%, or greater, identity to the DNA sequence set forth in SEQ ID NO: 1 or SEQ ID NO: 3, or the complement thereof, or a fragment thereof, or an isolated nucleic acid, such as RNA or DNA, molecule having a sequence that hybridizes under high stringency hybridization conditions to the nucleic acid set forth as SEQ ID NO: 1 or SEQ ID NO: 3, a complement thereof, or a fragment thereof. The term “isolated” with reference to a NeuroD1 nucleic acid molecule indicates that the molecule is not in the genome of an organism from which it originated under control of the NeuroD1 promoter in that location.

The nucleic acid of SEQ ID NO: 3 is an example of an isolated DNA molecule having a sequence that hybridizes under high stringency hybridization conditions to the nucleic acid set forth in SEQ ID NO: 1.

A fragment of a NeuroD1 nucleic acid is any fragment of a NeuroD1 nucleic acid that is operable in aspects of the present disclosure including a NeuroD1 nucleic acid.

A nucleic acid probe or primer able to hybridize to a target NeuroD1 RNA or DNA molecule, such as mRNA or cDNA, can be used for detecting and/or quantifying the RNA or DNA, such as mRNA or cDNA, encoding NeuroD1 protein. A nucleic acid probe can be an oligonucleotide of at least 10, 15, 30, 50 or 100 nucleotides in length and sufficient to specifically hybridize under stringent conditions to NeuroD1 RNA or DNA, such as mRNA or cDNA, or a complementary sequence thereof. A nucleic acid primer can be an oligonucleotide of at least 10, 15 or 20 nucleotides in length and sufficient to specifically hybridize under stringent conditions to the RNA or DNA, such as mRNA or cDNA, or complementary sequence thereof.

The terms “complement” and “complementary” refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′. Further, the nucleotide sequence 3′-TCGA- is 100% complementary to a region of the nucleotide sequence 5′-TTAGCTGG-3′.

The terms “hybridization” and “hybridizes” refer to pairing and binding of complementary nucleic acids. Hybridization occurs to varying extents between two nucleic acids depending on factors such as the degree of complementarity of the nucleic acids, the melting temperature, Tm, of the nucleic acids and the stringency of hybridization conditions, as is well known in the art. The term “stringency of hybridization conditions” refers to conditions of temperature, ionic strength, and composition of a hybridization medium with respect to particular common additives such as formamide and Denhardt's solution.

Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002. High stringency hybridization conditions are those which only allow hybridization of substantially complementary nucleic acids. Typically, nucleic acids having about 85-100% complementarity are considered highly complementary and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize.

The terms “specific hybridization” and “specifically hybridizes” refer to hybridization of a particular nucleic acid to a target nucleic acid without substantial hybridization to nucleic acids other than the target nucleic acid in a sample.

Stringency of hybridization and washing conditions depends on several factors, including the Tm of the probe and target and ionic strength of the hybridization and wash conditions, as is well-known to the skilled artisan. Hybridization and conditions to achieve a desired hybridization stringency are described, for example, in Sambrook et al., Molecular Cloning: A Laboratory Manual, Cold Spring Harbor Laboratory Press, 2001; and Ausubel, F. et al., (Eds.), Short Protocols in Molecular Biology, Wiley, 2002.

An example of high stringency hybridization conditions is hybridization of nucleic acids over about 100 nucleotides in length in a solution containing 6×SSC, 5×Denhardt's solution, 30% formamide, and 100 micrograms/ml denatured salmon sperm at 37° C. overnight followed by washing in a solution of 0.1×SSC and 0.1% SDS at 60° C. for 15 minutes. SSC is 0.15M NaCl/0.015M Na citrate. Denhardt's solution is 0.02% bovine serum albumin/0.02% FICOLL/0.02% polyvinylpyrrolidone. Under highly stringent conditions, SEQ ID NO: 1 and SEQ ID NO: 3 will hybridize to the complement of substantially identical targets and not to unrelated sequences.

Embodiments of inventive compositions and methods are illustrated in the following examples. These examples are provided for illustrative purposes and are not considered limitations on the scope of inventive compositions and methods.

### Example

Material and Methods

Mouse Model of Closed Head Injury

Wild type (WT) C57BL/6J and FVB/N-Tg(GFAP::GFP) 14Mes/J transgenic mice were purchased from Jackson Laboratory. Mice were housed in a 12 hr light/dark cycle and supplied with sufficient food and water. Adult mice (25-35 g) of both genders, aged 4-6 months old, were used in this example.

Mice were anesthetized with ketamine/xylazine (120 mg/kg ketamine; 8 mg/kg xylazine) by intraperitoneal (IP) injection. After being fully anesthetized, each mouse was transferred onto a stereotaxic apparatus and the head fixed on shape-matched foam. The foam was made by soft plastic materials to absorb superfluous impact as well as to stabilize the animal's head. An incision was made along the midline to fully expose the impact site on the skull, which is above mouse motor cortex with coordinates of 1.0 mm anterior to the Bregma and 1.5 mm lateral to the midline. The ear bars were removed to avoid injury to the ear canals before impact induction. The electro-magnetic controlled device, Impactor One, was purchased from Leica Biosystems® for TBI induction. Impact force of larger than 5.0 m/s was avoided to prevent skull fracture and death.

An impactor tip of 2 mm diameter was used at velocity of 5 m/s, dwelling time of 200 ms, and impact depth of 1.0 mm, to cause focal closed head injury. The bottom plane of impactor tip was adjusted to be tangential to the impact site on the skull, in order to deliver the impact force evenly to the skull and reduce the risk of skull fracture. Animals with an obvious skull fracture are excluded from the experiments and euthanized immediately.

After the impact delivery and incision suture, the animal was removed from the stereotaxic apparatus, placed on a heating pad and posttraumatic oxygen was immediately administered at a rate of 3-5 liter 02 per minute until deep and regular breathing was restored. Animals were kept on a heated pad and observed until they recovered from the procedure, and then monitored daily for a minimum of 7 days post-surgery. In the first 3 days following the impact, buprenorphine (0.05 mg/kg) was given two times daily to alleviate pain.

Virus Injection

7 days after closed head injury, mice were randomly selected for injection administration of either a virus encoding NeuroD1 or a control virus. Mice were anesthetized with ketamine/xylazine (120 mg/kg ketamine; 8 mg/kg xylazine) by intraperitoneal injection and placed in a stereotaxic apparatus. An incisor bar with a nose bridge holder and two ear bars were used to fix the head. After a midline incision was made, a small hole of ˜1 mm was drilled in the skull at the center of impact site (coordinates: 1.0 mm anterior, 1.5 mm left lateral to Bregma). The selected virus, 1.5 μL (AAV9)hGFAP::GFP or (AAV9)hGFAP::NeuroD1-GFP, or 3 μL retrovirus carrying NeuroD1-GFP or GFP control, was injected into the injured brain region using a motorized micro pump injector at a speed of 0.15 μL/min for 10 min with a 5 μL Hamilton brand glass syringe with a 33 Gauge needle. After injection, the needle was maintained in place for an additional 3 minutes before being fully withdrawn. Post-surgery, mice recovered on heating pad until free movement was observed. Mice were singly housed and carefully monitored daily for at least one week or until sacrifice.

Neural Projection Tracing by Virus or Dye

For anterograde tracing, adeno-associated virus (AAV) with hSyn::Cre plus CAG::FLEX-mCherry-P2A-mCherry was injected into thalamus (coordinates: 2.0 mm posterior, 1.1 mm left lateral to Bregma; 2.8 mm ventral to skull surface). For retrograde tracing, cholera toxin B subunit fused with 647 fluorescent probe (CTB-647) was injected in the cortex contralateral to injury site (coordinates: 1.0 mm anterior, 1.5 mm right lateral to Bregma, 1.6 mm ventral to skull surface). Animals were sacrificed 7 days later and brain samples collected for analysis.

AAV Vector Construction

The plasmid pAAV-GFAP-hChR2(H134R)-mCherry was obtained from Addgene (plasmid #27055; RRID:Addgene_27055). To construct pAAV-hGFAP::GFP and pAAV-hGFAP::NeuroD1-P2A-GFP vectors cDNAs coding GFP or NeuroD1 were produced by PCR using the retroviral constructs as described in detail in Guo et al., Cell Stem Cell 14, 188-202, 2014. The GFP gene or NeuroD1 fused with P2A-GFP gene was subcloned into the pAAV-GFAP-hChR2(H134R)-mCherry vector with hChR2(H134R)-mCherry cut out between KpnI and BsrGI sites. For the plasmid of pAAV-Synapsin::Cre, the Cre gene was obtained by PCR from hGFAP-Cre (Addgene plasmid #40591) and inserted into AAV phSyn(S)-FlpO-bGHpA (Addgene plasmid #51669) between KpnI and BmtI sites with FlpO replaced to generate pAAV-hSyn:Cre vector. The pAAV-FLEX-mCherry-P2A-mCherry vector was constructed as described in detail in Chen et al., Mol Ther., 2020 Jan. 8; 28(1):217-234. Plasmid constructs were sequenced for verification.

AAV Virus Production

Recombinant AAV9 was produced in 293AAV cells (Cell Biolabs, San Diego, Calif., USA). Polyethylenimine (PEI, linear, MW 25,000) was used for transfection of triple plasmids: the pAAV expression vector, pAAV9-RC (Cell Biolabs, San Diego, Calif., USA) and pHelper (Cell Biolabs, San Diego, Calif., USA). 72 hours post-transfection, cells were scraped in their medium and centrifuged, frozen, and thawed four times by placing them alternately in dry ice or ethanol and a 37° C. water bath. AAV crude lysate was purified by centrifugation at 54,000 rpm for 1 hour in discontinuous iodixanol gradients with a Beckman SW55Ti rotor. The virus-containing layer was extracted, and viruses were concentrated by Millipore Amicon Ultra Centrifugal Filters. Virus titers were 2.2×10¹¹ genome copies per milliliter (GC/mL) for hGFAP::GFP, 2.3×10¹¹ GC/mL for hGFAP::ND1-GFP, 4.6×10¹¹ GC/mL for hSyn::Cre, and 1.6×10¹² GC/mL for CAG::FLEX-mCherry-P2A-mCherry, determined by QuickTiter AAV Quantitation Kit (Cell Biolabs, San Diego, Calif., USA).

Retrovirus Production

The pCAG-NeuroD1-IRES-GFP and pCAG-GFP were constructed as previously described (Guo et al., Cell Stem Cell, 14:188-202 (2014)). To package retroviral particles, gpg helper-free HEK cells were transfected with the target plasmid together with vesicular stomatitis virus G protein (VSV-G) vector to produce the retroviruses expressing NeuroD1 or GFP. The titer of retroviral particles was about 10⁷ particles/mL, determined after transduction of HEK cells.

Immunohistochemistry

Mouse brains were collected as described in detail in Guo et al., Cell Stem Cell 14, 188-202, 2014). Briefly, animals were injected with 2.5% Avertin for anesthesia. Transcardial perfusion with artificial cerebral spinal fluid (ACSF) was performed to systemically wash out the blood. Then, brains were dissected out and post-fixed in 4% paraformaldehyde (PFA) at 4° C. overnight. After fixation, brain tissues were sectioned into 40 μm sections using a Leica-1000 vibratome. Brain slices were washed 3 times with phosphate-buffered saline (PBS) followed by permeablization in 2% Triton X-100 in PBS for 10 minutes. Then brain sections were blocked in 5% normal donkey serum and 0.3% Triton X-100 in PBS for 2 hours. The primary antibodies were added into blocking buffer and incubated with brain sections for overnight at 4° C. Primary antibodies were rinsed off with PBS 3 times followed by secondary antibody incubation for 2 hours at room temperature (RT). After being washed with PBS, brain sections were mounted onto a glass slide with an anti-fading mounting solution (Invitrogen). Images were acquired with confocal microscopes (Olympus FV1000 or Zeiss LSM800). To ensure antibody specificity, only secondary antibody was used for immunostaining as a side-by-side control, with no distinct signal detected.

Wheel Running and c-Fos Detection

28 days after NeuroD1 virus injection following closed head injury (CHI), the animals were placed in a running wheel. Thirty minutes after actively running, the mice were placed back into the home cage. One hour later they were sacrificed and perfused for c-Fos immunostaining.

Electrophysiology

Brain slice recording were performed as described in detail in Guo et al., Cell Stem Cell 14, 188-202, 2014; Wu et al., Nat Commun 5, 4159, 2014). At 7, 14, 28, and 56 days after virus injection, the mice were anaesthetized with 2.5% avertin, and then perfused with NMDG-based cutting solution containing (in mM): 93 NMDG, 93 HCl, 2.5 KCl, 1.25 NaH2PO₄, 30 NaHCO₃, 20 HEPES, 15 glucose, 12 N-Acetyl-L-cysteine, 5 sodium ascorbate, 2 thiourea, 3 sodium pyruvate, 7 MgSO₄, and 0.5 CaCl₂, at pH 7.3-7.4, at 300 mOsm, and bubbled with 95% O₂/5% CO₂. Coronal sections of 300 μm thickness were cut around AAV-injected cortical areas with a vibratome (VT1200S, Leica, Germany) at room temperature. Slices were collected and incubated at 33.0±1.0° C. in oxygenated NMDG cutting solution for 10-15 minutes. Then, slices were transferred to holding solutions with continuous 95% O₂/5% CO₂ bubbling and containing (in mM): 92 NaCl, 2.5 KCl, 1.25 NaH2PO₄, 30 NaHCO₃, 20 HEPES, 15 glucose, 12 N-Acetyl-L-cysteine, 5 sodium ascorbate, 2 thiourea, 3 sodium pyruvate, 2 MgSO₄, and 2 CaCl₂. After recovery for at least 0.5 hour at room temperature in the holding solution, a single slice was transferred to the recording chamber continuously perfused with standard aCSF (artificial cerebral spinal fluid) saturated by 95% O₂/5% CO₂ at 33.0±1.0° C. The standard aCSF contained (in mM): 124 NaCl, 2.5 KCl, 1.25 NaH₂PO₄, 26 NaHCO₃, 10 glucose, 1.3 MgSO₄, and 2.5 CaCl₂. To detect action potential firing in NeuroD1-GFP-infected neurons, whole-cell recordings were performed with pipette solution containing (in mM): 135 K-Gluconate, 10 KCl, 5 Na-phosphocreatine, 10 HEPES, 2 EGTA, 4 MgATP and 0.3 Na₂GTP, pH 7.3 adjusted with KOH, 280-290 mOsm. Depolarizing currents were injected to elicit action potentials under current-clamp model. To record spontaneous excitatory postsynaptic currents (sEPSCs) and spontaneous inhibitory postsynaptic currents (sIPSCs), pipette solution contained (in mM): 120 Cs-methanesulfonate, 10 KCl, 10 Na-phosphocreatine, 10 HEPES, 5 QX-314, 1 EGTA, 4 MgATP and 0.3 Na₂GTP, pH 7.3 adjusted with KOH, 280-290 mOsm. To labeled recorded neurons, 0.5% biocytin (Sigma, Cat. B4261) was added to the pipette solution. The cell membrane potentials were held at −70 mV (the reversal potential of GABAA receptors) for sEPSC recording, and 0 mV (the reversal potential of ionotropic glutamate receptors) for sIPSC recording, respectively. Data were collected with a MultiClamp 700A amplifier and analyzed with pClamp 9.0 and Clampfit 10.6 software (Molecular Devices).

Confocal Imaging and Analysis

Injury Area Definition.

The cortical areas around injury site from 750 μm to 2250 μm lateral to the midline were defined as total injury area for analysis. The superficial layer with width less than 600 μm and depth less than 450 μm from the impact center was defined as injury core. The middle layer with width 600-1000 μm and depth 450-900 μm from the impact center was defined as peri-injury area.

Cell Density Analysis.

The sections of mice brains were imaged by the Z-stack and tile function of Olympus FV-1000 with 40× oil lens after immunostaining. The range of Z-stack was set to be 5 layers with 1.5 μm step size around the center plane of the mounted slice. In each section, 3 squares of Z-stack images (resolution: 512×512, 0.621 μm/pixel) were selected inside the injury core or the peri-injury area for quantification.

Cell Conversion and Subtype Ratio Analysis.

Three sections were selected which were inside the injury and infection range. One section was close to the center of injury and infection. The other two sections were at the middle position of anterior half or posterior half of infection area relatively. Single layer confocal images of each brain section were taken for quantification by Olympus FV-1000 with 40× oil lens.

Data Analysis and Statistics

Prism 6 Graphpad software was used for statistical analysis and bar graphs. For comparison of two data sets, Student's t-test was conducted. For comparison of 3 data sets, one-way or two-way analysis of variance (ANOVA) was performed, followed by post-hoc tests. Statistical significance was set as p<0.05. Data were presented as mean±SEM.

Results

Establishment of a Focal Closed Head Injury Model and the Pathology of Neurons and Glia Cells in the Injury Site.

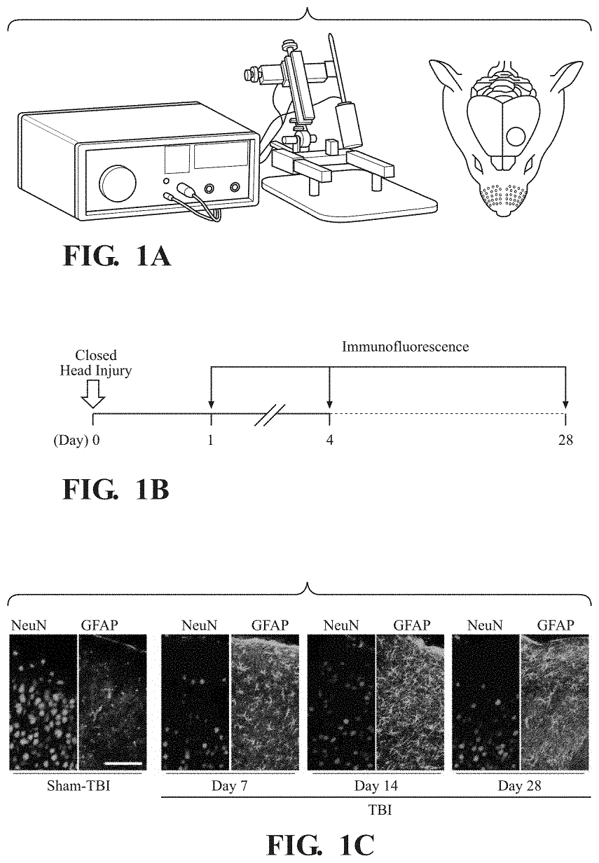

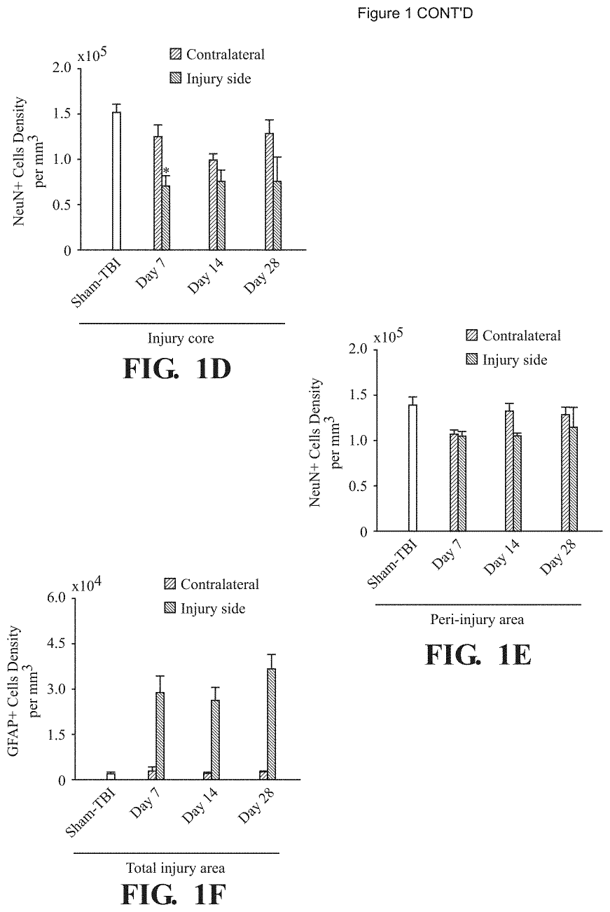

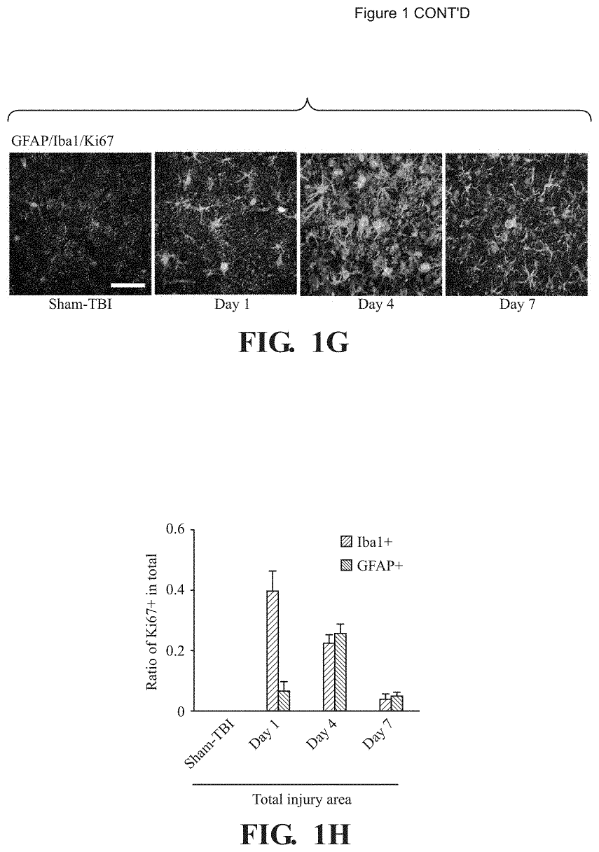

In this example, an electromagnetic controlled device, Leica impactor one, shown diagrammatically in FIG. 1A left, was used to induce a precisely controlled CHI, a type of TBI, to exposed skull above mouse motor cortex, see FIG. 1A right.

After CHI, the pathological outcomes were investigated at different time points, see FIG. 1B. Investigation of pathology was focused on astrocytes and neurons. The preliminary experiments had proved the primary and secondary brain injuries after CHI in our model were mainly located focally under the impact site.

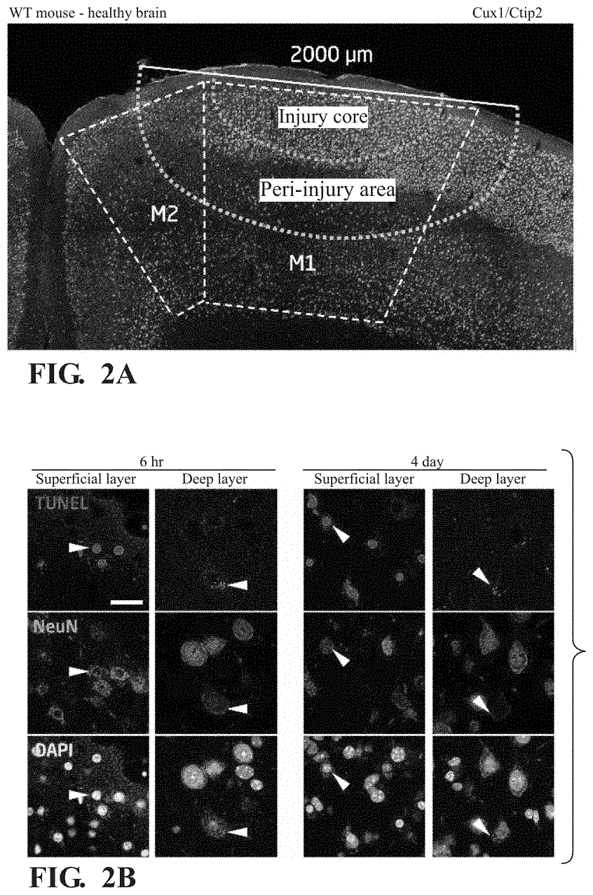

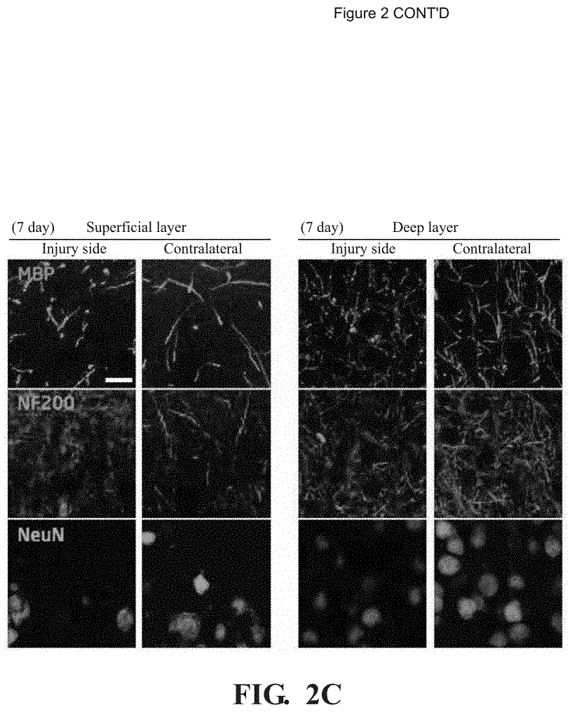

FIGS. 2A, 2B, and 2C demonstrate neuronal death and degeneration at injury site after CHI.

As shown in FIG. 2A, 3 brain regions close to the impact center were considered as the injury core, located mostly inside the smallest semi-circular area defined by a dashed line. 5 regions next to the injury core were taken as peri-injury area, located mostly inside the larger semi-circular area defined by a dashed line. All the regions inside the dashed rectangular boxes defined by dashed lines were taken as region of interest (ROI) for analysis.

Compared to the Sham-TBI group, the NeuN signal obviously decreased, and the GFAP signal increased greatly, at 7 days as well as 14 and 28 days after injury, see FIG. 1C. The density of NeuN+ or GFAP+ cells in the injury site, the contralateral side, and the Sham-TBI contro were quantified. The results indicated that both the injury side and the contralateral side had fewer NeuN+ cells, see FIG. 1D and FIG. 1E, and more GFAP+ cells, see FIG. 1F, than the brain from Sham-TBI group. The injury core underwent the most severe NeuN loss, see FIG. 1D, which indicated that there was neuronal death and astrocytic reactivation after CHI.

To confirm the neuronal death around the injury core after CHI, the brain sample at early time points after CHI, such as 6 hours and 4 days after injury, were collected and assayed to detect a biomarker of cell apoptosis—Terminal deoxynucleotidyl transferase dUTP nick end labeling TUNEL. The results showed that many neurons in the superficial layer close to the injury core had strong TUNEL and chaotic NeuN signal, which indicated they were dying cells. Further, even in the deep layer beneath the injury area, some neurons showed accumulated TUNEL signal and weak NeuN signal compared to other neighboring neurons, see FIG. 2B. Based on their morphology, these cells could be the pyramidal neurons in the deep cortical layer. Thus, the CHI, with the primary injury by mechanical force or the following secondary injury damaged the cells and induced apoptosis. In other brain areas, like the regions in the same hemisphere far away from the impact site or the contralateral side, no TUNEL signal was detected.

To further investigate the effect of CHI on neuronal processes, brain samples from animals 1 week post-CHI were immunostained to detect myelin basic protein (MBP) and high (200 kD) molecular weight neurofilament proteins (NF200). Both markers represent the morphology of neuronal processes and reflect the health status of the cortex. By comparing the injury side with the contralateral side, many enlarged axon terminals with strong MBP signal were found on the injury side, see FIG. 2C, top images) which may represent injured axons forming “retraction bulbs”. The NF200 staining also indicated CHI caused cytoskeletal breakdown around the impact site see FIG. 2C, middle images. This is consistent with results in other TBI models.

Accompanying the neuronal degeneration, the astrocytes around the injury core became greatly reactive compared to the non-injury side or the sham group, see FIG. 1C and FIG. 1F. Further, by staining the cell proliferation marker, Ki67, at different time points after CHI, it was found that the proliferation rate of astrocytes reached a peak at 4 days post-injury and go quiescent after 7 days post-injury, see FIG. 1G and FIG. 1H. The microglia population, marked by Iba1 staining, see FIG. 1G, appears to have a proliferation curve peaking earlier, at 1 day post-injury, see FIG. 1H.

Astrocyte-to-Neuron Conversion In Situ by NeuroD1 after Closed Head Injury in Mouse Neocortex.

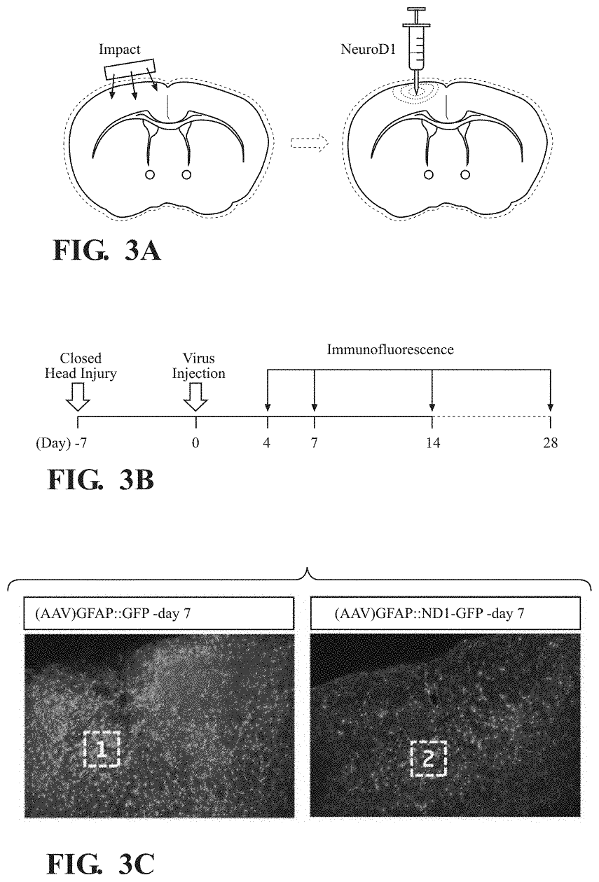

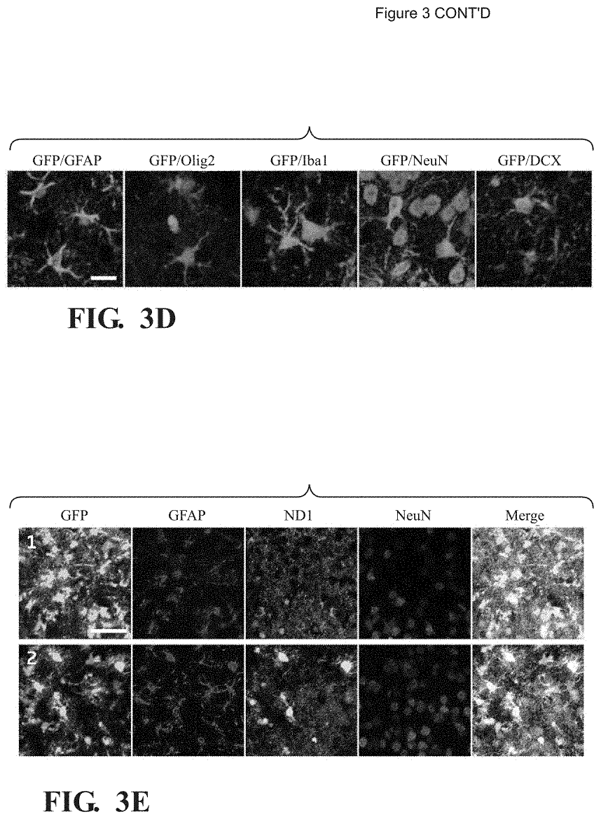

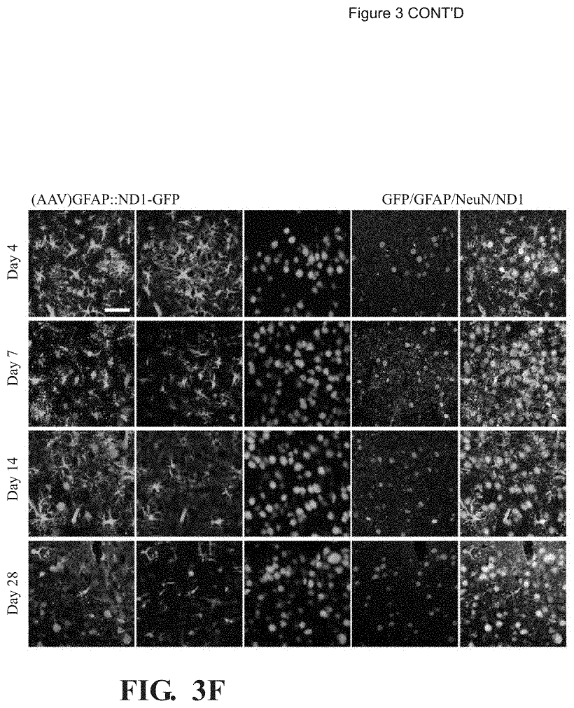

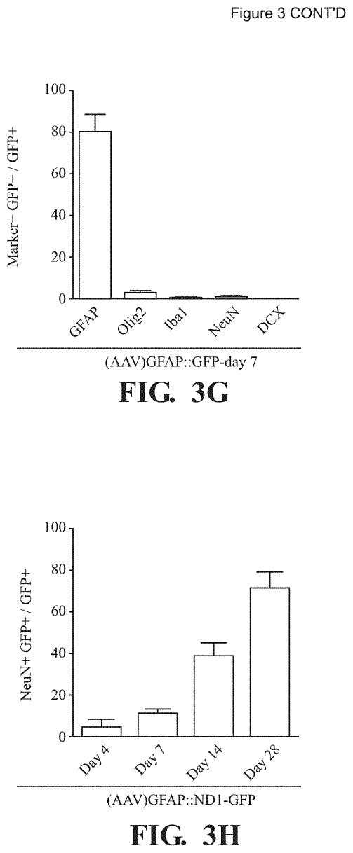

To provide exogenous NeuroD1 to mouse cortical astrocytes, an AAV vector, recombinant serotype AAV9, was constructed to express NeuroD1 in mouse cortical astrocytes under the direct control of a human GFAP promotor with enhanced green fluorescence protein (GFP) as indicator of expression, the construct designated hGFAP::NeuroD1-P2A-GFP (also called (AAV)GFAP::ND1-GFP). FIGS. 3A, 3B, 3C, 3D, 3E, 3F, 3G, and 3H show astrocyte-to-neuron conversion in situ by NeuroD1 (ND1) after closed head injury in mouse neocortex.

As shown diagrammatically in FIG. 3A and FIG. 3B, an AAV expression vector expressing NeuroD1 (AAV)GFAP::ND1-GFP or a control expression vector (AAV)GFAP::GFP) was injected into the injury site 7 days after CHI. Following administration of these vectors, cells were examined to determine which were infected by each virus and which express the encoded gene under control of the GFAP promotor. FIG. 3C is a set of representative images showing the injured cortex 7 days after injection of AAV-GFAP::GFP virus (control group, left panel) or injection of AAV-GFAP::ND1-GFP virus (ND1 group, right panel).

By comparison of GFP fluorescence with immunostaining for different cell subtype markers in the brain samples of control group 7 days after virus injection, the percentages of labeled astrocytes (GFAP), oligodendrocytes (olig2), microglia cells (Iba1), neurons (NeuN), as well as stem cells (DCX) was determined, see FIG. 3D. The results indicated that most of the GFP-expressing cells were astrocytes, some were oligodendrocytes, few were neurons, very few were microglia cells and almost none were stem cells, see FIG. 3G. This confirmed that the converted neurons in the ND1 group originated from astrocytes. At this time point, most GFP+ cells in ND1 group were still glia cells with GFAP, see FIG. 3E-2. However, NeuroD1 staining indicated that these cells had high expression of NeuroD1 inside, see FIG. 3E, which was the fundamental difference from the control.

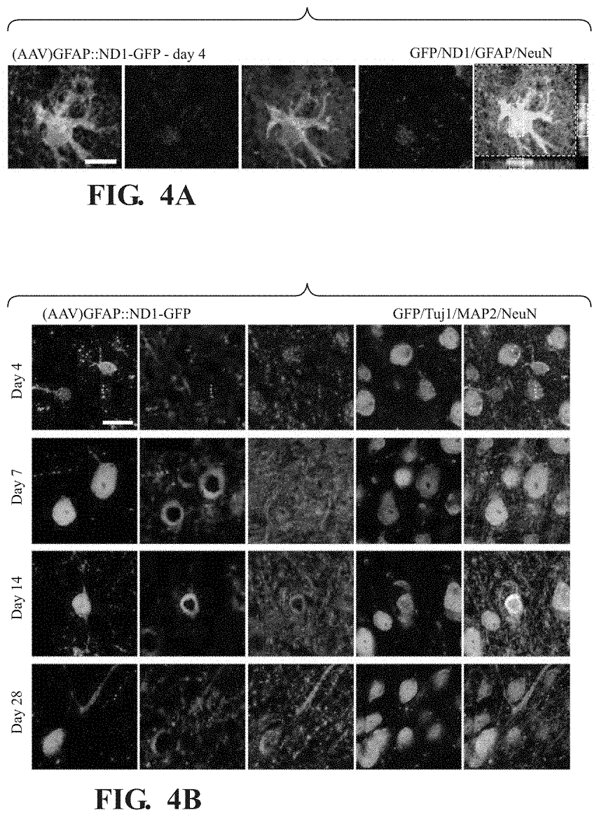

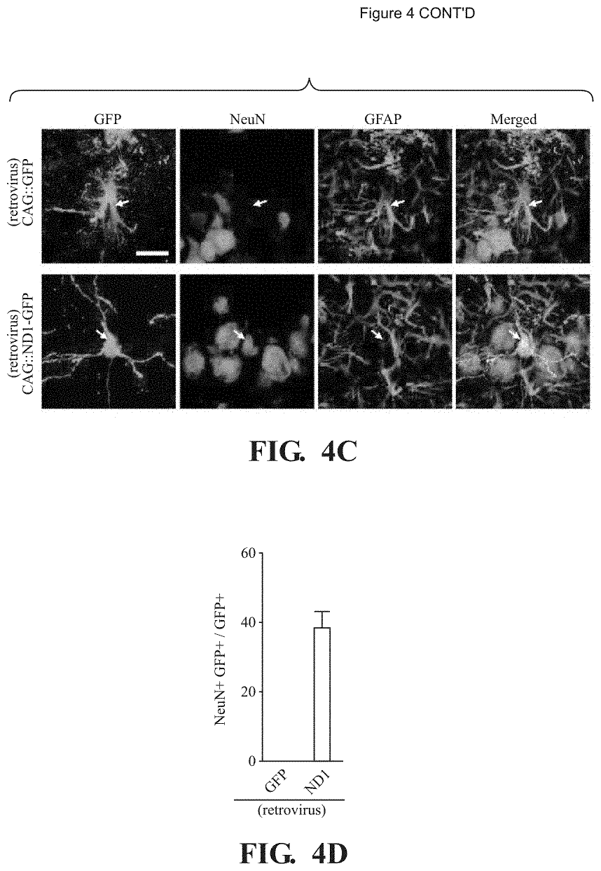

Brain samples from animals in the “ND1 group” (i.e. those injected with (AAV)GFAP::ND1-GFP) were analyzed at multiple time points, such as 4/7/14/28 days after virus injection, to show the process of astrocyte-to-neuron conversion. At very early time points, such as 4 days post-injection (dpi), almost all the GFP+ cells were GFAP+ and NeuN− without visible NeuroD1 (ND1) expression by immunostaining, see FIG. 3F-Day 4, i.e. 4 dpi). However, some astrocytes had started to transformed at this time point. About 5% GFP+ cells had NeuN signal. Some of them even still had GFAP signal at the same time, which was called transitional stage, see FIG. 4A. FIGS. 4A, 4B, 4C, and 4D show a transitional stage of astrocyte-to-neuron conversion, maturation of converted neurons, and conversion induced by retrovirus carrying NeuroD1. With more ND1 accumulated inside the cells later, see FIG. 3F—Day 7, i.e. 7 dpi, more and more GFP+ cells lose the astrocytic marker (GFAP) and gained the neuronal marker (NeuN), see FIG. 3F and FIG. 3H. Brain sections from animals in the ND1 group were co-immunostained for the immature neuron marker, Tuj1, and the mature neuron marker, MAP2, see FIG. 4B. Most converted neurons show higher Tuj1 and lower MAP2 at early time points, but lower Tuj1 and higher MAP2 at later time points. This could reflect the converted neurons would undergo a maturation process, which may be similar to the developmental stage of the neural stem cells.

Further, astrocyte to neuron conversion was confirmed using a retrovirus vector, which would specifically infect dividing cells and could exclude the leakage issue. The plasmids were constructed to express ND1 under control of a CAG promotor as previously described in Guo et al., Cell Stem Cell, 14:188-202 (2014). Seven days after retrovirus injection, many NeuN+ and GFP+ cells with neuronal morphology were found in brains of mice to which the ND1- and GFP-expressing retrovirus was administered “ND1 retrovirus” group compared to a control expressing GFP only, see FIG. 4C and FIG. 4D.

The Converted Neurons can Develop into Different Subtypes with Cortical Characteristics.

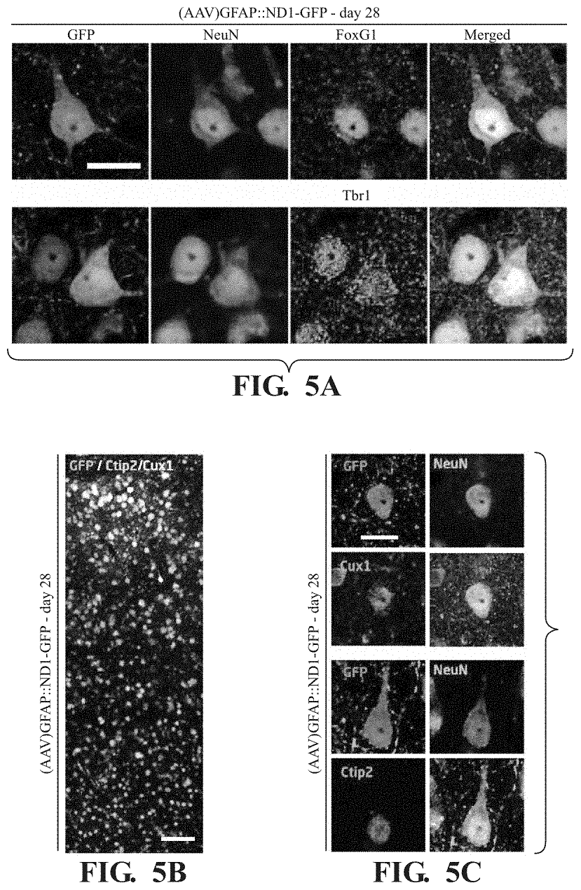

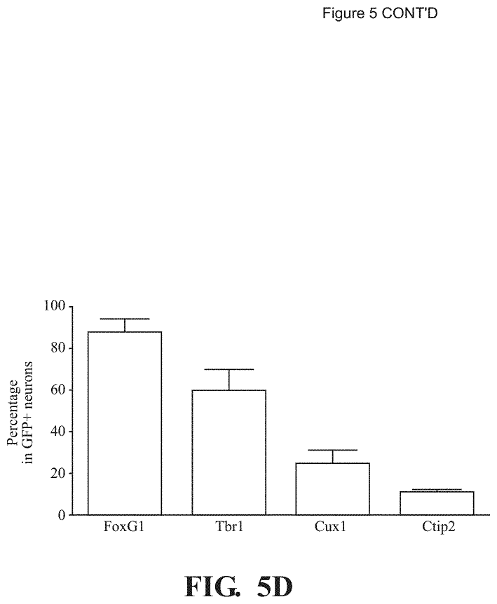

Having shown that glial cells are converted to neurons, it was investigated whether the converted neurons acquire the same molecular profiles as the endogenous neurons. For this study, the cells were immunostained to detect the forebrain marker, Forkhead-box-G1 (FoxG1), and forebrain neuronal marker, T-brain-1 (Tbr1). In mouse brain, FoxG1 is a transcription factor widely spread in all the regions originated from the telencephalon. Tbr1 is involved in neuronal differentiation and migration in mice, especially in glutamatergic neurons. FIGS. 5A, 5B, 5C, and 5D show that the converted neurons could acquire cortical characteristics consistent with local microenvironment.

The results showed that almost all the converted neurons were FoxG1+(88.0%±6.0%, N=3 mice), and the majority of the converted neurons were Tbr1+(59.9%±10.0%, N=3 mice), see FIG. 5A, and FIG. 5D. Further, the cells were immunostained to detect the cortical superficial layer marker Cux1 and deep layer marker Ctip2, see FIG. 5B, and FIG. 5C. After ND1 treatment, these two markers still had the same distribution as in uninjured cortex, see FIG. 5B, and FIG. 5C. In total, 24.6%±6.8% of the GFP+ and NeuN+ cells showed colocalization of Cux1 signal. In the case of Ctip2, the colocalization percentage was 11.0%±1.5%, see FIG. 5D.

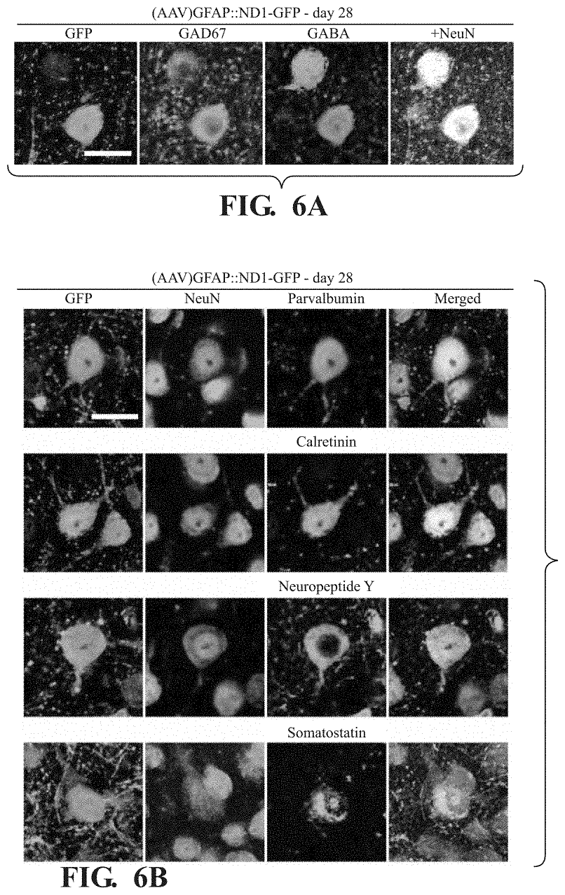

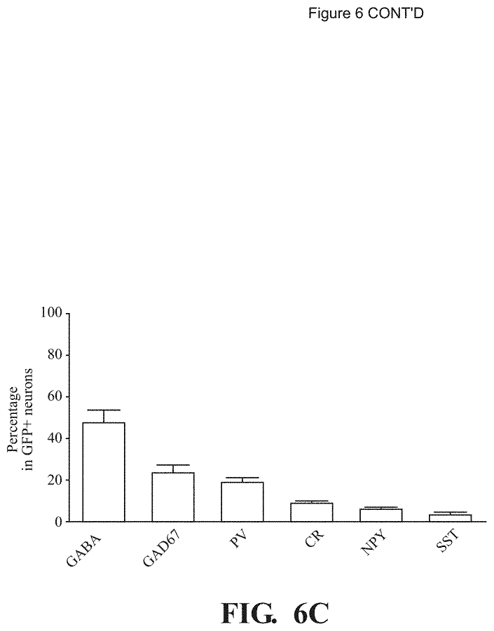

The converted neurons were assayed to determine if they were excitatory or inhibitory neurons. FIGS. 6A, 6B, and 6C show that the converted neurons could differentiate into different subtypes.

By co-immunostaining to detect GABA and GAD67, it was determined that 23.6%±3.5% of GFP+ and NeuN+ cells were immunopositive for both GABA and GAD67, see FIG. 6A and FIG. 6C. Additional studies were performed to determine the subtypes of converted GABAergic neurons by immunostaining to detect the main GABAergic neuron markers, like parvalbumin (PV), calretinin (CR), neuropeptide Y (NPY), somatostatin (SST), choline acetyltransferase (ChAT), and tyrosine hydroxylase (TH), see FIG. 6B. Previous studies showed normal mice cortex had mainly PV, CR, NPY, and SST, no ChAT or TH. In this example, quantification showed that the percentage of PV+ converted neurons reached 19.2%±2.3%, CR+9.1%±0.7%, NPY+7.8%±1.1%, SST+5.3%±3.3%, see FIG. 6C. No converted neurons were found in cortex that colocalized with ChAT or TH. In summary, these results indicated converted neurons differentiated into different subtypes that were consistent with the local microenvironment.

The Converted Neurons are Functionally Mature.

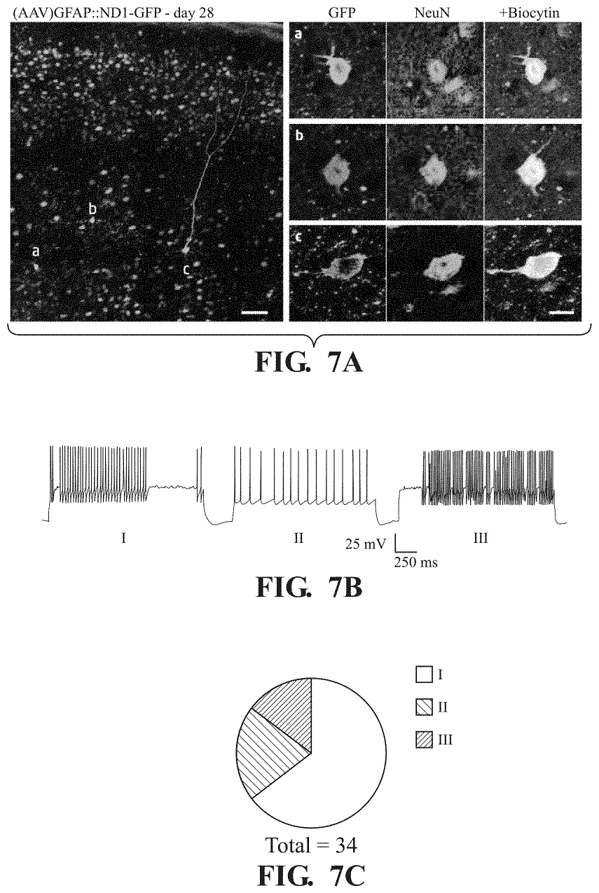

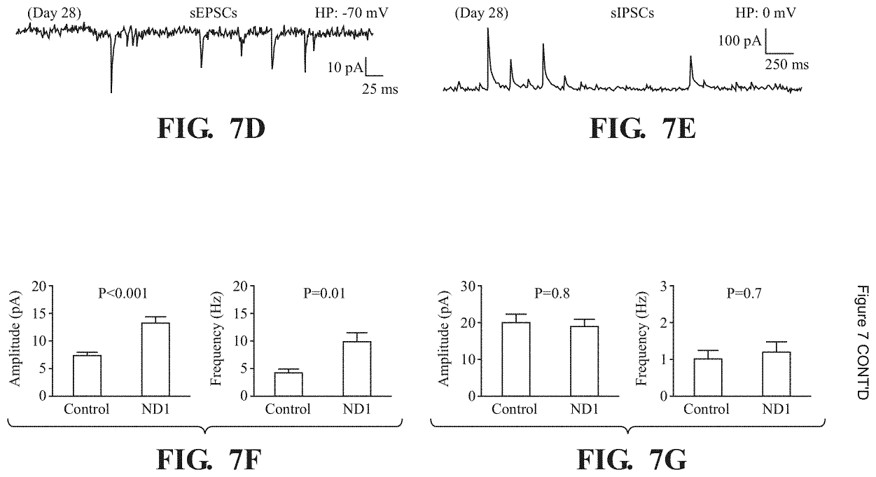

As a fundamental function unit in a brain neural network, each single neuron plays its role by receiving, integrating and transmitting electrical signals. Therefore, the electrophysiological properties of converted neurons were investigated at 4 weeks after virus injection. FIGS. 7A, 7B, 7C, 7D, 7E, 7F, and 7G show that the ND1 converted neurons are functionally mature.

The ability of converted neurons to fire action potentials (APs) was assessed by whole cell recording, and the morphology of the converted neurons was assessed by immunostaining of biotin injected. Three main patterns of firing APs were found, along with different morphologies, see FIG. 7A and FIG. 7B. The first pattern represented about 60 percent of GFP+ neurons recorded, see FIG. 7C. Combined with the representative morphology of neurons with the first firing pattern, see FIG. 7A, it appeared that these might be PV+ interneurons in cortex. Pattern 2 represented about 20 percent of the converted neurons, which might be other interneurons. Converted neurons with pattern 3 were obviously pyramidal neurons. They had long apical dendrites reaching out to superficial layers, and regular firing pattern of Aps, see FIG. 7A and FIG. 7B.

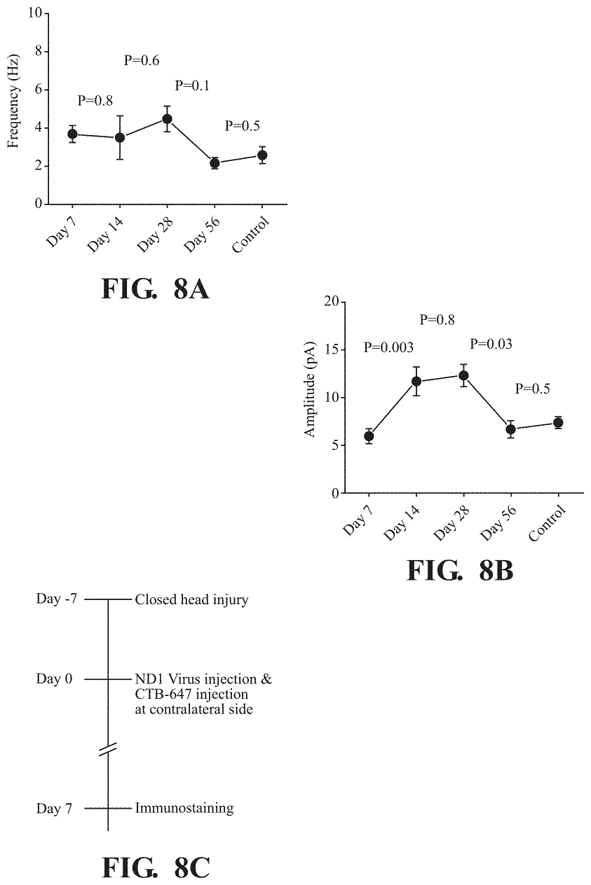

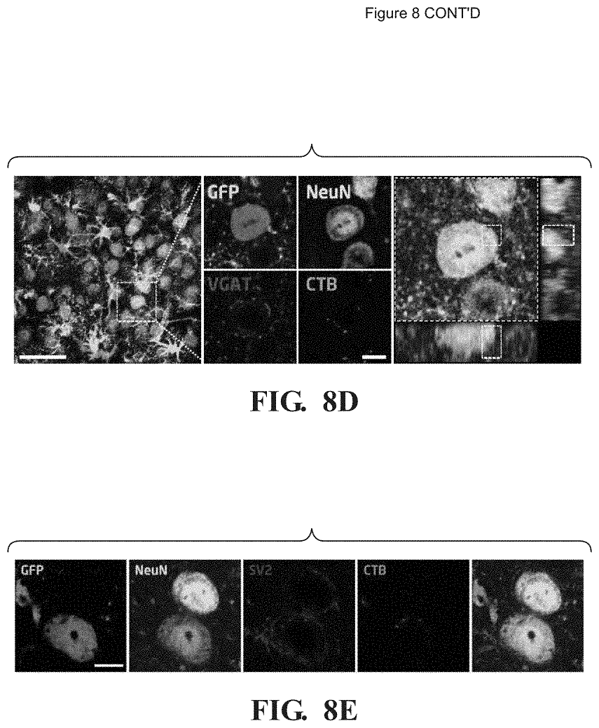

Additional electrophysiological characteristics of converted neurons were then investigated, including spontaneous excitatory postsynaptic currents (sEPSC) and spontaneous inhibitory postsynaptic currents (sIPSC). sEPSC/sIPSC in converted neurons are believed to reflect that the cells are capable of receiving excitatory/inhibitory stimulus from local neural network and giving feedback. At 4 weeks after ND1 treatment, sEPSC and sIPSC were recorded in most GFP+ neurons patched, see FIG. 7D and FIG. 7E, respectively. The amplitude and frequency of sEPSC/sIPSC were compared between the ND1 group and control GFP virus group. There was a significant difference of sEPSC amplitudes between the ND1 group and control GFP virus group (p<0.001), which was respectively 13.3±1.1 pA and 7.4±0.6 pA, see FIG. 7F. The frequency of sEPSC was 6.0±1.0 Hz in ND1 group and 2.6±0.4 Hz in control group with significant difference (p=0.01) between the ND1 group and the control group, see FIG. 7F. In terms of sIPSC, neither the amplitude (ND1: 19.4±1.8 pA, control: 20.4±2.4 pA) nor the frequency (ND1: 1.2±0.3 Hz, control: 1.0±0.2 Hz) had significant difference between the two groups, see FIG. 7G. As the sEPSC showed an evident difference between converted neurons and control, the electrophysiological properties of converted neurons at more time points, including at 1 week, 2 weeks, 4 weeks, and 8 weeks after virus injection (weeks post-injection, wpi), were assessed and compared with control. The frequency of sEPSC in converted neurons was higher at early time points (1, 2, and 4 wpi), then went down at 8 wpi, see FIG. 8A. The amplitude of sEPSC was low at 1 wpi, then went up high at 2 wpi and 4 wpi, and then went down at 8 wpi, see FIG. 8B. Both the frequency and amplitude of sEPSC at the later time point, 8 wpi, were comparable to control, see FIG. 8A and FIG. 8B. The frequency of sEPSC reflected the intensity of excitatory innervations which other neurons put on the converted one. The amplitude of sEPSC could be determined by the density of glutamate receptors on the postsynaptic membrane of converted neurons. These results indicate that converted neurons undergo a structural and functional maturation process similar to that which occurs in neural stem cells.

The Converted Neurons can Integrate into Local Neural Network

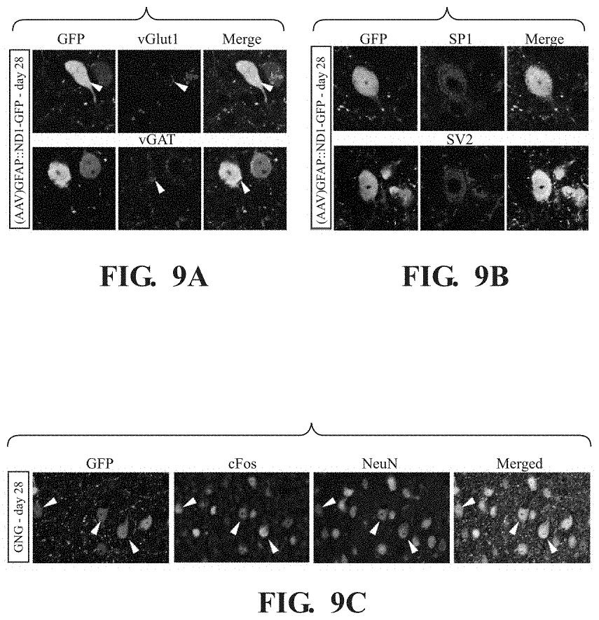

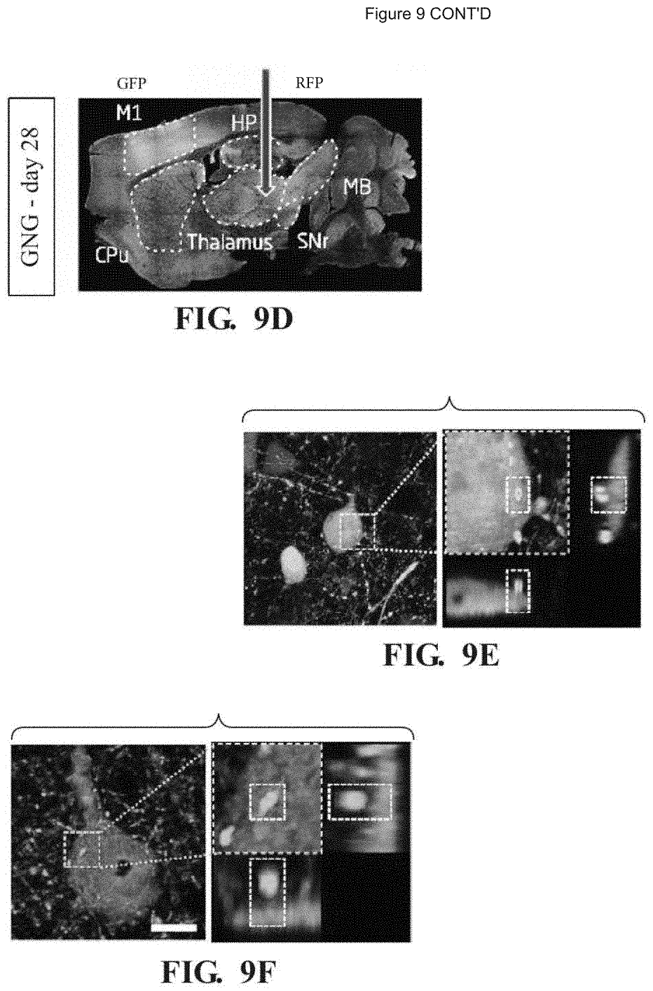

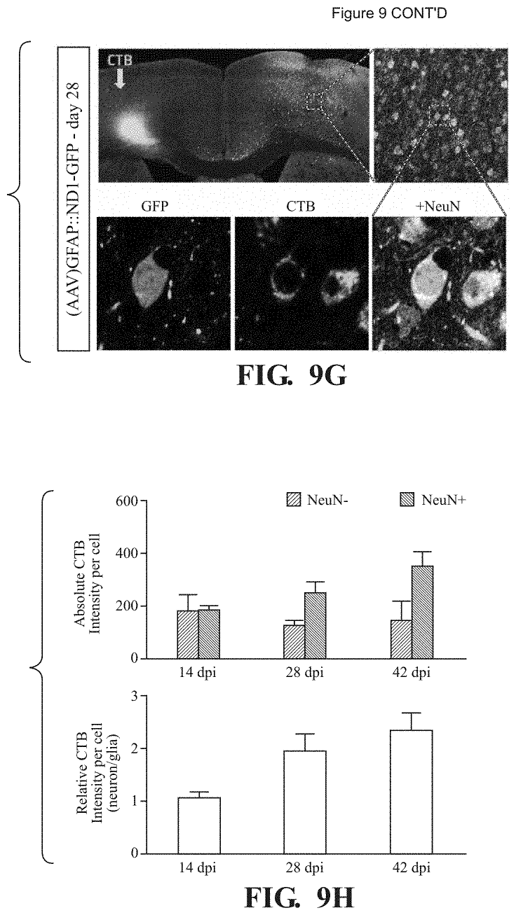

Based on the functional analysis of converted neurons, they were capable of communicating with other neurons in the local neural network, including endogenous neurons and other converted neurons. Therefore, cells were immunostained to detect the synaptic markers, vGlut1 as indicative of excitatory synapses, vGAT as indicative of inhibitory synapses, and synaptophysin (SP1) and synaptic vesicle protein (SV2) as indicative of synaptic transmission. FIGS. 9A, 9B, 9C, 9D, 9E, 9F, and 9H show that ND1-converted neurons integrate into local and remote neural networks.

At 4 wpi, quite a lot converted neurons were found to have vGlut1 or vGAT puncta on the cell soma, which suggested they could receive excitatory or inhibitory inputs from other neurons, see FIG. 9A. Further, converted neurons had expression of SP1 and SV2 inside the soma, especially localized alongside the membrane, indicating ability for synaptic transmission with other neurons, see FIG. 9B.

Next, immunostaining for cFos was performed in mice motor cortex 1 hour after the animals were running on a wheel to check if converted neurons were involved in motor functioning. The imaging data showed that some of the converted neurons had high cFos expression, which was comparable to the endogenous neurons around, see FIG. 9C.

It is well known that neurons receive neural projections not only from other surrounding neurons but also from neurons in remote upstream brain regions. To further investigate whether converted neurons had these remote neural connections built up, anterograde and retrograde tracing were performed.

For the anterograde tracing, viruses, AAV-synapsin::Cre+AAV-CAG::FlexmCherry, labeling neurons were injected in mice thalamus to visualize their axon projections, see FIG. 9D. One week later, the neural projections and synaptic boutons around converted neurons were visualized. It was found that many converted neurons got enlarged synaptic boutons on their soma which showed a signal not only for the anterograde tracing marker but also in for GFP, see FIG. 9E and FIG. 9F, demonstrating that these neurons could receive neural innervations from both remote thalamus neurons and local innervation from other converted neurons.

For the retrograde tracing, Cholera toxin B subunit with fluorophore conjugated (CTB-647) was injected in the contralateral side of the injured motor cortex, see FIG. 8C. Seven days later, the brains of the mice were collected and examined, revealing that some converted neurons had CTB-647 inside the soma in the injury side, see FIG. 9G. This indicated that converted neurons sent distant neural projections to the downstream brain regions as do endogenous neurons. These experiments were repeated at different time points, including 14 days, 28 days, and 42 days following ND1 virus injection. By analyzing the imaging data, the average signal intensity of CTB-647 inside converted neurons was calculated, see FIG. 9H. For absolute CTB-647 signal intensity, there was a significant difference in GFP+ neurons (Day 14: 185±17, Day 28: 249±45, Day 42: 353±50, p=0.01), but not in GFP+ glial cells among different time points (Day 14: 180±61, Day 28: 131±15, Day 42: 150±71, p=0.81). Taking the CTB-647 signal in GFP+ glial cells as background, the relative CTB-647 signal intensity was calculated in GFP+ neurons, which were 1.0±0.1 on Day 14, 1.9±0.3 on Day 28, and 2.3±0.3 on Day 42, with significant difference of p=0.004. The results reflected that the intensity of neural innervations from converted neurons to their downstream brain regions was increasing with time.

FIG. 8D is a set of images illustrating colocalization of a synaptic marker (VGAT) with GFP and NeuN in the cell soma of converted neurons at 7 days after NeuroD1 virus injection and CTB-647 injection on the contralateral side; CTB signal from contralateral side was also observed on the cell soma.

FIG. 8E is a set of images illustrating colocalization of a synaptic vesicle marker (SV2) with GFP and NeuN in the cell soma of converted neurons at 7 days after NeuroD1 virus injection and CTB-647 injection on the contralateral side; CTB signal from contralateral side was also observed on the cell soma.

### Embodiments

Embodiment 1. A method of treating traumatic brain injury (TBI) comprising converting reactive astrocytes to functional neurons by providing exogenous neurogenic differentiation 1 (NeuroD1) to at least one reactive astrocyte in a damaged region of a subject's brain.

Embodiment 2. The method of embodiment 1, wherein the TBI is a closed head injury.

Embodiment 3. The method of embodiments 1 or 2, wherein the damage region of the brain comprises non-functional neurons and reactive astrocytes due to the TBI.

Embodiment 4. The method of embodiment 3, wherein the non-functional neurons are selected from the group consisting of dead and dying neurons.

Embodiment 5. The method of embodiments 3 or 4, wherein the non-functional neurons are detected by a functional MRI (fMRI).

Embodiment 6. The method of any of embodiments 3 to 5, wherein the presence of non-functional neurons and reactive astrocytes in the damaged region are not primarily due to bleeding in the damaged region.

Embodiment 7. The method of any of embodiments 3 to 6, wherein the presence of non-functional neurons and reactive astrocytes in the damaged region are not primarily due to ischemia in the damaged region.

Embodiment 8. The method of any of embodiments 1 to 7, wherein providing the exogenous NeuroD1 comprises administering a recombinant expression vector to the subject, wherein the recombinant expression vector comprises a nucleic acid sequence encoding NeuroD1.

Embodiment 9. The method of any of embodiments 1 to 7, wherein providing the exogenous NeuroD1 comprises administering a recombinant expression vector to the subject, wherein the recombinant expression vector is a viral expression vector comprising a nucleic acid sequence encoding NeuroD1.

Embodiment 10. The method of any of embodiments 1 to 8, wherein providing the exogenous NeuroD1 comprises administering a recombinant expression vector to the subject, wherein the recombinant expression vector is a recombinant adeno-associated virus expression vector, and wherein the recombinant adeno-associated virus vector comprises a nucleic acid sequence encoding NeuroD1.

Embodiment 11. The method of any of embodiments 8 to 10, wherein the nucleic acid sequence encoding NeuroD1 is operably linked to a promoter.

Embodiment 12. The method of embodiment 11, wherein the promoter is a glial-cell specific promoter.

Embodiment 13. The method of embodiment 12, wherein the glial-cell specific promoter is a glial fibrillary acidic protein (GFAP) promoter.

Embodiment 14. The method of embodiment 13, wherein the GFAP promoter is a human GFAP (hGFP) promoter.

Embodiment 15. The method of any of embodiments 1 to 14, wherein no exogenous transcription factor other than NeuroD1 is provided to the at least one reactive astrocyte.

Embodiment 16. The method of any of embodiments 1 to 15, wherein the subject is human.

Embodiment 17. The method of any of embodiments 1 to 16, wherein providing the exogenous NeuroD1 comprises providing exogenous NeuroD1 to the at least one reactive astrocyte at a first treatment time in the range of about two days to about ten days after the traumatic brain injury.

Embodiment 18. The method of any of embodiments 1 to 17, wherein the traumatic brain injury causes a period of astrogliosis in the damaged region, and wherein providing the exogenous NeuroD1 comprises providing exogenous NeuroD1 to the at least one reactive astrocyte at a first treatment time during the period of astrogliosis or within 4 weeks after the period of astrogliosis.

Embodiment 19. The method of embodiment 18, wherein providing the exogenous NeuroD1 comprises providing exogenous NeuroD1 to the at least one reactive astrocyte at a second treatment time after the first treatment time and during the period of astrogliosis or within 4 weeks after the period of astrogliosis.

Embodiment 20. The method of embodiment 19, wherein providing the exogenous NeuroD1 comprises providing exogenous NeuroD1 to the at least one reactive astrocyte at a third treatment time after the second treatment time and during the period of astrogliosis or within 4 weeks after the period of astrogliosis.

Embodiment 21. The method of any of embodiments 1 to 20, wherein the NeuroD1 comprises an amino acid sequence selected from the group consisting of: SEQ ID NO: 2, SEQ ID NO: 4, a functional fragment of SEQ ID NO: 2, a functional fragment of SEQ ID NO: 4, an amino acid sequence having at least 85% identity to SEQ ID NO: 2, and an amino acid sequence having at least 85% identity to SEQ ID NO: 4.

Embodiment 22. The method of embodiment 21, wherein the NeuroD1 is encoded by a nucleic acid sequence comprising SEQ ID NO: 1, a nucleic acid sequence having at least 85% identity to SEQ ID NO: 1, a nucleic acid sequence comprising SEQ ID NO: 3, or a nucleic acid sequence having at least 85% identity to SEQ ID NO: 3.

Embodiment 23. The method of any of embodiments 1 to 22, wherein providing the exogenous NeuroD1 comprises injection into the damaged region of the brain.

Embodiment 24. The method of any of embodiments 8 to 23, wherein the nucleic acid sequence encoding NeuroD1 is present in a virus particle.

Embodiment 25. The method of embodiment 24, wherein providing the exogenous NeuroD1 comprises administering about 10⁷ to about 10¹⁴ virus particles to the damaged brain region of the subject.

Embodiment 26. Use of a composition comprising neurogenic differentiation 1 (NeuroD1) in the manufacture of a medicament for converting reactive astrocytes to functional neurons in a damaged region of a subject's brain, wherein the damaged region of the brain comprises non-functional neurons and reactive astrocytes, due to a traumatic brain injury (TBI).

Embodiment 27. The use of embodiment 26, wherein the non-functional neurons are selected from the group consisting of dead and dying neurons.

Embodiment 28. The use of embodiments 26 or 27, wherein the traumatic brain injury is a closed head injury.

Embodiment 29. The use of any of embodiments 26 to 28, wherein the NeuroD1 is encoded by a nucleic acid sequence comprises a nucleic acid sequence having at least 85% identity to SEQ ID NO: 1.

Embodiment 30. The use of any of embodiments 26 to 29, wherein the nucleic acid encoding NeuroD1 comprises a nucleic acid sequence having at least 85% identity to SEQ ID NO: 3.

Embodiment 31. The use of any of embodiments 26 to 30, wherein the NeuroD1 comprises an amino acid sequence selected from the group consisting of: SEQ ID NO: 2, SEQ ID NO: 4, a functional fragment of SEQ ID NO: 2, a functional fragment of SEQ ID NO: 4, an amino acid sequence having at least 85% identity to SEQ ID NO: 2, and an amino acid sequence having at least 85% identity to SEQ ID NO: 4.

### SEQUENCES

Human NeuroD1 nucleic acid sequence encoding human
NeuroD1 protein-1071 nucleotides, including stop codon
SEQ ID NO: 1
ATGACCAAATCGTACAGCGAGAGTGGGCTGATGGGCGAGCCTCAGCCCCAAGGTCC
TCCAAGCTGGACAGACGAGTGTCTCAGTTCTCAGGACGAGGAGCACGAGGCAGAC
AAGAAGGAGGACGACCTCGAAGCCATGAACGCAGAGGAGGACTCACTGAGGAACG
GGGGAGAGGAGGAGGACGAAGATGAGGACCTGGAAGAGGAGGAAGAAGAGGAAG
AGGAGGATGACGATCAAAAGCCCAAGAGACGCGGCCCCAAAAAGAAGAAGATGAC
TAAGCCTCCCCTGGAGCGTTTTAAATTGAGACGCATGAAGGCTAACGCCCGGGAGC
GGAACCGCATGCACGGACTGAACGCGGCGCTAGACAACCTGCGCAAGGTGGTGCCT
TGCTATTCTAAGACGCAGAAGCTGTCCAAAATCGAGACTCTGCGCTTGGCCAAGAAC
TACATCTGGGCTCTGTCGGAGATCCTGCGCTCAGGCAAAAGCCCAGACCTGGTCTC
CTTCGTTCAGACGCTTTGCAAGGGCTTATCCCAACCCACCACCAACCTGGTTGCGGG
CTGCCTGCAACTCAATCCTCGGACTTTTCTGCCTGAGCAGAACCAGGACATGCCCCC
CCACCTGCCGACGGCCAGCGCTTCCTTCCCTGTACACCCCTACTCCTACCAGTCGCC
TGGGCTGCCCAGTCCGCCTTACGGTACCATGGACAGCTCCCATGTCTTCCACGTTAA
GCCTCCGCCGCACGCCTACAGCGCAGCGCTGGAGCCCTTCTTTGAAAGCCCTCTGAC
TGATTGCACCAGCCCTTCCTTTGATGGACCCCTCAGCCCGCCGCTCAGCATCAATGG
CAACTTCTCTTTCAAACACGAACCGTCCGCCGAGTTTGAGAAAAATTATGCCTTTAC
CATGCACTATCCTGCAGCGACACTGGCAGGGGCCCAAAGCCACGGATCAATCTTCTC
AGGCACCGCTGCCCCTCGCTGCGAGATCCCCATAGACAATATTATGTCCTTCGATAG
CCATTCACATCATGAGCGAGTCATGAGTGCCCAGCTCAATGCCATATTTCATGATTA
G
Human NeuroD1 amino acid sequence-356 amino acids-
encoded by SEQ ID NO: 1
SEQ ID NO: 2
MTKSYSESGLMGEPQPQGPPSWTDECLSSQDEEHEADKKEDDLEAMNAEEDSLRNGGE
EEDEDEDLEEEEEEEEEDDDQKPKRRGPKKKKMTKARLERFKLRRMKANARERNRMH
GLNAALDNLRKVVPCYSKTQKLSKIETLRLAKNYIWALSEILRSGKSPDLVSFVQTLCK
GLSQPTTNLVAGCLQLNPRTFLPEQNQDMPPHLPTASASFPVHPYSYQSPGLPSPPYGT
MDSSHVFHVKPPPHAYSAALEPFFESPLTDCTSPSFDGPLSPPLSINGNFSFKJEPSAEFEK
NTAFTMHYPAATLAGAQSHGSIFSGTAAPRCEIPIDNIMSFDSHSHHERVMSAQLNAIFH
D
Mouse NeuroD1 nucleic acid sequence encoding mouse NeuroD1
protein-1074 nucleotides, including stop codon
SEQ ID NO: 3
ATGACCAAATCATACAGCGAGAGCGGGCTGATGGGCGAGCCTCAGCCCCAAGGTCC
CCCAAGCTGGACAGATGAGTGTCTCAGTTCTCAGGACGAGGAACACGAGGCAGAC
AAGAAAGAGGACGAGCTTGAAGCCATGAATGCAGAGGAGGACTCTCTGAGAAACG
GGGGAGAGGAGGAGGAGGAAGATGAGGATCTAGAGGAAGAGGAGGAAGAAGAAG
AGGAGGAGGAGGATCAAAAGCCCAAGAGACGGGGTCCCAAAAAGAAAAAGATGA
CCAAGGCGCGCCTAGAACGTTTTAAATTAAGGCGCATGAAGGCCAACGCCCGCGAG
CGGAACCGCATGCACGGGCTGAACGCGGCGCTGGACAACCTGCGCAAGGTGGTAC
CTTGCTACTCCAAGACCCAGAAACTGTCTAAAATAGAGACACTGCGCTTGGCCAAG
AACTACATCTGGGCTCTGTCAGAGATCCTGCGCTCAGGCAAAAGCCCTGATCTGGT
CTCCTTCGTACAGACGCTCTGCAAAGGTTTGTCCCAGCCCACTACCAATTTGGTCGC
CGGCTGCCTGCAGCTCAACCCTCGGACTTTCTTGCCTGAGCAGAACCCGGACATGCC
CCCGCATCTGCCAACCGCCAGCGCTTCCTTCCCGGTGCATCCCTACTCCTACCAGTC
CCCTGGACTGCCCAGCCCGCCCTACGGCACCATGGACAGCTCCCACGTCTTCCACGT
CAAGCCGCCGCCACACGCCTACAGCGCAGCTCTGGAGCCCTTCTTTGAAAGCCCCC
TAACTGACTGCACCAGCCCTTCCTTTGACGGACCCCTCAGCCCGCCGCTCAGCATCA
ATGGCAACTTCTCTTTCAAACACGAACCATCCGCCGAGTTTGAAAAAAATTATGCCT
TTACCATGCACTACCCTGCAGCGACGCTGGCAGGGCCCCAAAGCCACGGATCAATC
TTCTCTTCCGGTGCCGCTGCCCCTCGCTGCGAGATCCCCATAGACAACATTATGTCT
TTCGATAGCCATTCGCATCATGAGCGAGTCATGAGTGCCCAGCTTAATGCCATCTTT
CACGATTAG
Mouse NeuroD1 amino acid sequence-357 amino acids-
encoded by SEQ ID NO: 3
SEQ ID NO: 4
MTKSYSESGLMGEPQPQGPPSWTDECLSSQDEEHEADKKEDELEAMNAEEDSLRNGGE
EEEEDEDLEEEEEEEEEEEDQKPKRRGPKKKKMTKARLERFKLRRMKANARERNRMH
GLNAALDNLRKVVPCYSKTQKLSKIETLRLAKNYIWALSEILRSGKSPDLVSFVQTLCK
GLSQPTTNLVAGCLQLNPRTFLPEQNPDMPPHLPTASASFPVHPYSYQSPGLPSPPYGTM
DSSHVFHVKPPPHAYSAALEPFFESPLTDCTSPSFDGPLSPPLSINGNFSFKHEPSAEFEKN
YAFTMHYPAATLAGPQSHGSIFSSGAAAPRCEIPIDNIMSFDSHSHHERVMSAQLNAIFH
D
LCN2 Promoter
SEQ ID NO: 5
GCAGTGTGGAGACACACCCACTTTCCCCAAGGGCTCCTGCTCCCCCAAGTGATCCCC
TTATCCTCCGTGCTAAGATGACACCGAGGTTGCAGTCCTTACCTTTGAAAGCAGCCA
CAAGGGCGTGGGGGTGCACACCTTTAATCCCAGCACTCGGGAGGCAGAGGCAGGC
AGATTTCTGAGTTCGAGACCAGCCTGGTCTACAAAGTGAATTCCAGGACAGCCAGG
GCTATACAGAGAAACCCTGTCTTGAAAAAAAAAGAGAAAGAAAAAAGAAAAAAAA
AAATGAAAGCAGCCACATCTAAGGACTACGTGGCACAGGAGAGGGTGAGTCCCTGA
GAGTTCAGCTGCTGCCCTGTCTGTTCCTGTAAATGGCAGTGGGGTCATGGGAAAGTG
AAGGGGCTCAAGGTATTGGACACTTCCAGGATAATCTTTTGGACGCCTCACCCTGTG
CCAGGACCAAGGCTGAGCTTGGCAGGCTCAGAACAGGGTGTCCTGTTCTTCCCTGTC
TAAAACATTCACTCTCAGCTTGCTCACCCTTCCCCAGACAAGGAAGCTGCACAGGG
TCTGGTGTTCAGATGGCTTTGGCTTACAGCAGGTGTGGGTGTGGGGTAGGAGGCAGG
GGGTAGGGGTGGGGGAAGCCTGTACTATACTCACTATCCTGTTTCTGACCCTCTAGG
ACTCCTACAGGGTTATGGGAGTGGACAGGCAGTCCAGATCTGAGCTGCTGACCCAC
AAGCAGTGCCCTGTGCCTGCCAGAATCCAAAGCCCTGGGAATGTCCCTCTGGTCCCC
CTCTGTCCCCTGCAGCCCTTCCTGTTGCTCAACCTTGCACAGTTCCGACCTGGGGGA
GAGAGGGACAGAAATCTTGCCAAGTATTTCAACAGAATGTACTGGCAATTACTTCAT
GGCTTCCTGGACTTGGTAAAGGATGGACTACCCCGCCCAACAGGGGGGCTGGCAGC
CAGGTAGGCCCATAAAAAGCCCGCTGGGGAGTCCTCCTCACTCTCTGCTCTTCCTCC
TCCAGCACACATCAGACCTAGTAGCTGTGGAAACCA
Human GFAP Promoter
SEQ ID NO: 6
GTCTGCAAGCAGACCTGGCAGCATTGGGCTGGCCGCCCCCCAGGGCCTCCTCTTCAT
GCCCAGTGAATGACTCACCTTGGCACAGACACAATGTTCGGGGTGGGCACAGTGCC
TGCTTCCCGCCGCACCCCAGCCCCCCTCAAATGCCTTCCGAGAAGCCCATTGAGTAG
GGGGCTTGCATTGCACCCCAGCCTGACAGCCTGGCATCTTGGGATAAAAGCAGCAC
AGCCCCCTAGGGGCTGCCCTTGCTGTGTGGCGCCACCGGCGGTGGAGAACAAGGCT
CTATTCAGCCTGTGCCCAGGAAAGGGGATCAGGGGATGCCCAGGCATGGACAGTGG
GTGGCAGGGGGGGAGAGGAGGGCTGTCTGCTTCCCAGAAGTCCAAGGACACAAATG
GGTGAGGGGACTGGGCAGGGTTCTGACCCTGTGGGACCAGAGTGGAGGGCGTAGAT
GGACCTGAAGTCTCCAGGGACAACAGGGCCCAGGTCTCAGGCTCCTAGTTGGGCCC
AGTGGCTCCAGCGTTTCCAAACCCATCCATCCCCAGAGGTTCTTCCCATCTCTCCAG
GCTGATGTGTGGGAACTCGAGGAAATAAATCTCCAGTGGGAGACGGAGGGGTGGCC
AGGGAAACGGGGCGCTGCAGGAATAAAGACGAGCCAGCACAGCCAGCTCATGCGT
AACGGCTTTGTGGAGCTGTCAAGGCCTGGTCTCTGGGAGAGAGGCACAGGGAGGCC
AGACAAGGAAGGGGTGACCTGGAGGGACAGATCCAGGGGCTAAAGTCCTGATAAG
GCAAGAGAGTGCCGGCCCCCTCTTGCCCTATCAGGACCTCCACTGCCACATAGAGGC
CATGATTGACCCTTAGACAAAGGGCTGGTGTCCAATCCCAGCCCCCAGCCCCAGAA
CTCCAGGGAATGAATGGGCAGAGAGCAGGAATGTGGGACATCTGTGTTCAAGGGAA
GGACTCCAGGAGTCTGCTGGGAATGAGGCCTAGTAGGAAATGAGGTGGCCCTTGAG
GGTACAGAACAGGTTCATTCTTCGCCAAATTCCCAGCACCTTGCAGGCACTTACAGC
TGAGTGAGATAATGCCTGGGTTATGAAATCAAAAAGTTGGAAAGCAGGTCAGAGGT
CATCTGGTACAGCCCTTCCTTCCCTTTTTTTTTTTTTTTTTTTGTGAGACAAGGTCTCT
CTCTGTTGCCCAGGCTGGAGTGGCGCAAACACAGCTCACTGCAGCCTCAACCTACTG
GGCTCAAGCAATCCTCCAGCCTCAGCCTCCCAAAGTGCTGGGATTACAAGCATGAG
CCACCCCACTCAGCCCTTTCCTTCCTTTTTAATTGATGCATAATAATTGTAAGTATTC
ATCATGGTCCAACCAACCCTTTCTTGACCCACCTTCCTAGAGAGAGGGTCCTCTTGA
TTCAGCGGTCAGGGCCCCAGACCCATGGTCTGGCTCCAGGTACCACCTGCCTCATGC
AGGAGTTGGCGTGCCCAGGAAGCTCTGCCTCTGGGCACAGTGACCTCAGTGGGGTG
AGGGGAGCTCTCCCCATAGCTGGGCTGCGGCCCAACCCCACCCCCTCAGGCTATGCC
AGGGGGTGTTGCCAGGGGCACCCGGGCATCGCCAGTCTAGCCCACTCCTTCATAAA
GCCCTCGCATCCCAGGAGCGAGCAGAGCCAGAGCAT
Mouse Aldh1L1 promoter
SEQ ID NO: 7
AACTGAGAGTGGAGGGGCACAGAAGAGCCCAAGAGGCTCCTTAGGTTGTGTGGAGG
GTACAATATGTTTGGGCTGAGCAACCCAGAGCCAGACTTTGTCTGGCTGGTAAGAGA
CAGAGGTGCCTGCTATCACAATCCAAGGGTCTGCTTGAGGCAGAGCCAGTGCAAAG
GATGTGGTTAGAGCCAGCCTGGTGTACTGAAGAGGGGCGAAGAGCTTGAGTAAGG
AGTCTCAGCGGTGGTTTGAGAGGCAGGGTGGTTAATGGAGTAGCTGCAGGGGAGAA
TCCTTGGGAGGGAGCCTGCAGGACAGAGCTTTGGTCAGGAAGTGATGGGCATGTCA
CTGGACCCTGTATTGTCTCTGACTTTTCTCAAGTAGGACAATGACTCTGCCCAGGGA
GGGGGTCTGTGACAAGGTGGAAGGGCCAGAGGAGAACTTCTGAGAAGAAAACCAG
AGGCCGTGAAGAGGTGGGAAGGGCATGGGATTCAGAACCTCAGGCCCACCAGGAC
ACAACCCCAGGTCCACAGCAGATGGGTGACCTTGCATGTCTCAGTCACCAGCATTGT
GCTCCTTGCTTATCACGCTTGGGTGAAGGAAATGACCCAAATAGCATAAAGCCTGAA
GGCCGGGACTAGGCCAGCTAGGGCTTGCCCTTCCCTTCCCAGCTGCACTTTCCATAG
GTCCCACCTTCAGCAGATTAGACCCGCCTCCTGCTTCCTGCCTCCTTGCCTCCTCACT
CATGGGTCTATGCCCACCTCCAGTCTCGGGACTGAGGCTCACTGAAGTCCCATCGAG
GTCTGGTCTGGTGAATCAGCGGCTGGCTCTGGGCCCTGGGCGACCAGTTAGGTTCCG
GGCATGCTAGGCAATGAACTCTACCCGGAATTGGGGGTGCGGGGAGGCGGGGAGGT
CTCCAACCCAGCCTTTTGAGGACGTGCCTGTCGCTGCACGGTGCTTTTTATAGACGA
TGGTGGCCCATTTTGCAGAAGGGAAAGCCGGAGCCCTCTGGGGAGCAAGGTCCCCG
CAAATGGACGGATGACCTGAGCTTGGTTCTGCCAGTCCACTTCCCAAATCCCTCACC
CCATTCTAGGGACTAGGGAAAGATCTCCTGATTGGTCATATCTGGGGGCCTGGCCGG
AGGGCCTCCTATGATTGGAGAGATCTAGGCTGGGCGGGCCCTAGAGCCCGCCTCTTC
TCTGCCTGGAGGAGGAGCACTGACCCTAACCCTCTCTGCACAAGACCCGAGCTTGTG
CGCCCTTCTGGGAGCTTGCTGCCCCTGTGCTGACTGCTGACAGCTGACTGACGCTCG
CAGCTAGCAGGTACTTCTGGGTTGCTAGCCCAGAGCCCTGGGCCGGTGACCCTGTTT
TCCCTACTTCCCGTCTTTGACCTTGGGTAAGTTTCTTTTTCTTTTGTTTTTGAGAGAGG
CACCCAGATCCTCTCCACTACAGGCAGCCGCTGAACCTTGGATCCTCAGCTCCTGCC
CTGGGAACTACAGTTCCTGCCCTTTTTTTCCCACCTTGAGGGAGGTTTTCCCTGAGTA
GCTTCGACTATCCTGGAACAAGCTTTGTAGACCAGCCTGGGTCTCCGGAGAGTTGGG
ATTAAAGGCGTGCACCACCACC
Human NG2 promoter
SEQ ID NO: 8
CTCTGGTTTCAAGACCAATACTCATAACCCCCACATGGACCAGGCACCATCACACCT
GAGCACTGCACTTAGGGTCAAAGACCTGGCCCCACATCTCAGCAGCTATGTAGACT
AGCTCCAGTCCCTTAATCTCTCTCAGCCTCAGTTTCTTCATCTGCAAAACAGGTCTCA
GTTTCGTTGCAAAGTATGAAGTGCTGGGCTGTTACTGGTCAAAGGGAAGAGCTGGG
AAGAGGGTGCAAGGTGGGGTTGGGCTGGAGATGGGCTGGAGCAGATAGATGGAGG
GACCTGAATGGAGGAAGTAAACCAAGGCCCGGTAACATTGGGACTGGACAGAGAA
CACGCAGATCCTCTAGGCACCGGAAGCTAAGTAACATTGCCCTTTCTCCTCCTGTTT
GGGACTAGGCTGATGTTGCTGCCTGGAAGGGAGCCAGCAGAAGGGCCCCAGCCTG
AAGCTGTTAGGTAGAAGCCAAATCCAGGGCCAGATTTCCAGGAGGCAGCCTCGGGA
AGTTGAAACACCCGGATTCAGGGGTCAGGAGGCCTGGGCTTCTGGCACCAAACGGC
CAGGGACCTACTTTCCACCTGGAGTCTTGTAAGAGCCACTTTCAGCTTGAGCTGCAC
TTTCGTCCTCCATGAAATGGGGGAGGGGATGCTCCTCACCCACCTTGCAAGGTTATT
TTGAGGCAAATGTCATGGCGGGACTGAGAATTCTTCTGCCCTGCGAGGAAATCCAG
ACATCTCTCCCTTACAGACAGGGAGACTGAGGTGAGGCCCTTCCAGGCAGAGAAGG
TCACTGTTGCAGCCATGGGCAGTGCCCCACAGGACCTCGGGTGGTGCCTCTGGAGTC
TGGAGAAGTTCCTAGGGGACCTCCGAGGCAAAGCAGCCCAAAAGCCGCCTGTGAGG
GTGGCTGGTGTCTGTCCTTCCTCCTAAGGCTGGAGTGTGCCTGTGGAGGGGTCTCCT
GAACTCCCGCAAAGGCAGAAAGGAGGGAAGTAGGGGCTGGGACAGTTCATGCCTCC
TCCCTGAGGGGGTCTCCCGGGCTCGGCTCTTGGGGCCAGAGTTCAGGGTGTCTGGGC
CTCTCTATGACTTTGTTCTAAGTCTTTAGGGTGGGGCTGGGGTCTGGCCCAGCTGCA
AGGGCCCCCTCACCCCTGCCCCAGAGAGGAACAGCCCCGCACGGGCCCTTTAAGAA
GGTTGAGGGTGGGGGCAGGTGGGGGAGTCCAAGCCTGAAACCCGAGCGGGCGCGC
GGGTCTGCGCCTGCCCCGCCCCCGGAGTTAAGTGCGCGGACACCCGGAGCCGGCCC
GCGCCCAGGAGCAGAGCCGCGCTCGCTCCACTCAGCTCCCAGCTCCCAGGACTCCG
CTGGCTCCTCGCAAGTCCTGCCGCCCAGCCCGCCGGG
CAG::NeuroD1-IRES-GFP
SEQ ID NO: 9
GATCCGGCCATTAGCCATATTATTCATTGGTTATATAGCATAAATCAATATTGGCTAT
TGGCCATTGCATACGTTGTATCCATATCATAATATGTACATTTATATTGGCTCATGTC
CAACATTACCGCCATGTTGACATTGATTATTGACTAGTTATTAATAGTAATCAATTAC
GGGGTCATTAGTTCATAGCCCATATATGGAGTTCCGCGTTACATAACTTACGGTAAA
TGGCCCGCCTGGCTGACCGCCCAACGACCCCCGCCCATTGACGTCAATAATGACGTA
TGTTCCCATAGTAACGCCAATAGGGACTTTCCATTGACGTCAATGGGTGGAGTATTT
ACGGTAAACTGCCCACTTGGCAGTACATCAAGTGTATCATATGCCAAGTACGCCCCC
TATTGACGTCAATGACGGTAAATGGCCCGCCTGGCATTATGCCCAGTACATGACCTT
ATGGGACTTTCCTACTTGGCAGTACATCTACGTATTAGTCATCGCTATTACCATGGTG
ATGCGGTTTTGGCAGTACATCAATGGGCGTGGATAGCGGTTTGACTCACGGGGATTT
CCAAGTCTCCACCCCATTGACGTCAATGGGAGTTTGTTTTGGCACCAAAATCAACGG
GACTTTCCAAAATGTCGTAACAACTCCGCCCCATTGACGCAAATGGGCGGTAGGCAT
GTACGGTGGGAGGTCTATATAAGCAGAGCTCAATAAAAGAGCCCACAACCCCTCAC
TCGGGGCGCCAGTCCTCCGATTGACTGAGTCGCCCGGGTACCCGTATTCCCAATAAA
GCCTCTTGCTGTTTGCATCCGAATCGTGGTCTCGCTGTTCCTTGGGAGGGTCTCCTCT
GAGTGATTGACTACCCACGACGGGGGTCTTTCATTTGGGGGCTCGTCCGGGATTTGG
AGACCCCTGCCCAGGGACCACCGACCCACCACCGGGAGGTAAGCTGGCCAGCAACT
TATCTGTGTCTGTCCGATTGTCTAGTGTCTATGTTTGATGTTATGCGCCTGCGTCTGT
ACTAGTTAGCTAACTAGCTCTGTATCTGGCGGACCCGTGGTGGAACTGACGAGTTCT
GAACACCCGGCCGCAACCCTGGGAGACGTCCCAGGGACTTTGGGGGCCGTTTTTGT
GGCCCGACCTGAGGAAGGGAGTCGATGTGGAATCCGACCCCGTCAGGATATGTGGT
TCTGGTAGGAGACGAGAACCTAAAACAGTTCCCGCCTCCGTCTGAATTTTTGCTTTC
GGTTTGGAACCGAAGCCGCGCGTCTTGTCTGCTGCAGCGCTGCAGCATCGTTCTGTG
TTGTCTCTGTCTGACTGTGTTTCTGTATTTGTCTGAAAATTAGGGCCAGACTGTTACC
ACTCCCTTAAGTTTGACCTTAGGTCACTGGAAAGATGTCGAGCGGATCGCTCACAAC
CAGTCGGTAGATGTCAAGAAGAGACGTTGGGTTACCTTCTGCTCTGCAGAATGGCCA
ACCTTTAACGTCGGATGGCCGCGAGACGGCACCTTTAACCGAGACCTCATCACCCAG
GTTAAGATCAAGGTCTTTTCACCTGGCCCGCATGGACACCCAGACCAGGTCCCCTAC
ATCGTGACCTGGGAAGCCTTGGCTTTTGACCCCCCTCCCTGGGTCAAGCCCTTTGTA
CACCCTAAGCCTCCGCCTCCTCTTCCTCCATCCGCCCCGTCTCTCCCCCTTGAACCTC
CTCGTTCGACCCCGCCTCGATCCTCCCTTTATCCAGCCCTCACTCCTTCTCTAGGCGC
CGGAATTCGATGTCGACATTGATTATTGACTAGTTATTAATAGTAATCAATTACGGG
GTCATTAGTTCATAGCCCATATATGGAGTTCCGCGTTACATAACTTACGGTAAATGG
CCCGCCTGGCTGACCGCCCAACGACCCCCGCCCATTGACGTCAATAATGACGTATGT
TCCCATAGTAACGCCAATAGGGACTTTCCATTGACGTCAATGGGTGGACTATTTACG
GTAAACTGCCCACTTGGCAGTACATCAAGTGTATCATATGCCAAGTACGCCCCCTAT
TGACGTCAATGACGGTAAATGGCCCGCCTGGCATTATGCCCAGTACATGACCTTATG
GGACTTTCCTACTTGGCAGTACATCTACGTATTAGTCATCGCTATTACCATGGGTCGA
GGTGAGCCCCACGTTCTGCTTCACTCTCCCCATCTCCCCCCCCTCCCCACCCCCAATT
TTGTATTTATTTATTTTTTAATTATTTTGTGCAGCGATGGGGGCGGGGGGGGGGGGG
GCGCGCGCCAGGCGGGGCGGGGCGGGGCGAGGGGCGGGGCGGGGCGAGGCGGAG
AGGTGCGGCGGCAGCCAATCAGAGCGGCGCGCTCCGAAAGTTTCCTTTTATGGCGA
GGCGGCGGCGGCGGCGGCCCTATAAAAAGCGAAGCGCGCGGCGGGCGGGAGTCGC
TGCGTTGCCTTCGCCCCGTGCCCCGCTCCGCGCCGCCTCGCGCCGCCCGCCCCGGCT
CTGACTGACCGCGTTACTCCCACAGGTGAGCGGGCGGGACGGCCCTTCTCCTCCGGG
CTGTAATTAGCGCTTGGTTTAATGACGGCTCGTTTCTTTTCTGTGGCTGCGTGAAAGC
CTTAAAGGGCTCCGGGAGGGCCCTTTGTGCGGGGGGGAGCGGCTCGGGGGGTGCGT
GCGTGTGTGTGTGCGTGGGGAGCGCCGCGTGCGGCCCGCGCTGCCCGGCGGCTGTG
AGCGCTGCGGGCGCGGCGCGGGGCTTTGTGCGCTCCGCGTGTGCGCGAGGGGAGCG
CGGCCGGGGGCGGTGCCCCGCGGTGCGGGGGGGCTGCGAGGGGAACAAAGGCTGC
GTGCGGGGTGTGTGCGTGGGGGGGTGAGCAGGGGGTGTGGGCGCGGCGGTCGGGCT
GTAACCCCCCCCTGCACCCCCCTCCCCGAGTTGCTGAGCACGGCCCGGCTTCGGGTG
CGGGGCTCCGTGCGGGGCGTGGCGCGGGGCTCGCCGTGCCGGGCGGGGGGTGGCGG
CAGGTGGGGGTGCCGGGCGGGGCGGGGCCGCCTCGGGCCGGGGAGGGCTCGGGGG
AGGGGCGCGGCGGCCCCGGAGCGCCGGCGGCTGTCGAGGCGCGGCGAGCCGCAGC
CATTGCCTTTTATGGTAATCGTGCGAGAGGGCGCAGGGACTTCCTTTGTCCCAAATC
TGGCGGAGCCGAAATCTGGGAGGCGCCGCCGCACCCCCTCTAGCGGGCGCGGGCGA
AGCGGTGCGGCGCCGGCAGGAAGGAAATGGGCGGGGAGGGCCTTCGTGCGTCGCC
GCGCCGCCGTCCCCTTCTCCATCTCCAGCCTCGGGGCTGCCGCAGGGGGACGGCTGC
CTTCGGGGGGGACGGGGCAGGGCGGGGTTCGGCTTCTGGCGTGTGACCGGCGGCTC
TAGAGCCTCTGCTAACCATGTTCATGCCTTCTTCTTTTTCCTACAGCTCCTGGGCAAC
GTGCTGGTTGTTGTGCTGTCTCATCATTTTGGCAAAGAATTCGCTAGCGGATCCGGC
CGCCTCGGCCACCGGTCGCCACCATCGCCACCATGACCAAATCATACAGCGAGAGC
GGGCTGATGGGCGAGCCTCAGCCCCAAGGTCCCCCAAGCTGGACAGATGAGTGTCT
CAGTTCTCAGGACGAGGAACACGAGGCAGACAAGAAAGAGGACGAGCTTGAAGCC
ATGAATGCAGAGGAGGACTCTCTGAGAAACGGGGGAGAGGAGGAGGAGGAAGATG
AGGATCTAGAGGAAGAGGAGGAAGAAGAAGAGGAGGAGGAGGATCAAAAGCCCA
AGAGACGGGGTCCCAAAAAGAAAAAGATGACCAAGGCGCGCCTAGAACGTTTTAA
ATTAAGGCGCATGAAGGCCAACGCCCGCGAGCGGAACCGCATGCACGGGCTGAACG
CGGCGCTGGACAACCTGCGCAAGGTGGTACCTTGCTACTCCAAGACCCAGAAACTG
TCTAAAATAGAGACACTGCGCTTGGCCAAGAACTACATCTGGGCTCTGTCAGAGATC
CTGCGCTCAGGCAAAAGCCCTGATCTGGTCTCCTTCGTACAGACGCTCTGCAAAGGT
TTGTCCCAGCCCACTACCAATTTGGTCGCCGGCTGCCTGCAGCTCAACCCTCGGACT
TTCTTGCCTGAGCAGAACCCGGACATGCCCCCGCATCTGCCAACCGCCAGCGCTTCC
TTCCCGGTGCATCCCTACTCCTACCAGTCCCCTGGACTGCCCAGCCCGCCCTACGGC
ACCATGGACAGCTCCCACGTCTTCCACGTCAAGCCGCCGCCACACGCCTACAGCGCA
GCTCTGGAGCCCTTCTTTGAAAGCCCCCTAACTGACTGCACCAGCCCTTCCTTTGAC
GGACCCCTCAGCCCGCCGCTCAGCATCAATGGCAACTTCTCTTTCAAACACGAACCA
TCCGCCGAGTTTGAAAAAAATTATGCCTTTACCATGCACTACCCTGCAGCGACGCTG
GCAGGGCCCCAAAGCCACGGATCAATCTTCTCTTCCGGTGCCGCTGCCCCTCGCTGC
GAGATCCCCATAGACAACATTATGTCTTTCGATAGCCATTCGCATCATGAGCGAGTC
ATGAGTGCCCAGCTTAATGCCATCTTTCACGATTAGGTTTAAACGCGGCCGCGCCCC
TCTCCCTCCCCCCCCCCTAACGTTACTGGCCGAAGCCGCTTGGAATAAGGCCGGTGT
GCGTTTGTCTATATGTTATTTTCCACCATATTGCCGTCTTTTGGCAATGTGAGGGCCC
GGAAACCTGGCCCTGTCTTCTTGACGAGCATTCCTAGGGGTCTTTCCCCTCTCGCCA
AAGGAATGCAAGGTCTGTTGAATGTCGTGAAGGAAGCAGTTCCTCTGGAAGCTTCTT
GAAGACAAACAACGTCTGTAGCGACCCTTTGCAGGCAGCGGAACCCCCCACCTGGC
GACAGGTGCCTCTGCGGCCAAAAGCCACGTGTATAAGATACACCTGCAAAGGCGGC
ACAACCCCAGTGCCACGTTGTGAGTTGGATAGTTGTGGAAAGAGTCAAATGGCTCTC
CTCAAGCGTATTCAACAAGGGGCTGAAGGATGCCCAGAAGGTACCCCATTGTATGG
GATCTGATCTGGGGCCTCGGTGCACATGCTTTACATGTGTTTAGTCGAGGTTAAAAA
AACGTCTAGGCCCCCCGAACCACGGGGACGTGGTTTTCCTTTGAAAAACACGATGAT
AATATGGCCACAACCATGGTGAGCAAGGGCGAGGAGCTGTTCACCGGGGTGGTGCC
CATCCTGGTCGAGCTGGACGGCGACGTAAACGGCCACAAGTTCAGCGTGTCCGGCG
AGGGCGAGGGCGATGCCACCTACGGCAAGCTGACCCTGAAGTTCATCTGCACCACC
GGCAAGCTGCCCGTGCCCTGGCCCACCCTCGTGACCACCCTGACCTACGGCGTGCAG
TGCTTCAGCCGCTACCCCGACCACATGAAGCAGCACGACTTCTTCAAGTCCGCCATG
CCCGAAGGCTACGTCCAGGAGCGCACCATCTTCTTCAAGGACGACGGCAACTACAA
GACCCGCGCCGAGGTGAAGTTCGAGGGCGACACCCTGGTGAACCGCATCGAGCTGA
AGGGCATCGACTTCAAGGAGGACGGCAACATCCTGGGGCACAAGCTGGAGTACAAC
TACAACAGCCACAACGTCTATATCATGGCCGACAAGCAGAAGAACGGCATCAAGGT
GAACTTCAAGATCCGCCACAACATCGAGGACGGCAGCGTGCAGCTCGCCGACCACT
ACCAGCAGAACACCCCCATCGGCGACGGCCCCGTGCTGCTGCCCGACAACCACTAC
CTGAGCACCCAGTCCGCCCTGAGCAAAGACCCCAACGAGAAGCGCGATCACATGGT
CCTGCTGGAGTTCGTGACCGCCGCCGGGATCACTCTCGGCATGGACGAGCTGTACAA
GTAAGTCGACAATCAACCTCTGGATTACAAAATTTGTGAAAGATTGACTGGTATTCT
TAACTATGTTGCTCCTTTTACGCTATGTGGATACGCTGCTTTAATGCCTTTGTATCAT
GCTATTGCTTCCCGTATGGCTTTCATTTTCTCCTCCTTGTATAAATCCTGGTTGCTGTC
TCTTTATGAGGAGTTGTGGCCCGTTGTCAGGCAACGTGGCGTGGTGTGCACTGTGTT
TGCTGACGCAACCCCCACTGGTTGGGGCATTGCCACCACCTGTCAGCTCCTTTCCGG
GACTTTCGCTTTCCCCCTCCCTATTGCCACGGCGGAACTCATCGCCGCCTGCCTTGCC
CGCTGCTGGACAGGGGCTCGGCTGTTGGGCACTGACAATTCCGTGGTGTTGTCGGGG
AAGCTGACGTCCTTTCCATGGCTGCTCGCCTGTGTTGCCACCTGGATTCTGCGCGGG
ACGTCCTTCTGCTACGTCCCTTCGGCCCTCAATCCAGCGGACCTTCCTTCCCGCGGCC
TGCTGCCGGCTCTGCGGCCTCTTCCGCGTCTTCGCCTTCGCCCTCAGACGAGTCGGAT
CTCCCTTTGGGCCGCCTCCCCGCCTGGAATTCGAGCTCGAGCTTGTTAACATCGATA
AAATAAAAGATTTTATTTAGTCTCCAGAAAAAGGGGGGAATGAAAGACCCCACCTG
TAGGTTTGGCAAGCTAGCTTAAGTAACGCCATTTTGCAAGGCATGGAAAAATACATA
ACTGAGAATAGAGAAGTTCAGATCAAGGTCAGGAACAGATGGAACAGCTGAATATG
GGCCAAACAGGATATCTGTGGTAAGCAGTTCCTGCCCCGGCTCAGGGCCAAGAACA
GATGGAACAGCTGAATATGGGCCAAACAGGATATCTGTGGTAAGCAGTTCCTGCCC
CGGCTCAGGGCCAAGAACAGATGGTCCCCAGATGCGGTCCAGCCCTCAGCAGTTTC
TAGAGAACCATCAGATGTTTCCAGGGTGCCCCAAGGACCTGAAATGACCCTGTGCCT
TATTTGAACTAACCAATCAGTTCGCTTCTCGCTTCTGTTCGCGCGCTTCTGCTCCCCG
AGCTCAATAAAAGAGCCCACAACCCCTCACTCGGGGCGCCAGTCCTCCGATTGACT
GAGTCGCCCGGGTACCCGTGTATCCAATAAACCCTCTTGCAGTTGCATCCGACTTGT
GGTCTCGCTGTTCCTTGGGAGGGTCTCCTCTGAGTGATTGACTACCCGTCAGCGGGG
GTCTTTCATTTCCGACTTGTGGTCTCGCTGCCTTGGGAGGGTCTCCTCTGAGTGATTG
ACTACCCGTCAGCGGGGGTCTTCACATGCAGCATGTATCAAAATTAATTTGGTTTTTT
TTCTTAAGTATTTACATTAAATGGCCATAGTTGCATTAATGAATCGGCCAACGCGCG
GGGAGAGGCGGTTTGCGTATTGGCGCTCTTCCGCTTCCTCGCTCACTGACTCGCTGC
GCTCGGTCGTTCGGCTGCGGCGAGCGGTATCAGCTCACTCAAAGGCGGTAATACGGT
TATCCACAGAATCAGGGGATAACGCAGGAAAGAACATGTGAGCAAAAGGCCAGCA
AAAGGCCAGGAACCGTAAAAAGGCCGCGTTGCTGGCGTTTTTCCATAGGCTCCGCC
CCCCTGACGAGCATCACAAAAATCGACGCTCAAGTCAGAGGTGGCGAAACCCGACA
GGACTATAAAGATACCAGGCGTTTCCCCCTGGAAGCTCCCTCGTGCGCTCTCCTGTT
CCGACCCTGCCGCTTACCGGATACCTGTCCGCCTTTCTCCCTTCGGGAAGCGTGGCG
CTTTCTCATAGCTCACGCTGTAGGTATCTCAGTTCGGTGTAGGTCGTTCGCTCCAAGC
TGGGCTGTGTGCACGAACCCCCCGTTCAGCCCGACCGCTGCGCCTTATCCGGTAACT
ATCGTCTTGAGTCCAACCCGGTAAGACACGACTTATCGCCACTGGCAGCAGCCACTG
GTAACAGGATTAGCAGAGCGAGGTATGTAGGCGGTGCTACAGAGTTCTTGAAGTGG
TGGCCTAACTACGGCTACACTAGAAGGACAGTATTTGGTATCTGCGCTCTGCTGAAG
CCAGTTACCTTCGGAAAAAGAGTTGGTAGCTCTTGATCCGGCAAACAAACCACCGCT
GGTAGCGGTGGTTTTTTTGTTTGCAAGCAGCAGATTACGCGCAGAAAAAAAGGATCT
CAAGAAGATCCTTTGATCTTTTCTACGGGGTCTGACGCTCAGTGGAACGAAAACTCA
CGTTAAGGGATTTTGGTCATGAGATTATCAAAAAGGATCTTCACCTAGATCCTTTTA
AATTAAAAATGAAGTTTGCGGCCGGCCGCAAATCAATCTAAAGTATATATGAGTAA
ACTTGGTCTGACAGTTACCAATGCTTAATCAGTGAGGCACCTATCTCAGCGATCTGT
CTATTTCGTTCATCCATAGTTGCCTGACTCCCCGTCGTGTAGATAACTACGATACGGG
AGGGCTTACCATCTGGCCCCAGTGCTGCAATGATACCGCGAGACCCACGCTCACCG
GCTCCAGATTTATCAGCAATAAACCAGCCAGCCGGAAGGGCCGAGCGCAGAAGTGG
TCCTGCAACTTTATCCGCCTCCATCCAGTCTATTAATTGTTGCCGGGAAGCTAGAGTA
AGTAGTTCGCCAGTTAATAGTTTGCGCAACGTTGTTGCCATTGCTACAGGCATCGTG
GTGTCACGCTCGTCGTTTGGTATGGCTTCATTCAGCTCCGGTTCCCAACGATCAAGG
CGAGTTACATGATCCCCCATGTTGTGCAAAAAAGCGGTTAGCTCCTTCGGTCCTCCG
ATCGTTGTCAGAAGTAAGTTGGCCGCAGTGTTATCACTCATGGTTATGGCAGCACTG
CATAATTCTCTTACTGTCATGCCATCCGTAAGATGCTTTTCTGTGACTGGTGAGTACT
CAACCAAGTCATTCTGAGAATAGTGTATGCGGCGACCGAGTTGCTCTTGCCCGGCGT
CAACACGGGATAATACCGCGCCACATAGCAGAACTTTAAAAGTGCTCATCATTGGA
AAACGTTCTTCGGGGCGAAAACTCTCAAGGATCTTACCGCTGTTGAGATCCAGTTCG
ATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGAC
ACGGAAATGTTGAATACTCATACTCTTCCTTTTTCAAT

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A method of treating traumatic brain injury (TBI) comprising converting reactive astrocytes to functional neurons by providing exogenous neurogenic differentiation 1 (NeuroD1) to at least one reactive astrocyte in a damaged region of a subject's brain.

2. The method of claim 1, wherein the TBI is a closed head injury.

3. The method of claim 1, wherein the damaged region of the brain comprises non-functional neurons and reactive astrocytes due to the TBI.

4. The method of claim 3, wherein the non-functional neurons are selected from the group consisting of dead and dying neurons.

5. The method of claim 3, wherein the non-functional neurons are detected by a functional MRI (fMRI).

6. The method of claim 3, wherein the presence of non-functional neurons and reactive astrocytes in the damaged region is not primarily due to bleeding in the damaged region.

7. The method of claim 3, wherein the presence of non-functional neurons and reactive astrocytes in the damaged region is not primarily due to ischemia in the damaged region.

8. The method of claim 1, wherein providing the exogenous NeuroD1 comprises administering a recombinant expression vector to the subject, wherein the recombinant expression vector comprises a nucleic acid sequence encoding NeuroD1.

9. The method of claim 1, wherein providing the exogenous NeuroD1 comprises administering a recombinant expression vector to the subject, wherein the recombinant expression vector is a viral expression vector comprising a nucleic acid sequence encoding NeuroD1.

10. The method of claim 1, wherein providing the exogenous NeuroD1 comprises administering a recombinant expression vector to the subject, wherein the recombinant expression vector is a recombinant adeno-associated virus expression vector, and wherein the recombinant adeno-associated virus vector comprises a nucleic acid sequence encoding NeuroD1.

11. The method of claim 8, wherein the nucleic acid sequence encoding NeuroD1 is operably linked to a promoter.

12. The method of claim 11, wherein the promoter is a glial-cell specific promoter.

13. The method of claim 12, wherein the glial-cell specific promoter is a glial fibrillary acidic protein (GFAP) promoter.

14. The method of claim 13, wherein the GFAP promoter is a human GFAP (hGFP) promoter.

15. The method of claim 1, wherein no exogenous transcription factor other than NeuroD1 is provided to the at least one reactive astrocyte.

16. The method of claim 1, wherein the subject is human.

17. The method of claim 1, wherein providing the exogenous NeuroD1 comprises providing exogenous NeuroD1 to the at least one reactive astrocyte at a first treatment time in the range of about two days to about ten days after the traumatic brain injury.

18. The method of claim 1, wherein the traumatic brain injury causes a period of astrogliosis in the damaged region, and wherein providing the exogenous NeuroD1 comprises providing exogenous NeuroD1 to the at least one reactive astrocyte at a first treatment time during the period of astrogliosis or within 4 weeks after the period of astrogliosis.

19.-20. (canceled)

21. The method of claim 1, wherein the NeuroD1 comprises an amino acid sequence selected from the group consisting of: SEQ ID NO: 2, SEQ ID NO: 4, a functional fragment of SEQ ID NO: 2, a functional fragment of SEQ ID NO: 4, an amino acid sequence having at least 85% identity to SEQ ID NO: 2, and an amino acid sequence having at least 85% identity to SEQ ID NO: 4.

22. The method of claim 21, wherein the NeuroD1 is encoded by a nucleic acid sequence comprising SEQ ID NO: 1, a nucleic acid sequence having at least 85% identity to SEQ ID NO: 1, a nucleic acid sequence comprising SEQ ID NO: 3, or a nucleic acid sequence having at least 85% identity to SEQ ID NO: 3.
$$23. - 31. ( Canceled )$$
